(19) United States
(12) Patent Application Publication
(10) Pub. No.: US 2022/0273926 A1
(43) Pub. Date: Sep. 1, 2022

(54) DRUG DELIVERY USING MICRONEEDLE ARRAYS

(71) Applicant: BOARD OF REGENTS OF THE UNIVERSITY OF NEBRASKA, Lincoln, NE (US)

(72) Inventors: Ali TAMAYOL, Lincoln, NE (US); Hossein DERAKHSHANDEH, Lincoln, NE (US); Pooria MOSTAFALU, Penfield, NY (US)

(21) Appl. No.: 17/625,689

(22) PCT Filed: Jul. 8, 2020
(86) PCT No.: PCT/US2020/041241

§ 371 Date: Jan. 7, 2022

Related U.S. Application Data
(60) Provisional application No. 62/871,477, filed on Jul. 8, 2019.

(51) Int. Cl. A61M 37/00; A61M 5/142

(52) U.S. Cl. CPC A61M 37/0015; A61M 5/14248; A61M 5/14228; A61M 2037/0046; A61M 2037/0023; A61M 2037/0061; A61M 2205/3576

(57) ABSTRACT

The disclosure relates to improved wound healing compositions, methods, and systems. Embodiments of the disclosure provide microneedle arrays and devices for treating wounds and bacterial infections. Also provided are methods of using microneedle arrays and devices to passively or actively deliver various therapeutic agents to target tissues including acute and chronic wounds.

### CROSS-REFERENCE TO RELATED APPLICATIONS

This application claims priority to U.S. Provisional Application Ser. No. 62/871,477, filed Jul. 8, 2019, which is incorporated herein by reference in its entirety.

### BACKGROUND

Chronic wounds are one of the most challenging complications of diabetes and are the leading cause of non-traumatic limb amputation. Current strategies for treating chronic wounds, which include cleansing, debridement, oxygenation, antibiotics, and surgery, are expensive, time consuming, and often ineffective. A number of proteins, factors, and compounds with promising therapeutic results in vitro and in pilot animal studies have been identified for the treatment of chronic wounds, however, their clinical translation has been limited. Given the range of disruptable processes necessary for wound healing, different pharmacological agents are needed at different stages of tissue regeneration. To date, there are no wound dressings configured for precise temporal control over the independent release of various drugs plus discrete delivery of such drugs into deeper layers of the wound bed, which are typically covered by a wound crust and necrotic tissue. Accordingly, there remains an unmet need in the art for improved wound healing compositions and methods that overcome these limitations.

### BRIEF SUMMARY OF THE DISCLOSURE

In a first aspect, provided herein is a microneedle device. The microneedle device can comprise or consist essentially of an array of microneedles on a substrate, a reservoir configured to hold a therapeutic agent, and an applicator that, when placed at a wound site, administers the therapeutic agent from the reservoir to the wound site via the microneedles. Each microneedle of the array can have a needle length of about 0.8 mm to about 3 mm and a base size of about 0.5 mm to about 1.5 mm. The microneedles can be hollow and have an opening diameter of about 0.2 mm to about 0.5 mm. The microneedles can have a cone shape, a pyramid shape, a cylinder shape, a prism shape, or a pencil-like shape. The distal end of each hollow microneedle can comprise a hole. Each microneedle of the array can be a solid microneedle. The substrate can be characterized by its flexibility such that when the substrate contacts an object it substantially conforms to the object's surface. The applicator can be a micropump (or manual pump or syringe) in fluid communication with the reservoir. The device can further comprise a control module configured to communicate with the applicator and an external source. The control module can wirelessly communicate with the external source. The external source can be a low-energy Bluetooth module. The microneedles can comprise a thermoplastic resin. The microneedles can comprise a biodegradable resin. The microneedles can be produced by three-dimensional printing. The device can further comprise a potentiometric pH sensor.

In another aspect, provided herein is a method of treating a wound. The method can comprise or consist essentially of: (a) applying a wound care product to the wound, the wound care product comprising an array of microneedles on a flexible substrate, a reservoir configured to hold a therapeutic agent, and an applicator for administering the therapeutic agent from the reservoir to a wound via the microneedles; and (b) applying pressure to the wound care product such that hollow microneedles of the array penetrate the wound, thereby transdermally or intradermally administering the therapeutic agent to the wound via the penetrating microneedles and treating the wound. The wound care product can be a programmable wound dressing comprising a control module coupled to and in communication with the microneedle array and the applicator, and configured for controlling transdermal or intradermal delivery of the therapeutic agent from the reservoir to the wound via the microneedle array. At least one microneedle of the array can be hollow and have a needle length of about 0.8 mm to about 3 mm, a base size of about 0.5 mm to about 1.5 mm, and an opening diameter of about 0.2 mm to about 0.5 mm. The microneedle array can comprise one or more solid microneedles. The wound can be a chronic wound. The chronic wound can comprise a bacterial infection or biofilm. The one or more therapeutic agents can be selected from a growth factor, an antibacterial, an anti-infection agent, anti-inflammatory agent, and an anti-biofilm agent, or a combination thereof. The growth factor can be vascular endothelial growth factor (VEGF).

In another aspect, provided herein is an integrated system for wound monitoring and treatment. The system can comprise or consist essentially of (a) a flexible substrate comprising an array of microneedles configured for transdermal or intradermal delivery of at least one therapeutic agent for treating a wound; (b) a reservoir comprising a micropump, a manual pump, or a syringe, and the at least one therapeutic agent; and (c) a control module coupled to and in communication with the microneedle array and the micropump (or manual pump or syringe), and configured for controlling delivery of the at least one therapeutic agent from the reservoir to a wound via microneedles of the array. At least one microneedle of the array can be hollow and have a needle length of about 0.8 mm to about 3 mm, a base size of about 0.5 mm to about 1.5 mm, and an opening diameter of about 0.2 mm to about 0.5 mm. The system can further comprise one or more one or more testing devices for detecting at least one biological marker of a bacterial infection or inflammation. The at least one biological marker can be pH and the at least one testing device is a potentiometric pH sensor.

In a further aspect, provided herein is a method of treating a bacterial in a subject in need thereof. The method can comprise, or consist essentially of, (a) applying a wound care product to a tissue comprising or suspected of comprising a bacterial infection, the wound care product comprising an array of microneedles on a flexible substrate, a reservoir configured to hold a therapeutic agent, and an applicator for administering the therapeutic agent from the reservoir to the tissue via the microneedles; and (b) applying pressure to the wound care product such that microneedles of the array penetrate the tissue, thereby transdermally or intradermally administering the therapeutic agent to the tissue via the penetrating microneedles and treating the bacterial infection. The wound care product can be a programmable wound dressing comprising a control module coupled to and in communication with the microneedle array and the applicator, and configured for controlling transdermal or intradermal delivery of the therapeutic agent from the reservoir to the wound via the microneedle array. At least one microneedle of the array can be hollow and have a needle length of about 0.8 mm to about 3 mm, a base size of about 0.5 mm to about 1.5 mm, and an opening diameter of about 0.2 mm to about 0.5 mm. The bacterial infection can be located in an open wound. The one or more therapeutic agents can be selected from a growth factor, an antibacterial, an anti-infection agent, anti-inflammatory agent, and an anti-biofilm agent. The growth factor can be vascular endothelial growth factor (VEGF).

In another aspect, provided herein is a method for transdermal membrane delivery of a therapeutic agent. The method can comprise or consist essentially of (a) applying a microneedle device to skin, the microneedle device comprising an array of microneedles on a flexible substrate, a reservoir configured to hold a therapeutic agent, and an applicator for administering the therapeutic agent from the reservoir to the skin via the microneedles; and (b) applying pressure to the microneedle device such that microneedles of the array penetrate the skin, thereby transdermally or intradermally administering the therapeutic agent to the skin via the penetrating hollow microneedles. At least one microneedle of the array can be hollow and have a needle length of about 0.8 mm to about 3 mm, a base size of about 0.5 mm to about 1.5 mm, and an opening diameter of about 0.2 mm to about 0.5 mm. The microneedle device can be a programmable microneedle device comprising a control module coupled to and in communication with the microneedle array and the applicator, and configured for controlling transdermal or intradermal delivery of the therapeutic agent from the reservoir to the skin via the microneedle array. At least one microneedle of the array can be solid and have a needle length of about 0.8 mm to about 3 mm and a base size of about 0.5 mm to about 1.5 mm. The one or more therapeutic agents can be selected from a growth factor, an antibacterial, an anti-infection agent, anti-inflammatory agent, and an anti-biofilm agent. The growth factor can be vascular endothelial growth factor (VEGF).

These and other advantages and features of the invention will become more apparent from the following detailed description of the preferred embodiments of the invention when viewed in conjunction with the accompanying drawings.

### BRIEF DESCRIPTION OF THE DRAWINGS

FIGS. 1A-1B illustrate an exemplary embodiment of a wirelessly-controlled smart bandage equipped with MNAs for delivery of therapeutics. (A) Schematic of the engineered bandage and its operation. (B) A representative photograph of the bandages for the delivery of multiple drugs. The system comprises two modules: (1) a wearable bandage with integrated MNAs connected to (2) a controlling module, which could wirelessly communicate with a smartphone in order to control the drug delivery rate.

FIGS. 2A-2G demonstrate fabrication and characterization of 3D printed MNAs. (A) Schematic of 3D printing MNAs showing the (i) fabrication process and (ii) a representative computer model view of the designed hollow MNAs. (B) Microscopic image of sample MNAs. (C) SEM images of i) front view and ii) top view of a single hollow microneedle. (D) Multi-material printing of MNAs for creating rigid needles on flexible base. (E) The characterizing of mechanical properties of MNA islands (4 needles). (F) Microscopic view of MNA (i) before and (ii) after pig skin penetration test. (G) Characterizing insertion and pull out force of the MNA islands applied to pig skin and image of pig skin targeted with painted MNA.

FIGS. 3A-3I present characterization of microcontroller and wireless software. (A) Photograph of a typical fabricated flexible bandage with bonded hollow MNAs on PDMS-made bandage. (B) A representative stress-strain curve of peel-off test for assessment of the bonding strength resin to PDMS. (C) The bonding strength of resin and PDMS substrates (n=6). (D) Photograph of an exemplary micropump for use in a drug delivery system of this disclosure. (E) Schematic view of microcontroller and including its components. (F) Example of integrated system operation on human body. (G) Calibration plot of the micropump flow rate as a function of applied voltage. (H, I) The cumulative delivered solution using two independent pumps on a single bandage subjected different periodic functions of applied voltage considering rate of 200 μL every 6 minutes (min) for pump 1 and 400 μL every 10 min for pump 2.

FIGS. 4A-4G demonstrate characterization of drug release and its effect on cellular cultures. (A, B) The concertation of BSA and cefazolin in solutions pumps through the MNAs over time. The results suggest an insignificant change in concentration. (C) Schematic of the two-compartment in vitro model used for simulating chronic wounds covered by a crust and a necrotic tissue used for comparing the topical and MNA-based drug delivery. (D) The concentration of the BSA concertation at the bottom chamber representing the wound bed after the administration of 20 μg/mL solution of BSA through 2 mm thick agarose gel (3% w/v) within a cell culture insert (E) The drug concertation after 5, 60, and 180 min post drug administration (***P<0.001, ****P<0.0001). (F, G) Scratch assay on the culture of HUVECs receiving the following treatments: 1) 50 ng/mL of VEGF in the culture medium (positive control); 2) no VEGF (negative control); 3) equivalent to 50 ng/mL delivered topically; 4) equivalent to 50 ng/mL delivered using the MNAs. Representative micrographs are shown in G.

FIGS. 5A-5D demonstrate animal studies conducted to assess the effectiveness of VEGF delivery by MNAs on diabetic wound healing. Full thickness wounds (1 cm×1 cm) were formed on the dorsum of diabetic mice. (A) Representative images showing wound healing progression in three mice groups (control (no VEGF) (n=4), topically applied VEGF (n=4), and MNA-based VEGF delivery (n=5)) over 19 days. (B, C) Significant increase with average of 95% wound closure observed in MNA-based VEGF delivery group while no delivery and topical delivery remained with average healing rate of 40% and 50%, respectively over time (*P<0.05, **P<0.01, ***P<0.001). Wound closure rate was calculated as the ratio (percentage) of the open wound area at each measured time point divided by the area of the wound at time 0 (Day 1). (D) H&E staining for characterization of granulation and neovascularization as well as hair growth in skin in all three study groups shown in two different magnification.

FIG. 6 presents data from a Live-Dead assay of 3D printed hollow microneedles, demonstrating good cell viability.

FIG. 7 presents data from a PrestoBlue assay of 3D printed hollow microneedles, demonstrating continued cell proliferation.

FIG. 8 is a graph of erosion of hollow MNA in wound-mimic solution. The data show no change in mass of MNA over time.

FIGS. 9A-9D demonstrate mechanical strength of MNAs. (A) Image of MNA under compression by a mechanical tester machine. (B) Characterizing of required force for deformation of MNA. (C) Microscopic view of MNA before and (D) after compression test.

FIG. 10 is a schematic illustrating bandage fabrication with following steps i) Coating MNA with APTES via silanizing process inside the desiccator, ii) activating APTES by oxidation process, iii) fabrication flexible base layer for bonding to MNA, iv) oxidation, v) fabrication of microchannel system using micromolding, vi) preparing for bonding by oxidation, and vii) combining all parts and heating underweight for perfect bonding.

FIGS. 11A-11B demonstrate bonding strength tests on bonded resin to PDMS. (A) Bonded sample under tension force (B) Separated resin sample from PDMS sample after tensile test, showing required intermolecular bonding force in PDMS is less than that between resin and PDMS.

FIG. 12. Screenshot of wireless application with adjustable time interval and dose volume.

FIG. 13 is a photograph of a printed MNA comprising a drug reservoir.

FIG. 14 demonstrates that, on comparison of all treatment groups on post-surgery day 19, only MNA-perfused VEGF stimulated hair growth and promoted complete healing in the mouse model.

FIGS. 15A-15D demonstrate intradermal VEGF delivery methods for accelerated wound healing. (a) Schematic illustration of a chronic wound and drug delivery via miniaturized needle array (MNA, i), topical administration (TOP, ii), and liquid jet injector (LJI, iii). (b) Different tools employed in this study including MNA (i), TOP (ii) and LJI (iii) for administration of the drug. Insets show the micrograph of the 3D printed miniaturized hollow needles and micro-nozzle, respectively in MNA and LJI systems. (c) Representative intensity map of the Rhodamine B delivered via MNA, TOP, and LJI in an agarose gel block. (d) Quantitative evaluation of fluorescent intensity along the dashed lines in (c).

FIGS. 16A-16D demonstrate delivery kinetics of various therapeutics into a wound model. (a) Schematic illustration of in vitro model exploited for mimicking wound environment covered by eschar for characterization of release kinetics in TOP-, MNA- and LJI-based drug delivery strategies. The model is composed of two components, an insert covered with an agarose gel layer representing the eschar and granulation tissue and a bottom well mimicking the endothelial cells' environment. The release kinetics of (b) BSA as a protein, (c) Cefazolin as an antibiotic and (d) rhodamine B as a fluorescent small molecule are evaluated using different delivery strategies. The target concentration of all reagents in the wound bed model considered 20 μg/mL.

FIGS. 17A-17D demonstrate intradermal VEGF delivery for enhanced diabetic wound healing in vivo. (a) Representative images demonstrating wound healing progression in six study groups including test groups receiving VEGF (1) topically (TOP VEGF, n=6), (2) through MNAs (MNA VEGF, n=5), and (3) through LJI (LJI VEGF, n=6), as well as control groups receiving (4) PBS through MNAs (MNA PBS, n=3) (5) PBS through LJI (LJI PBS, n=3), and (6) no treatment (NGT CTRL, n=7). All groups received two treatments on day 5 and day 7 post-surgery. (b) Quantitative analysis of wound closure over time. (c) H&E staining for characterization of wound healing indicators. The underlying granulation tissue are marked with black arrowheads and hair follicles is indicated with white arrows. (d) IHC staining for evaluation of the effectiveness of VEGF delivery using different strategies. Vessels are visible in brown color, shown by white arrows. Overall, 19 days after surgery, the groups that had received VEGF intradermally demonstrated improved healing compared to topical delivery and controls. Scale bars are 1 cm, 200 μm and 100 μm, respectively in (a), (c) and (d). (*P<0.05).

FIGS. 18A-18C demonstrate advanced wound healing by intradermal VEGF delivery in large animal models. (a) Schematic illustration of contraction and re-epithelialization for wound healing in pig and human. Quantitative (b) and qualitative (c) evaluations demonstrate a significant reduction in wound contraction and therefore an increase in regenerative wound closure 14 days post-surgery. (*P<0.05, ***P<0.0005).

FIGS. 19A-19B demonstrate immunohistochemical analysis for evaluating the effectiveness of intradermal VEGF delivery in large animal wound models. Quantitative (a) and qualitative (b) expression of various markers indicating the condition of the healed tissue. Smooth muscle actin (SMA), CD3 and von Willebrand Factor (vWF) respectively indicate the presence of contractile myofibroblasts, inflammation and angiogenesis in the harvested tissue. (*P<0.05).

FIG. 20A-20C demonstrate intradermal delivery tools used in the study of Example 2. (a) Schematic demonstration of MNAs fabrication process. (b) Fabricated MNA connected to a Luer lock syringe. (c) Commercially available liquid jet injector (THRESA, China) with disposable drug injection nozzle.

FIG. 21 demonstrates application of an agarose (5% w/v) gel layer between the LJI nozzle and mouse skin for preventing tissue injury.

FIG. 22 demonstrates about 16% percent weight increase in all animals over the course of 19-days study but there is no significant difference among study groups.

FIGS. 23A-23B demonstrate lower magnification of histological analysis for evaluating the effect of intradermal VEGF delivery on the healing of chronic diabetic wounds 19 post-injury. (a) H&E staining. (b) IHC staining against CD31. Scale bars are 400 μm and 200 respectively in (a) and (b).

FIG. 24 demonstrates total macroscopic wound healing.

FIG. 25 presents normalized signals for immunohistochemical staining indicated in FIGS. 19a-19b. Data are normalized with respect to wound contraction.

FIGS. 26A-26C illustrate the use of miniaturized needles arrays for improving the bioavailability of drugs at the desired tissue depth. (a) Schematic showing delivery of keratinocyte growth factor (KGF) in blue to the epidermis of the skin, and vascular endothelial growth factor (VEGF) in red past the epidermis to improve neovascularization. (b) Fabricated multilevel needle array where each needle length is filled with different dyed gel, and (c) distribution of dye in agarose from different needle lengths.

FIGS. 27A-27F illustrate MNA fabrication and a gel-loading process. (a) 3D printing of microneedles and support material removal with NaOH. (b) Solidworks rendering of the semi-flexible microneedle array. (c) Demonstration of the flexibility of fabricated semi-flexible microneedle array. (d) Concept drawing for 36 needle array gel-loading pipette attachment. (e) Demonstration of rhodamine B dyed alginate deposition using hydrogel loading attachment. (f) Fluorescent and SEM imaging of gel-filled microneedle.

FIGS. 28A-28G demonstrate mechanical properties and penetration testing. (a) Microneedle array in the mechanical tester for compression test. (b) Force-displacement curve for needle compression test. (c) Needle bending resulting from compression. (d) Pig skin insertion testing configuration. (e) Force-displacement curves for pig skin insertion tests, for microneedle arrays with completely flexible backing, non-flexible backing, and semi-flexible backing (f) Pig skin after insertion of multilevel microneedle array with 3 mm needles containing red fluorescent dye and 2 mm needles containing green fluorescent dye. (g) Alginate gel loaded in microneedle before and after insertion into pig skin.

FIGS. 29A-29C demonstrate SEM imaging and release studies. (a) Filled microneedle resin-alginate interface at (i) ×103 magnification, (ii) ×239 magnification, and (iii) ×525 magnification. (b) 1% alginate pore structure. (c) Release profiles of MNA arrays with (i) rhodamine-B with 3 mm needles, (ii) rhodamine-B with 2 mm needles, (iii) BSA with 3 mm needles, and (iv) PEGDA with 3 mm needles.

FIG. 30 is a schematic illustration conceptual view of an automated smart bandage. The bandage can comprised of an array of flexible pH sensors and a flexible heater to trigger thermo-responsive drug carriers containing antibiotics. Thermo-responsive drug carriers are embedded in a layer of alginate hydrogel which was cast around the pH sensors and on top of the flexible heater. The sensors and heater are connected to an electronic module that could record the sensors signal and power the heater if needed. The electronic module could also communicate wirelessly to computers and smartphones.

FIGS. 31A-31K demonstrate sensors fabrication and characterization. A-C) Optical images and design of the fabricated pH sensor on PET film, carbon/PANI, and silver/silver chloride served as working electrode and reference electrode. D) Calibration plot of the pH sensor in range 4-10 with r2=0.95 and sensitivity of −50 mV pH-1. E) Transient response of the pH sensor over 24 h showing its long-term stability. F) Optical image of the flexible heater fabricated using gold electrodes on Parylene substrate. G) Calibration plot of the heater with a slope of 8° C. V-1. H) Transient response of the heater in a wet condition shows the response time of 5 min with applied 3 V. I) The temperature variation in response to cyclic application of voltage to the heater to change the platform temperature. J,K) Simulation for heat distribution inside the gel before and after applied electrical power, with consideration of the human skin showing that the generated heat influenced the hydrogel and did not impact on skin temperature.

FIGS. 32A-32H present data from a drug release study. A-C) Optical image of the particles in a range of temperatures. D-F) Fluorescence images of drug carriers embedded inside the hydrogel attached to microheater, rhodamine B was used for better visualization. G) Release profile of cefazolin antibiotics in different temperatures and conditions. H) Release rate control of cefazolin by adjusting the temperature.

FIGS. 33A-33F demonstrate a scratch test assay test for evaluation of cell migration: A) control sample made of cefazolin solution, B) alginate sample without antibiotic, and C) antibiotic patch. D) Quantitative analysis of the cell migration showing the gap size on the scratch wound assay. E,F) Viability of keratinocyte and total DNA content as an indication of cellular proliferation in presence of the antibacterial and control samples.

FIGS. 34A-34H demonstrate in vitro evaluation of the antibacterial activity of the bandage. A,B) Diffusion test for antibacterial releasing hydrogel and negative control (hydrogel without antibiotics). C) Live-dead from biofilm formation on control patch. D) Live-dead staining from biofilm formation on antibacterial patch; live bacteria appear as green. E) CFU counting test for S. aureus using cefazolin. F) Schematics of in vitro model for culturing of S. aureus bacteria in a bioreactor monitored with pH sensor and treated with the patch loaded with antibiotic. G) In vitro test showing the pH variation over time followed by the activation of the heater at pH=6.5. H) The integration of the electronic component, pH sensors, microheater, and drug-loaded hydrogel. The patch was placed on the author's hand.

### DETAILED DESCRIPTION

Although the following description refers to certain aspects or embodiments, such aspects or embodiments are illustrative and non-exhaustive in nature. Having reviewed the present disclosure, persons of ordinary skill in the art will readily recognize and appreciate that numerous other possible variations or alternative configurations or aspects are possible and were contemplated within the scope of the present disclosure.

The compositions, methods, and systems provided herein are based at least in part on the inventors' development of a wirelessly-controlled smart bandage having an integrated array of hollow and/or solid microneedles. As described herein, the inventors' microneedle arrays and wound care products comprising them are particularly well-suited for the treatment of chronic wounds.

Advantages of the compositions, methods, and systems provided herein are multifold. For example, by transdermally or intradermally delivering therapeutic agents below a wound crust and necrotic tissue, the compositions, methods, and systems provided herein are particularly effective for treating exudating wounds and, in particular, chronic wounds such as diabetic ulcers including diabetic foot ulcers. Localized, minimally invasive delivery via hollow or solid microneedles provides an active therapeutic agent directly to live tissue of wound bed, avoids dilution of the agent by the presence of exudate, and is not dependent on blood vessel delivery of systemically administered agents. Microneedle arrays are minimally invasive, thereby induce minimal pain and inflammation compared to other more invasive methods. In addition, microneedle arrays can be equipped with a programmable electric driver for controlling the active drug delivery using micropumps, manual pumps, syringes, and the like. In addition, such microneedle array systems can be configured to communicate wirelessly with smartphone and control the time point and dosage of different therapeutics simultaneously via specifically designed applications.

Compositions of the Disclosure

Accordingly, in a first aspect, provided herein are compositions comprising hollow and/or solid microneedles. As used herein, the term “microneedle” refers to short needles that are sufficiently small in size to pass the stratum corneum but not hit the nerves underneath. As described herein, microneedles preferably have a needle length of about 0.8 mm to about 3 mm, have a base size of about 0.5 mm to about 1.5 mm, and (for hollow microneedles) have an opening diameter of about 0.2 mm to about 0.5 mm. Hollow microneedles have a hollow core that, when penetrating a tissue of interest, facilitates delivery of drugs into the region of interest. Solid microneedles may have a partially or fully solid core and facilitate delivery of drugs to a region of interest by penetrating the tissue of interest.

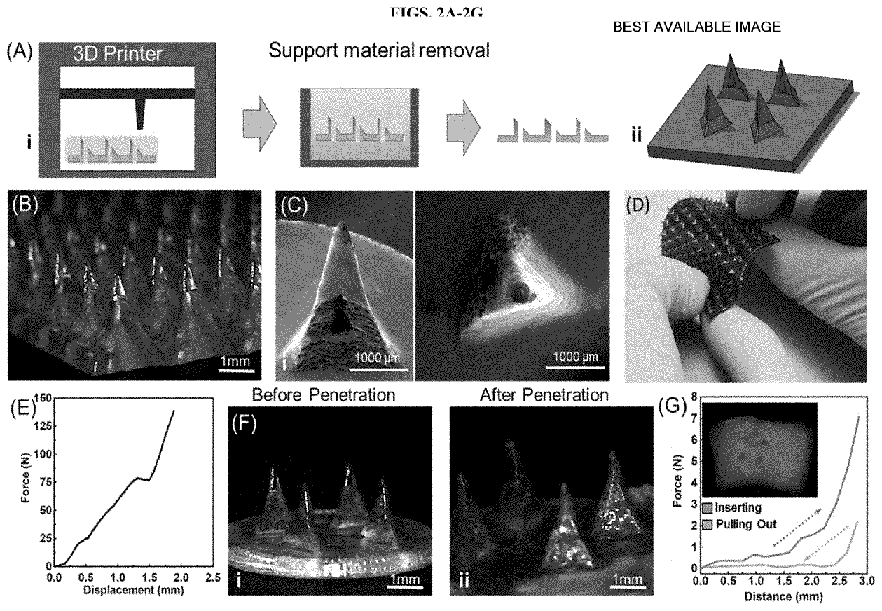

In some cases, the composition is a microneedle device comprising or consisting essentially of one or more microneedles on a flexible substrate, a reservoir configured to hold a therapeutic agent, and an applicator for administering the therapeutic agent via the microneedles. Referring to FIGS. 2A-2B, the microneedles preferably extend outwardly from the flexible substrate. Each microneedle may be in any shape as long as it can pierce the skin and/or a wound crust and penetrate necrotic tissue to reach the wound bed. Each microneedle can be various shapes such as a cone, pyramid, cylinder, prism, or pencil-like shape (meaning a shape having a column body and a cone-shaped end portion). Preferably, the microneedle has a conical or pyramidal shape. As illustrated in FIGS. 2A-2B, in some cases hollow microneedles can have an overall conical or pyramidal shape with one side comprising an opening or aperture through which a therapeutic agent (preferably, in a liquid form) can be dispensed.

In some cases, a single microneedle is provided on the support base. In other cases, a plurality of microneedles (e.g., a plurality of hollow microneedles, a plurality of solid microneedles, a plurality comprising hollow and solid microneedles) may be disposed closely together on the support base. When a plurality of microneedles are disposed, the microneedles are preferably arranged in an array (e.g., a microneedle array or “MNA”). As used herein, the term “array” means that microneedles are arranged in a specific pattern, for example in a matrix arrangement, concentric circle arrangement, or random arrangement.

As described herein, microneedles can be hollow or solid. Microneedles can be produced by any method that yields microneedles and microneedle arrays that are smooth and sharp enough to penetrate wounded skin. For example, microneedles and arrays thereof can be produced by three-dimensional (3D) printing using a suitable resin. The resin can be a thermoplastic resin. In some cases, the resin is biodegradable.

In some cases, the microneedles are resorbable. In some cases, the microneedles are stimuli-responsive.

In some cases, microneedles can be solid, wholly or in part. When solid microneedles are used, therapeutic agents can be coated or otherwise applied to an exterior surface of the microneedle. Alternatively, therapeutic agents can be incorporated into the microneedle material. In any such cases, therapeutic agents can be delivered by solid microneedles passively (e.g., through diffusion) or actively (e.g., by heating the needles), or by incorporation of materials that are responsive to environmental conditions or environmental stimuli such as pH, temperature, level of enzymes, presence of pathogens, etc.

In preferred embodiments, a microneedle array of this disclosure is combined with any clinically acceptable superficial wound treatment article (e.g., negative pressure therapy devices, bandages, films, adhesives, and the like) to form a therapeutic composition (e.g., therapeutic device). For example, in some cases, a microneedle array of this disclosure is attached to or integrated into a flexible wound treatment article such as a bandage base to form a microneedle device. In such cases, the microneedle device comprises a flexible substrate and plurality of microneedles extending outwardly therefrom. As described herein, the microneedles are configured to bypass the wound crust and the necrotic tissue. Upon placement of the flexible substrate onto skin or a wound therein, the microneedles pierce the skin and any wound crust on the skin and preferably penetrate the skin deep enough to reach live tissue of a wound bed.

Preferably, the therapeutic composition is characterized by its flexibility such that when the article contacts an object it substantially conforms to the object's surface. For instance, the therapeutic composition can comprise a substrate that is a flexible bandage such that, upon contacting to a portion of a patient's skin, the composition substantially conforms to the patient's skin. Preferably, the bandage base is made of a thin, flexible material. In some cases, the flexible bandage base is made of thin films, fabrics, breathable fabrics, perforated films and fabrics, and other suitable materials including, without limitation, polymer-based films (e.g., polyethylene film, polyurethane film). In some cases, the bandage base comprises an antimicrobial material.

In some cases, the bandage base has a first side that comes into contact with a wound or tissue adjacent to or surrounding a wound. In such cases, the first side comprises an adhesive agent such that, upon placement of the first side of the bandage to a wound or wound-adjacent tissue, an adhesive connection forms between the bandage and the wound or wound-adjacent tissue. In preferred embodiments, the adhesive agent to reduces or prevents shifting of the device when contacted to a patient. In other cases, the flexible bandage base does not comprise an adhesive. Instead, upon placement of the flexible bandage at a wound site, the bandage is held in place using a self-adhesive film bandage such as Hydrofilm® or another wound dressing or wrap.

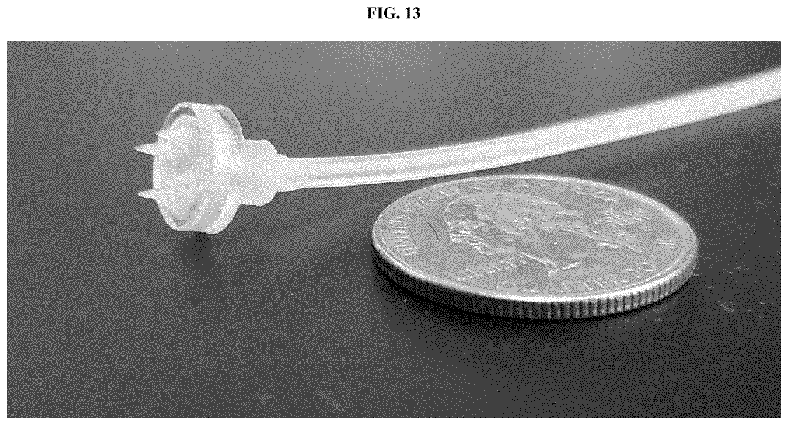

In some cases, the composition comprising hollow and/or solid microneedles further comprises one or more therapeutic agent reservoirs (see FIG. 13) and a mechanism to deploy a therapeutic agent from its reservoir into the hollow microneedles or onto solid microneedles. For instance, when hollow microneedles pierce a tissue, a therapeutic agent is deployed from its reservoir, through the hollow microneedles, and into the tissue. The mechanism can be a pump such as, for example, a peristaltic pump. Preferably, the pump is a micropump. As used herein, the term “micropump” refers to any pumping mechanism such as a peristaltic pump, manual pump, or syringe that is capable of manipulating minute amounts of a therapeutic agent (e.g., a medicament such a liquid antibiotic, a lotion comprising a therapeutic agent) for delivery in minute dosages and, preferably, small enough to be included in a wearable device. Active pumping remains one of the most accurate methods for precisely controlling the delivery rate of drug solutions.

In some cases, a composition comprising microneedles of this disclosure is a programmable bandage comprising a flexible bandage base and an integrated array of microneedles that are configured to bypass the wound crust and the necrotic tissue, thereby delivering therapeutic agents to deeper layers of a wound bed. In addition to microneedles extending outwardly from a flexible bandage base, the programmable smart bandage device comprises one or more therapeutic agent reservoirs, one or more micropumps (or manual pumps or syringes), and a control module (e.g., processor, microcontroller). In some cases, the microneedle array of a programmable bandage is connected to a control module (e.g., a processor) which may be configured to execute code or instructions to perform the operations and functionality described herein, and to perform calculations and generate commands. The control module can be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The control module may be a single processing unit, multiple processing units, a single processing core, multiple processing cores, special purpose processing cores, co-processors, or any combination thereof. According to certain example embodiments, the control module, along with other components of a computing machine, may be a virtualized computing machine executing within one or more other computing machines.

In some cases, the control module is configured to wirelessly communicate with a computer in order to control the drug delivery rate. The computer can be a smartphone having an application program to control drug delivery rates. In such cases, the microneedle device is a programmable “smart” bandage. Advantageously, wireless communication from the computer to the microneedle device permits remote control over the timing and rate of therapeutic agent delivery. In some cases, the programmable bandage is configured for delivery of two or more therapeutic agents to a tissue via the microneedles, where a computer program is used for temporal control over delivery of one or more of the therapeutic agents according to a predetermined schedule.

In some cases, a composition comprising microneedles of this disclosure comprises features described in the Examples section and in the following sections. For example, the wound treatment article will comprise in some cases a networked closed-loop automated patch for monitoring and treatment of the chronic wounds. See, e.g., Mostafalu et al., Small J. 2018, 14:1703509, which is incorporated herein by reference in its entirety. In some cases, the closed-loop patch includes one or more of an array of flexible pH sensors, a temperature sensor, a hydrogel release patch comprising thermo-responsive drug-loaded carriers, a flexible heater to trigger thermo-responsive drug carriers, and an electronics patch. In some cases, the closed-loop patch is designed and fabricated on a flexible substrate such as a PET substrate. In some cases, thermo-responsive drug carriers are embedded in a layer of flexible material (e.g., alginate hydrogel) which can be cast around the array of pH sensors and/or on top of the flexible heater. The sensors and heater can be connected to an electronic module configured to record the sensors signal and power the heater if needed. The electronic module could also communicate wirelessly to computers and smartphones.

In some cases, the microneedle device comprises a communication network. The communication network can be any suitable communication network or combination of communication networks. In some cases, the communication network includes, for example, a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, the communication network can be a local area network, a wide area network, a public network (e.g., the Internet), a private or semi-private network (e.g., a corporate, hospital, diagnostic laboratory, or university intranet), any other suitable type of network, or any suitable combination of networks.

In some cases, the microneedle device further comprises a communication system. The communication system can include any suitable hardware, firmware, and/or software for communicating information over a communication network and/or any other suitable communication networks. For example, the communications system can include one or more transceivers, one or more communication chips and/or chip sets, etc. In a more particular example, the communication system can include hardware, firmware and/or software that can be used to establish a Wi-Fi connection, a Bluetooth connection, a cellular connection, an Ethernet connection, etc.

Methods of the Disclosure

In another aspect, provided herein is a method for treating a wound in a subject. As used herein, the term “wound” includes acute wounds as well as chronic wounds in any tissue. As used herein, the term “chronic wound” refers to a wound that does not fully self-heal after 3 months and often persists for over a year. In chronic wounds, the highly-orchestrated cascade of physiological processes leading to wound healing is typically interrupted, leading to extreme hypoxia from the lack of angiogenesis, immune-modulated hyper inflammation, biofilm formation, and bacterial infection. Although individual wound pathophysiology differs, the cause of healing impairment is typically multifactorial, though most complications result from the lack of vascularization. Impaired angiogenesis significantly limits the availability of vital nutrients, oxygen, immune cells, and epithelial cells at the injury site. Without oxygen and nutrients, areas of necrotic tissue grow, which are conducive to the growth of biofilms. The formed biofilms are extremely resistant to convention treatments comprising topical and/or systemic delivery of antibiotics. Wounds for which the devices and methods of this disclosure may be applied include acute wounds (e.g., an acute traumatic laceration, including lacerations resulting from a surgical procedure) and recalcitrant or chronic wounds (e.g., a venous ulcer, a pressure sore, a decubitis ulcer, a burn, a diabetic ulcer or a chronic ulcer of unknown etiology). As used herein, the term “chronic wound” refers to those wounds which do not heal within about three months using standard treatment. The methods provided herein are particularly advantageous for treating chronic wounds, diabetic ulcers, and wounds comprising a bacterial infection and/or biofilm. Chronic wounds can be divided into three main categories: venous leg ulcers (VLUs), diabetic foot ulcers (DFUs), and pressure ulcers (PUs). VLUs are open lesions that occur between the ankle joint and the knee in patients with venous disease. These ulcers occur in advanced forms of chronic venous disorders such as varicose veins and lipodermatosclerosis. DFUs are nonhealing full-thickness wounds that extend through the dermis, below the ankle, and are often caused by repetitive injury to the site. PUs are injuries to the skin and underlying tissue due to prolonged pressure on the skin. PUs commonly occur on the skin over bony areas of the body, such as the hips, heels, tailbone, and ankles.

In some cases, the method for treating a wound in a subject comprises (a) applying a wound care product to the wound, the wound care product comprising an array of hollow and/or solid microneedles on a flexible substrate, a reservoir configured to hold a therapeutic agent, and an applicator for administering the therapeutic agent from the reservoir to a wound via the microneedles; and (b) applying pressure to the wound care product such that microneedles of the array penetrate the wound, thereby transdermally or intradermally administering the therapeutic agent to the wound via the penetrating microneedles. As a result of practicing the method, the wound is treated.

In some cases, the steps of the method are repeated, i.e., steps (a) and (b) are performed, followed by performing steps (a) and (b) again, and so on. Such a repetitive cycle may be performed any number of times, and advantageously is performed at least until any infection in the wound has been eradicated. The cycle may also be repeated until the wound has healed entirely, thereby hindering reinfection.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For purposes of this disclosure, “treating” or “treatment” describes the management and care of a patient for the purpose of combating the disease, condition, or disorder. The terms embrace both preventative, i.e., prophylactic, and palliative treatment. “Treating” includes the administration of a compound of present invention to prevent the onset of the symptoms or complications, alleviating the symptoms or complications, or eliminating the disease, condition, or disorder. The term “treat” and words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of this disclosure can provide any amount of any level of treatment or prevention of disease in a mammal. Furthermore, the treatment or prevention provided by the inventive method can include treatment or prevention of one or more conditions or symptoms of the disease or disease state, e.g., chronic wound, being treated or prevented. Also, for purposes herein, “prevention” can encompass delaying the onset of the disease, or a symptom or condition thereof.

The wound care product can be a microneedle array or device as described in this disclosure. In some cases, the wound care product is a programmable wound dressing comprising a control module coupled to and in communication with the microneedle array and the applicator, and configured for controlling transdermal or intradermal delivery of the therapeutic agent from the reservoir to the wound via the microneedle array. As described herein, microneedles of the array or device preferably have a needle length of about 0.8 mm to about 3 mm, have a base size of about 0.5 mm to about 1.5 mm, and have an opening diameter of about 0.2 mm to about 0.5 mm.

Every wound undergoes a similar reparative process, regardless of the type or degree of tissue damage. The reparative process includes three distinct phases: the inflammatory (also known as exudative) phase, the proliferative phrase, and the differentiation or regeneration phase. The inflammatory or exudative phase involves the detachment of deteriorated tissue and wound cleaning by the production of exudate. The proliferative phase includes the development of granulation tissue, the formation of which involves leukocytes, plasma cells, mast cells, and in particular fibroblasts, that promote tissue growth through the production of collagen. The differentiation or regeneration phase involves maturation of healing tissue and scar formation. Where the wound involves damage to the skin, the final stage in wound healing is epithelialization, in which epidermal cells migrate to resurface the denuded area.

For the methods of this disclosure, a hollow microneedle device or array as provided herein is applied to a wound to provide localized delivery of a therapeutic agent to the wound. The term “therapeutic agent” as used herein means an agent that provides treatment and/or prophylaxis for a particular disease state. A therapeutic effect is obtained by suppression, remission, or eradication of a disease state. Preferably, the therapeutic agent is a wound-healing agent, meaning an agent that is commonly used in wound healing and/or having properties advantageous for any of the three phases of wound healing. In some cases, the therapeutic agent is an antimicrobial agent such as an antibiotic, antiviral, antifungal, or anti-parasitic agent. Exemplary antibiotics, anti-bacterials, and anti-infectives include sulfonamides (e.g., sulfanilamide, sulfadiazine, sulfamethoxazole, sulfisoxazole, para-aminobenzoic acid, or sulfacetamide), trimethoprim-sulfamethoxazole, quinolones (e.g., ciprofloxacin, ofloxacin, or nalidixic acid), beta-lactam antibiotics such as penicillins or cephalosporins, aminoglycosides (e.g., kanamycin, tobromycin, gentamycin C, amikacin, neomycin, netilmicin, streptomycin, or vancomycin), tetracyclines, chloramphenicol, and macrolides (e.g., erythromycin, clarithromycin, or azithromycin).

In some cases, the therapeutic agent is a growth factor such as vascular endothelial growth factor (VEGF). As demonstrated in Example 2, intradermal delivery of VEFG by hollow microneedles was superior to topical VEGF administration (and controls) in promoting wound healing and vascularization in freshly formed skin in vivo assays. Furthermore, intradermal delivery of VEGF by hollow microneedles in a swine model of full thickness skin injury reduced wound contraction while it increased wound healing by regeneration. Immunohistochemical analysis further demonstrated that it enhanced the vascularization of the wound bed and reduced the level of inflammation. These findings demonstrate the importance of the delivery point and the administration of biological factors in the therapy outcome. In some cases, the therapeutic agent comprises a combination of VEGF and one or more other agents including, without limitation, an antibiotic, an anti-bacterial, an anti-infectives, a debriding agent, an immunostimulatory compound (e.g., glucocorticosteroids, non-steroidal anti-inflammatory drugs (NSAIDS), PDGF, EGF, IGF, TNF-α antagonists), and anti-IL-6. Other growth factors include, without limitation, CTGF, BMP-7, FGF-2, TGFβ, TGF-α, IL-6, anti-IL-6, IL-8/CXC chemokines, insulin like growth factor (IGF), epidermal growth factor (EGF), Cystatin, α-2 microglobulin, Pleiotrophic factor (3, NGF, NT, transthyretin, retinoic acid, PTHLH, haptocorrin, tropomodulin 3, PEDF, β-2-microglobulin, somatostatin, fibronectin, laminin β1 and secreted extracellular matrix factors.

Other suitable therapeutic agents include, without limitation, other growth factors (e.g., platelet derived growth factor (PDGF), CTGF, Bone Morphogenetic Proteins (e.g., BMP-7), fibroblast growth factors (e.g., FGF-2), TGFβ, TGF-α, IL-6, IL-8/CXC chemokines, insulin-like growth factor (IGF), epidermal growth factor (EGF), Cystatin, α-2 microglobulin, Pleiotrophic factor β, NGF, NT, transthyretin, retinoic acid, PTHLH, haptocorrin, tropomodulin 3, PEDF, β-2-microglobulin, somatostatin, fibronectin, laminin β1 and secreted extracellular matrix factors), debriding agents, hydrogels, compression bandages, foam dressings, hydrocolloids, alginate dressings, and immunostimulatory compounds (e.g., glucocorticosteroids, non-steroidal anti-inflammatory drugs (NSAIDS), PDGF, EGF, IGF, TNF-α antagonists).

In some cases, the therapeutic agent to be delivered by an array of microneedles of this disclosure is encapsulated in a substance which can release drugs actively or passively. In active delivery, the release occurs in response to environment incentives including pH, enzymes, temperature, chemical reactions and so on; or release can be triggered by external stimuli such as light, magnetic field, ultrasound, etc. In some cases, passive delivery is based on the diffusion of the drug from a carrier matrix to nearby medium. Different encapsulating substances, and combinations thereof, can be used to develop different controlled-release profiles of therapeutic agents delivered by hollow microneedles as described herein. In some cases, naturally-derived or synthetic materials are used to encapsulate a therapeutic agent for controlled release. Therapeutic agents can be encapsulated as micro-sized particle, nano-sized particles, gels, or fibers, or a combination thereof.

By way of non-limiting example, a therapeutic agent can be encapsulated in a thermo-responsive polymer such as a Poly(N-isopropylacrylamide) (pNIPAM)-based polymer for use in a microneedle array of this disclosure. PNIPAM)-based polymers have been widely used for engineering thermo-responsive drug delivery systems. The critical temperature of pNIPAM is around 32° C., which is close to skin temperature. pNIPAM is hydrophilic below its critical temperature and becomes hydrophobic above that, where the aqueous solution containing hydrophilic drug will be pushed out of the drug carriers. Due to the low critical temperature of pristine pNIPAM, it can be used for engineering systems that can release their content delivered to the wound. In other cases, a therapeutic agent can be encapsulated in an ionizable polymer for use in a microneedle array of this disclosure. Ionizable polymers are weak acids or bases used as pH-responsive materials and function through a change in their ionization state, resulting in changes in the polymer conformational state.

In some cases, the wound to be treated is a large open wound such as a chronic wounds and ulcers (e.g., diabetic ulcers). In such cases, suitable therapeutic agents comprise VEGF, PDGF, and/or other growth factors such as IGF, TGF-Beta, and FGFs.

As used herein, the term “subject” is intended to include living organisms in which the organism has a wound to be treated (e.g., humans, non-human mammals, other animals). A “subject” or “patient,” as used therein, may be a human or non-human mammal. Non-human mammals include, for example, livestock and pets, such as ovine, bovine, porcine, canine, feline and murine mammals. Preferably, the subject is human. The phrase “subject in need thereof,” is intended to refer to an animal or human subject who has been diagnosed with, is suspected of having, or is at risk of having a disease or condition (e.g., an acute wound, a chronic wound) requiring treatment with a device provided herein.

In some embodiments, the wound to be treated is exuding, which means the wound is producing a liquid exudate comprising proteins, enzymes, inflammatory cytokines, and even pathogens in response to injury. The presence of exudate, sometimes referred to as “drainage,” can wash out topically delivered therapeutics and/or deactivate them due to the presence of various enzymes and proteins. Consequently, conventional treatments are often ineffective for such wounds. Advantageously, hollow microneedle devices and hollow microneedle arrays penetrate the skin and provide for localized delivery of therapeutic agents into exuding wounds, into live tissue located beneath a wound crust and/or necrotic tissue, and into avascular wounds.

The therapeutic agent for use in connection with a microneedle device of this disclosure may optionally be formulated with one or more appropriate pharmaceutically acceptable carrier. Pharmaceutically acceptable carriers include any and all solvents, diluents, or other liquid vehicles, dispersion or suspension aids, surface active agents, isotonic agents, thickening or emulsifying agents, preservatives, solid binders, and lubricants. Remington's Pharmaceutical Sciences [Ed. by Gennaro, Mack Publishing, Easton, Pa., 1995] describes a variety of different carriers that are used in formulating pharmaceutical compositions and known techniques for the preparation thereof. In some cases, the microneedle device is used to deliver one or more additional therapeutic agents. Additional therapeutic agents may include, without limitation, growth factors, anti-inflammatory agents, topical steroids, fibronectin, collagen, B vitamins, and hyaluronic acid.

The methods of this disclosure may include administering the therapeutic agent using any amount effective for treating the subject. The exact dosage is chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; and tolerance/response to therapy. For any active agent, the therapeutically effective dose can be estimated initially either in cell culture assays or in animal models, usually mice, rabbits, dogs, or pigs. The animal model is also used to determine a desirable concentration range and route of administration.

In another aspect, provided herein is a method for treating a bacterial infection, including a bacterial infection associated with an open wound (e.g., a chronic wound, diabetic ulcer). As used herein, the term “bacterial infection” refers to any undesired presence and/or growth of bacteria in a subject. Such undesired presence of bacteria may have a negative effect on the host subject's health and well-being. While the term “bacterial infection” should not be taken as encompassing the growth and/or presence of bacteria which are normally present in the subject, for example in the digestive tract of the subject, it may encompass the pathological overgrowth of such bacteria. The term “bacterial infection” encompasses infections that bacterial infections involve several species of bacterial pathogens as well as those that involve a single bacterial species. Infections involves multiple species of bacterial pathogens are also known as complex, complicated, mixed, dual, secondary, synergistic, concurrent, polymicrobial, or co-infections.

In some cases, the method for treating a bacterial infection in a subject comprises (a) applying a wound care product to tissue comprising a bacterial infection, the wound care product comprising an array of microneedles on a flexible substrate, a reservoir configured to hold a therapeutic agent, and an applicator for administering the therapeutic agent from the reservoir to the tissue via the microneedles; and (b) applying pressure to the wound care product such that hollow microneedles of the array penetrate the tissue, thereby transdermally or intradermally administering the therapeutic agent to the tissue via the penetrating microneedles. As a result of practicing the method, the bacterial infection is treated.

Bacterial infections that can be treated according to the methods of this disclosure include, without limitation, infections produced by Streptococcus pneumoniae, Neisseria Meningitides. Haemophilus influenzae, Staphylococcus aureus, Pseudomonas aeruginosa, Streptococcus agalactiae, Listeria monocytogenes, and Escherichia coli.

Systems of the Disclosure

In another aspect, provided herein is a wound healing system that provides integrated wound monitoring and treatment. Preferably, the system is configured as a wound dressing configured for integrated wound monitoring and administering therapeutic agents via a microneedle array and according to a pre-determined therapeutic protocol.

In some cases, the integrated wound healing system comprises features described in the Examples section.

In some cases, the integrated wound healing system for wound monitoring and treatment comprises (a) a flexible substrate comprising an array of microneedles configured for transdermal delivery of at least one therapeutic agent for treating a wound; (b) a reservoir comprising a micropump, a manual pump, or a syringe, and the at least one therapeutic agent; and (c) a control module coupled to and in communication with the microneedle array and the micropump, manual pump, or syringe, and configured for controlling delivery of the at least one therapeutic agent from the reservoir to a wound via the microneedle array. In some cases, the system further comprises one or more one or more testing devices for detecting at least one biological marker of a bacterial infection or inflammation. For example, the system can include a potentiometric pH sensor to assess pH of the wound tissue prior to and/or during treatment. In other cases, the system comprises a testing device to detect and measure a level of expression of gene or gene product associated with inflammation, for which an elevated level relative to wild-type is indicative of active infection. Such genes and gene products include, without limitation, proinflammatory cytokines (e.g., TNF-α), interleukins (e.g., IL-1β, IL-4, IL-6, IL-8, IL-10, IL-18, MCP-1, MCP-2, MCP-3 (monocyte chemoattractant proteins), MIP-1α, MIP-Iβ, MIP-2 (macrophage inflammatory proteins), Interferons IFN-alpha, IFN-beta, and IFN-gamma, GM-CSF (granulocyte/macrophage colony stimulating factor), PF-4 (Platelet factor 4), and RANTES (a member of the chemokine family). Additionally, or alternatively, the system comprises a testing device to detect and measure a level of expression of various cytokines, for which a reduced level relative to wild-type is indicative of active infection. Standard diagnostic technology (e.g., immunodetection) can be used to detect and measure cytokines. Antibodies which detect cytokines may also be employed, and are available commercially. In some cases, the system provides a platform configured for in situ detection of a bacterial infection by continuously monitoring wound pH and administering therapeutic agents (e.g., antibiotics, growth factors) locally and on demand. Advantageously, the system comprises a control module (e.g., processor) configured to collect and process data measured by the sensors and to program the drug release protocol for individualized treatment.

The above methods and systems may be used in various wound healing applications, such as, but not limited to, burns, abrasions, surgical wounds, and skin grafts. Additionally, while the above methods and systems have been described with respect to wound healing, the principles described herein may applied to other tissue types for which it is advantageous to provide targeted, localized delivery of a therapeutic agent to a deep tissue layer or in the presence of fluids or a crusted surface, for which topical application of the therapeutic agent is inadequate to elicit a therapeutic effect.

Articles of Manufacture

In another aspect, provided herein is a kit comprising one or more components useful for treating a wound or infection in a subject in need thereof. Components of the kit can include one or more microneedle arrays or a device or wound dressing comprising microneedle arrays as described herein. The kit can also contain one or more therapeutic agents. In some cases, the kit also comprises instructions for treating a chronic wound in a subject in need thereof according to the methods provided herein.

In another aspect, provided herein is a kit comprising one or more components useful to treating a chronic wound in a subject in need thereof according to the methods provided herein. Components of the kit can include a computer program that communicates with a microcontroller of the microneedle device to exert temporal control over delivery of one or more therapeutic agents via a microneedle array. In some cases, the kit also comprises instructions for treating a chronic wound in a subject in need thereof according to the methods provided herein.

It should be noted that embodiments and features described in the context of one of the aspects of the present invention also apply to the other aspects of the invention.

All patent and non-patent references cited in the present application, are hereby incorporated by reference in their entirety.

This disclosure is presented to enable a person skilled in the art to make and use embodiments described herein. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

It is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting.

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Values expressed in a range format should be interpreted in a manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt % to about 5 wt %, but also the individual concentrations (e.g., 1%, 2%, 3%, and 4%) and the sub-ranges (e.g., 0.1% to 0.5%, 1.1% to 2.2%, and 3.3% to 4.4%) within the indicated range.

The invention will now be described in further details in the following non-limiting examples.

### EXAMPLES

### Example 1—Wirelessly-Controlled Smart Bandage with Integrated Microneedle Arrays for the Treatment of Chronic Wounds

Chronic wounds are one of the most challenging complications of diabetes and are the leading cause of non-traumatic limb amputation. Despite the progresses in identifying factors and promising in vitro results for treatment of chronic wounds, their clinical translation has been limited. Given the range of disruptive processes necessary for wound healing, different pharmacological agents are needed at different stages of tissue regeneration. This Example describes development of a programmable platform which is capable of actively delivering a variety of drugs with independent temporal profiles through microneedle arrays (MNAs) into deeper layers of the wound bed. In particular, this Example describes an individualized wearable, programmable bandage for treating chronic diabetic wounds. This bandage is equipped with polymeric MNAs for delivering vital pharmacological agents and growth factors. The MNAs are minimally invasive, thereby induce minimal pain and inflammation as compared to other invasive methods. Our programmable bandage allows physicians to remotely administer theraputics as needed. The data for delivery of factors such as vascular endothelial growth factor (VEGF) through the MNAs demonstrate that, in addition to the selection of suitable therapeutics, the mode of delivery and spatial distribution of microneedles within the wound bed are equally important. Administration of VEGF to chronic dermal wounds of diabetic mice using the programmable platform showed a significant increase in wound closure, re-epithelialization, angiogenesis, and hair growth when compared to conventional topical delivery of therapeutics.

Results & Discussion

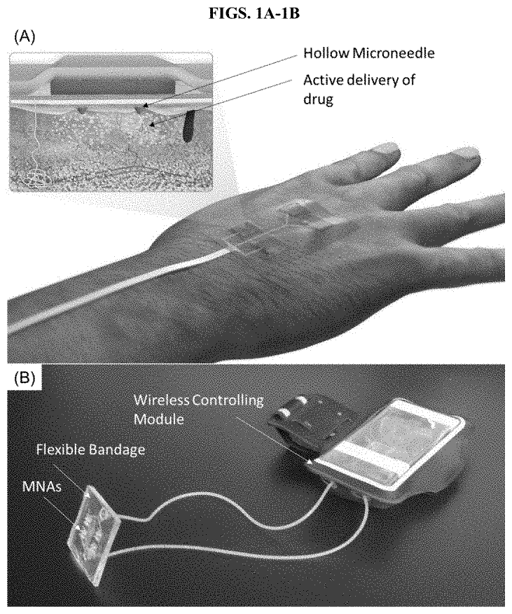

The treatment of chronic wounds is a major medical challenge⁶. The majority of the research in this area have been focused on identifying factors or drugs that can disrupt the dysfunctional physiological processes leading to the occurrence of chronic wounds^{13,14}. Following the recent progress in understanding the biological processes required for proper healing and the pathophysiology of different chronic wounds, it has been suggested that various factors and drugs should be delivered with different temporal profiles^{15,16} Thus, advanced materials and devices have been developed to achieve the proper drug release profiles^{17, 18}. In addition, smart systems have been developed that allow active control over the drug release or even enable the drug release in response to changes in the wound environment^{10,19} However, most of the existing devices have unwanted drug release, which can create side effects or other challenges. For example, the unwanted release of antibiotics can lead to the development of resistance in colonized bacteria, complicating the treatment of potential infections²⁰. Also, almost all the current methods and systems used for controlling drug release in the treatment of wound care are designed for topical delivery of drugs^{11, 11, 21}. This is due to the ease-of-access to the wound site, which allows the localized delivery of therapeutics. However, these systems disregard the pathophysiology of chronic wounds, which is an avascular, or not sufficiently vascularized tissue, covered by a necrotic tissue and a crust. Wound exudates rich with various proteins, inflammatory cytokines, and even pathogens are typically flowing from the inner layers towards the wound surface. Exudates can deactivate and wash away a significant portion of the topically delivered drugs before they reach the targeted cells. We hypothesized that a bandage that allows precise and independent delivery of various drugs or biological factors transdermally could significantly improve the healing rate. To achieve this goal, a programmable smart bandage was designed that utilized hollow MNAs to bypass the wound crust and the necrotic tissue and deliver therapeutics to the deeper layers of wound bed. Active pumping remains one of the most accurate methods for precisely controlling the delivery rate of drug solutions. Therefore, the platform utilized two miniaturized peristaltic pumps that could manipulate minute amounts of two different drugs with independent dosages. A conceptual schematic of the designed platform is shown in FIG. 1A. FIG. 1B presents the prototype of the smart bandage used in this study. MNAs are categorized as minimally invasive delivery tools and are used for the transdermal delivery of therapeutics²².

Recent advances in 3D printing have facilitated the fabrication of miniaturized structures with a suitable resolution without the need for sophisticated cleanroom microfabrication processes. In this study, we used an FDM 3D printer to print hollow MNAs out of a biocompatible resin. The 3D printer was operated by printing support materials to fabricate the high aspect ratio microneedles. The support material of 3D printing has been removed later by dissolving in NaOH solution (FIG. 2A). The 3D printer enabled us to fabricate MNAs with different needle spacing, needle length (0.8-3 mm), base sizes (0.5-1.5 mm), and opening diameters (0.2-0.5 mm). A length of ˜2 mm was selected for MNAs to pass through the crust and part of the necrotic tissue, allowing drug delivery to the deeper layers of the wound. Noting the fact that patients with diabetic ulcers are typically suffering from neuropathy and lack of sensation in their limbs, MNAs insertion will not cause any pain in user patients^{23, 24}. A representative computer model and a micrograph of fabricated MNAs are shown in FIGS. 2A and 2B. The 3D printed MNAs were smooth and sharp enough to penetrate wounded skin (FIG. 2C).

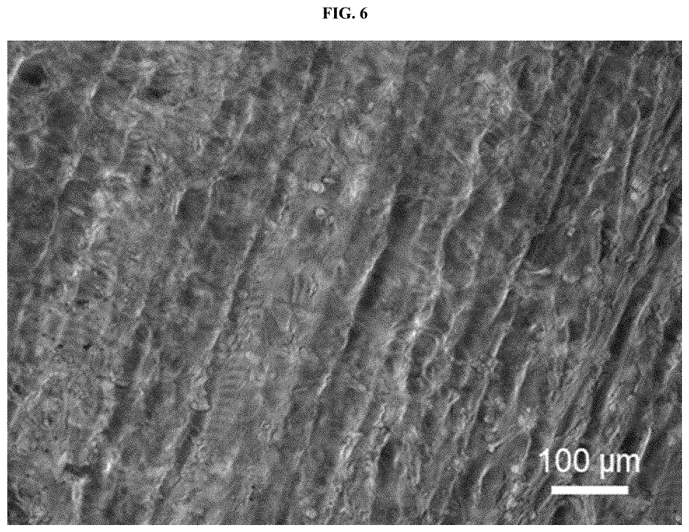

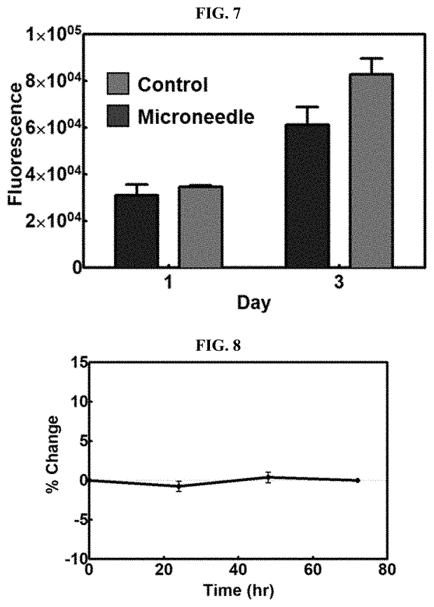

Another interesting aspect of the multi-material 3D printing was the possibility of printing MNAs with rigid resin tips on a flexible polymeric substrate (FIG. 2D). For this reason, needles were printed from a hard resin (Vero Clear, Stratasys, USA), while the backing was printed from another flexible resin (Tango Black, Stratasys, USA). The biocompatibility of the resin-based MNAs were assessed by seeding the MNAs with endothelial cells and performing a live/dead assay in which the live cells appear in green while the dead cells appear in red (FIG. 6). In addition, the effect of the MNAs on the proliferation of the cultured human umbilical vein cells (HUVECs) was investigated by measuring the metabolic activity of cells over time using a PrestoBlue assay. The assay showed that over 3 days of culture, no statistically significant difference in cell proliferation was observed between the HUVECs cultured on MNAs and those cultured in multiwell plates as positive control (FIG. 7). The duration of the experiments was selected based on the intended lifespan of the fabricated bandage. One important factor in the use of MNAs is understanding their degradation rate in wound conditions. To assess that, MNAs were placed in exudate mimicking solutions and their mass was measured over 3 days. No significant change in mass was observed in the MNAs (FIG. 8), suggesting an erosion resistivity of the selected materials.

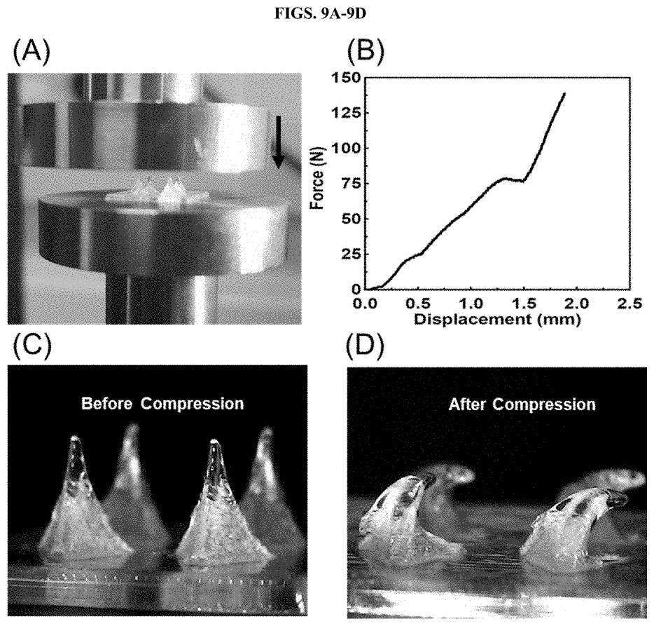

The mechanical properties of MNAs are important in their successful utilization. For example, breakage of the needles during their use can cause inflammation and lead to further complications negatively impacting the healing process. The mechanical properties of the MNAs under compressive loads were assessed by a mechanical tester. In this case, a maximum force of 200 N was applied and the deformation of the MNAs was measured as a function of the applied force. As shown in FIG. 2E and FIG. 9, the MNAs did not break and only were bent under a compressive force of approximately 78 N. A similar test was conducted to assess the penetration and retraction force of the MNAs to and from pig skin. No deformation and breakage were observed upon penetration and removal from the pig skin. (FIG. 2F). Results showed that majority of the MNA penetrated fresh pig skin with less than 2 N force and a full penetration was achieved with about 7 N. The pull-out force for the MNAs were measured to be about 2 N. The trace of full penetration of colored MNAs can be seen on the pig skin by red dots (FIG. 2G).

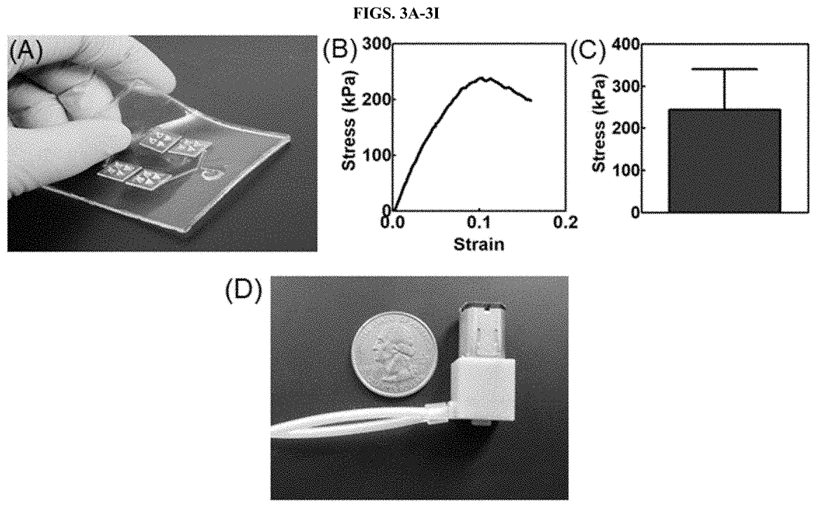

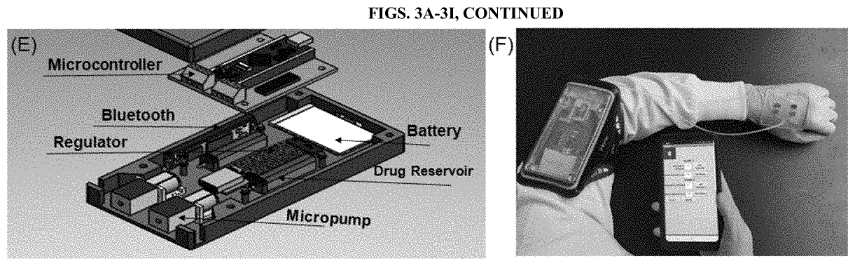

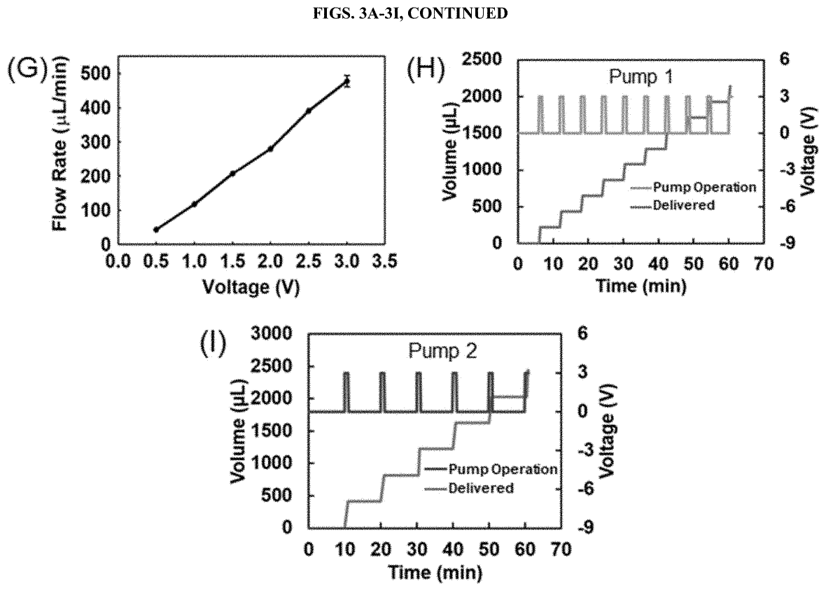

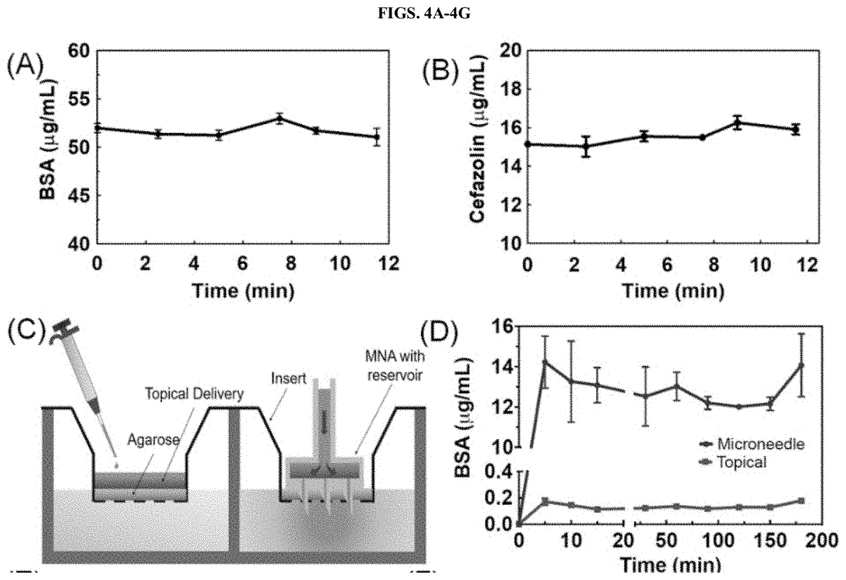

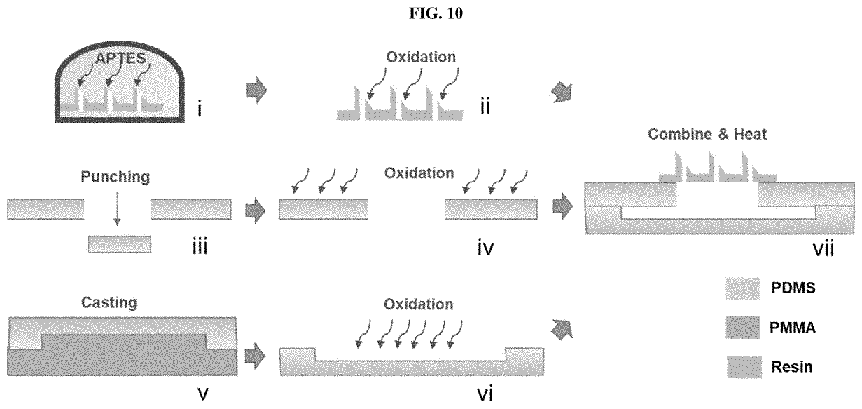

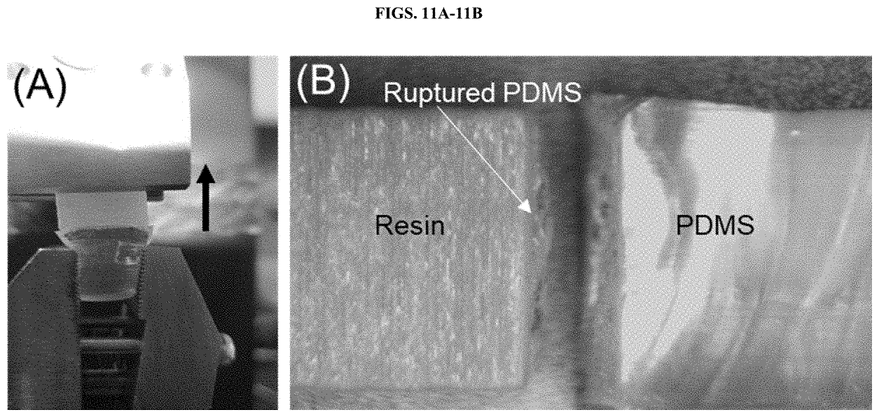

Since many drugs may react with one another, two sets of microchannel arrays, micropumps, and drug reservoirs were utilized to enable the independent handling of solutions. This process enabled complex operation, such as delivering drugs from one set of channels while extracting exudates from another set of microchannels. To ensure the flexibility of the bandage, microchannels were fabricated in a PDMS layer with a thickness of about 1.5 mm. The end of the microchannels were then connected to MNA islands with a footprint of 10 mm×10 mm (FIG. 3A). The use of separated MNA islands further improve the flexibility of the bandage. PDMS was used due to its flexibility, low protein adsorption, biocompatibility, and oxygen permeability. The transparency of the bandage can also facilitate the visual inspection of the wound without the bandage removal. To properly bond the MNA islands to the PDMS substrate, we developed a protocol which is shown in FIG. 10. The bonding strength between the 3D printed resin and PDMS substrate was measured using a peel-off test. The results suggested an average bonding strength of 237 kPa (FIGS. 3B, 3C) for the partial detachment of resin from PDMS substrate (FIG. 11).

To form a fully integrated bandage, peristaltic micropumps (Clark Solutions, MA) were utilized to suffuse the drug solutions into the microchannel arrays and then through the MNA islands. To reduce the bandage operating cost and keep it flexible and light, it was designed with two modules, a disposable module with the MNA islands and microchannel arrays, and a reusable module, which housed the drug reservoirs, micropumps, power source, and electrical circuitry (FIG. 3E). The two modules were connected using two flexible silicon tubes (FIG. 3F). The pumps were controlled by applying a pulse width modulated digital signal from an Arduino Nano microcontroller. The platform could be interfaced with smartphones through Bluetooth. An app was designed to regulate the remote programming of the bandage. The relationship between the applied voltage and the achieved flow rates were determined and is presented in FIG. 3G. The minimum threshold of the pumps was determined to be 0.5V, which resulted in the flow rate of 43.6 μL/min. The data in FIG. 3G was used to generate calibration curves to program the app and precisely control the flow rate. The reproducibility of the generated flow rates was assessed over one hour.

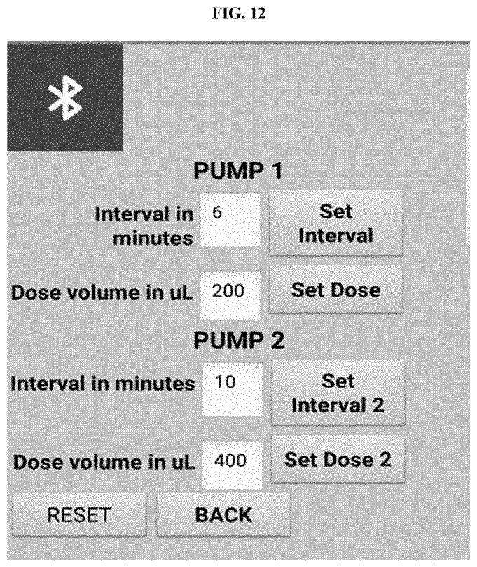

In addition, to test the micropumps response to dynamic actuation, the drivers were programmed to periodically apply constant maximum voltage of 3.0 V. The response of the micropumps to dynamic actuation is shown in FIGS. 3H and 3I for periodic delivery of aqueous solutions. As the voltage value reached zero, the pumping stopped. It can be seen that the pumping process stops as soon as the applied voltage is zeroed. As expected, pump 1 (FIG. 3H) delivered dosage of 200 μL every 6 minutes, while pump 2 (FIG. 3I) delivered 400 μL every 10 min (FIG. 12). It should be noted that the peristaltic pumps could reverse flow direction and generate negative pressure at the MNA islands.

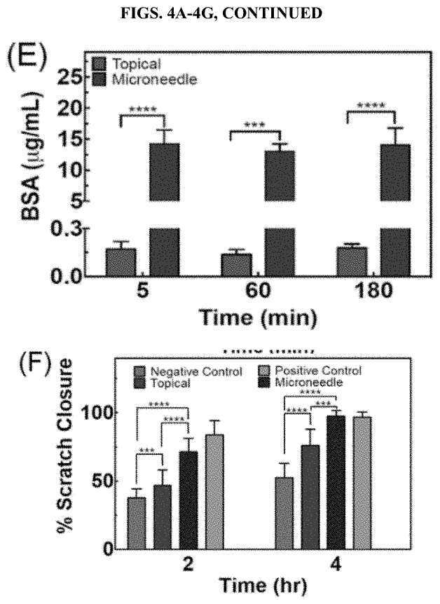

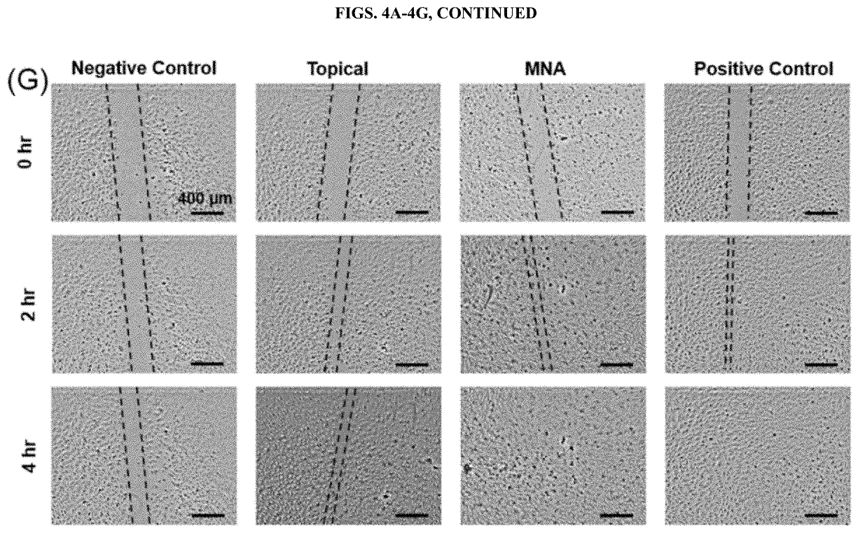

Once the system was characterized, the efficiency of the bandage in drug delivery was evaluated. Initially, the potential drug adsorption to the engineered bandage was to be tested. For this reason, bovine serum albumin (BSA) and cefazolin with 54 ng/mL and 16 μg/mL were loaded into the bandage as model drugs respectively. The solution was then collected at different time points after perfusion through the MNAs and the concentration of the drugs determined using a total protein assay (for BSA) and UV-Vis spectrophotometry (for cefazolin). As shown in FIGS. 4A and 4B, the temporal changes in the drug concentration within the perfused solution was not statistically significant, suggesting the low adsorption of proteins and antibiotics. To assess the importance of the MNAs for the treatment of chronic wounds, we developed an in vitro model resembling the crust and necrotic tissue covering the viable tissue (FIG. 4C). The in vitro model comprised of a cell culture insert with 3 μm pore size coated by ˜2 mm thick 3% (w/v) agarose gel; the interior of the well plate was filled with phosphate buffered saline (PBS) representing the environment of live cells. Agarose gel has been used as skin phantom in several studies and offers porosity and texture similar to skin. To simulate topical drug delivery, 100 μL of drug solution was added on top of the agarose gel. To test the MNA-based delivery, a miniaturized bandage with a diameter of 10 mm (FIG. 13) was fabricated by 3D printing and placed in the agarose gel followed by the delivery of 100 μL of the drug solution using a syringe.

FIGS. 4D and 4E represents the protein release over time, revealing that MNAs enabled a rapid delivery of proteins to the lower chamber representing the environment below the necrotic tissue in comparison to the drug delivered topically (****P<0.0001, ***P<0.001, ****P<0.0001). The results suggest that 70% of the protein was delivered into the bottom chamber using MNAs after 180 minutes, while only 1% of the drug was delivered when BSA solution was added on top of the agarose gel. A similar methodology was applied for the assessment of the effectiveness of VEGF as an angiogenic factor on the culture of human umbilical vein endothelial cells (HUVECs) in vitro. For the experiments, a 200 μm scratch was made in the confluent monolayer culture of HUVECs and VEGF was delivered. Four groups were studied including: 1) 50 ng/mL of VEGF in the culture medium (positive control); 2) no VEGF (negative control); 3) equivalent to 50 ng/mL of culture media delivered topically; and 4) equivalent to 50 ng/mL of culture media delivered using the MNAs.

The data suggest that the group receiving VEGF through MNAs had a migration rate comparable to the positive control group which received VEGF in their culture medium and 100% scratch closure was achieved in 4 hours. The migration and the scratch closure rates in the group receiving VEGF topically was faster than the negative control. However, the migration rate in this group was significantly slower than the two other groups (FIGS. 4F-4G).

Results from the in vitro study suggested the positive effect of MNAs in increasing the drug bioavailability at the site of healthy cells in the wound bed below the wound crust. To further investigate the potential benefits of MNAs in treatment of diabetic and chronic wounds, an animal study was conducted on diabetic mice with cutaneous wounds. Homozygous mice for the diabetes spontaneous mutation (Lepr^{db}) become identifiably obese around 3 to 4 weeks of age. On day 10 to 14 elevations of plasma insulin begin and at four to eight weeks the blood sugar level elevates. With a delayed wound healing and an increased metabolic efficiency this mouse strain serves as a suitable model for chronic wound research.

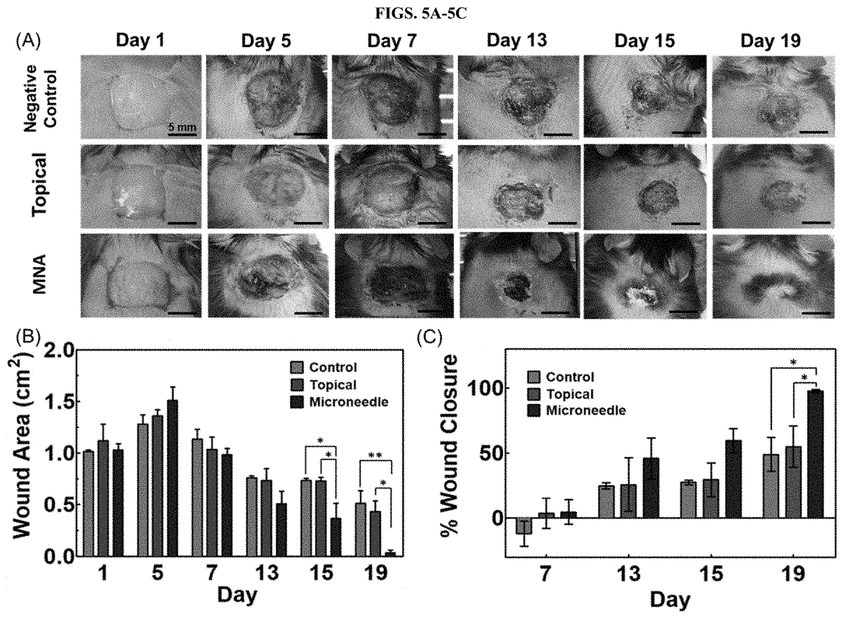

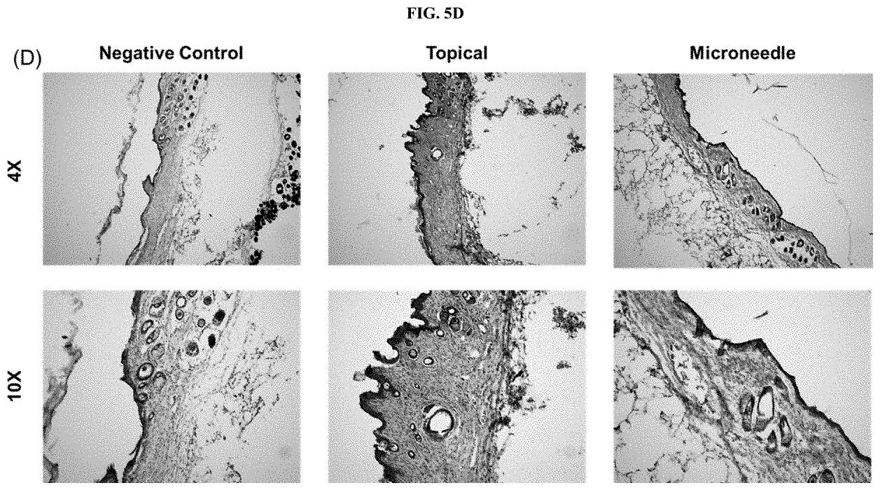

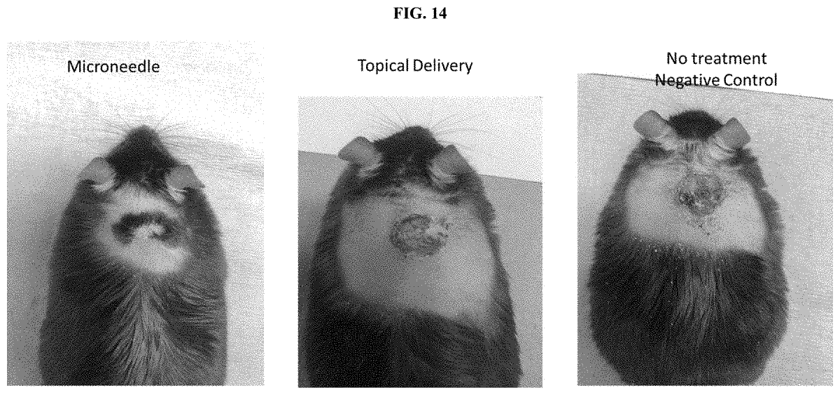

Animals were dived into three study groups including: 1) negative control without receiving any treatment (n=4); 2) topical group, where 100 μL of 500 ng/mL of VEGF solution was pipetted on the wound (n=4); and 3) MNA group in which 100 μL of 500 ng/mL of VEGF solution was delivered using MNAs over 10 min into the wound bed (n=5). On day one, all the mice received a square shaped 1 cm×1 cm full thickness skin cut on their dorsum. The animals were kept untreated for 5 days to allow the formation of wound crust. On days 5 and 7 post-surgery, animals received the planned treatment. T weight of each animal was recorded periodically, and wound areas were assessed every two days and photographed to monitor investigate wound closure (FIG. 5A). The wound area was measured, and the statistical analysis showed significant differences in the wound closure rate in the MNA group compared to the topical and control groups from day 13 post-surgery. On day 15, one-way ANOVA analysis showed significantly different wound closure rate in animals receiving VEGF by MNAs compared to the negative control group (p=0.00916). On day 17 and 19, the wound closure in the MNA group was significantly faster than both topical delivery and negative control groups. At day 19 the average of wound size in the MNAs group decreased to 0.04 cm² with an average of 95% healing rate. At the end of the 19-day study, the animals in the topical delivery group reached about 55% closure, while the negative control group showed about 40% closure. No significant difference was observed in the wound closure rate of animals that received VEGF delivery topically compared to the negative control group (FIGS. 5B-5C). Animals' weight had increased at a rate of 16 percent through the 19-day procedure, but no significant difference was observed between the study groups (FIG. 14).

Another important observation was the significant difference in the quality of wound healing (FIG. 14). In all animals receiving VEGF through MNAs, hair growth in the new tissue was observed. Typically, full thickness injuries result in scarring, which results in lack of hair growth. However, in our MNA group, no evidence of scaring was observed at the end of the experiments. The observation of hair growth due to VEGF delivery might be due to better wound vascularization and in growth and differentiation of the new tissue. It should be noted that the positive role of VEGF on new hair growth has been suggested previously. However, more detailed experiments are required to understand the mechanism stimulating hair growth. Similar to many other studies, no hair growth was observed in the wound area for the animals in both topical delivery and negative control groups.

At the junction of the surgical wound and adjacent skin is the healing site, which is moderately thickened by granulation tissue. The granulation tissue in the surgical site is associated with alopecia, deposition of collagen, and neovascularization. There is a junctional area of granulation tissue bordering the normal haired skin, which contains a few pilosebaceous units. The appearance of the junctional skin and haired skin along the periphery is indistinguishable among all of the mice, indicating that the shrunken wound sizes in the treatment groups has followed orderly wound healing with regrowth of all dermal, follicular, and epidermal elements.

Conclusion

The treatment of chronic wounds has remained a major medical challenge and devices that can reduce the need for frequent visit to medical facilities while enabling the possibility of releasing different drug independently would be valuable. One important question in wound care is if the topical delivery of drugs is suitable for the treatment of exuding chronic wounds covered with crust and necrotic tissue. Here, we developed a programmable platform with the capability of actively controlling the release profile of multiple drugs. The platform benefits from multiple miniaturized pumps wirelessly controlled through a smart phone application. All the electronics were integrated into a smart phone size module that could be reused. To improve the bioavailability of drugs and active compounds at deeper layer of the tissue, the bandage was equipped by islands of MNAs. The effectiveness of the MNAs in transferring the active compounds through the wound crust and necrotic tissues were successfully demonstrated in vitro. The platform was then utilized for the delivery of VEGF for the treatment of 5 days old full thickness skin injury in diabetic mice. The results showed a significant difference in wound closure and a fundamental difference in healing quality. The animals received VEGF through the MNAs shown signs of complete healing and lack of scar formation. Our data suggested that in addition to the used active compounds and their release profile, the point of delivery should be considered in treating chronic wounds. As a result, the use of MNA-based patches for wound care could be an effective paradigm shift from the current methods used in clinical wound care practices.

Materials & Methods

Materials: Materials and reagents including NaOH, cefazolin, (3-Aminopropyl) triethoxysilane (APTES), HUVEC culture media, BCA Protein Assay kit 23225, Human VEGF 165, FBS, Geltrex matric, and PBS were purchased from Sigma-Aldrich (MO, USA). Polydimethylsiloxane (PDMS) was purchased from Dow (MI, USA). 3D printing resins were obtained from Stratasys (MN, USA). Recombinant Mouse VEGF (VEGF 164) was purchased from Biolegend (CA, USA) and Anti-CD31 antibody ab28364 was obtained from Abcam (Cambridge, Mass., USA).

MNA Fabrication and their Integration to the Bandage: MNAs were fabricated using a FDM 3D printer (Objet 500 Connex3, Stratasys, USA) out of VeroClear™ RGD810 (Stratasys, MN, USA), which is a transparent, rigid, and nearly colorless resin for 3D printing. After 3D printing of the MNAs, the support material was removed using 3% (w/v) NaOH solution in an ultrasonic bath. Micromolding of PDMS was used to fabricate a flexible bandage with incorporated microchannel patterns. To permanently bond MNAs to the PDMS bandage, the pieces were silanized by APTES overnight. Both surfaces of the MNAs and bandage were then activated using a plasma cleaner (Harrick Plasma, USA) in high mode for 15 seconds. The samples were interfaced and kept under pressure (using a 1 lb. weight) at 80° C. for 2 hours to complete the bonding. The bonding strength between resin and PDMS was measured using a mechanical tester (CellScale Univert). Cubic samples of PDMS and Resin (1 cm³) were fabricated and bonded together as described above. The force required for the detachment of the pieces were measured. The experiments were done in 4 replicates.

The mechanical strength of the microneedles was measured under a compressive load using the mechanical tester. MNAs were glued on the lower grip of the mechanical tester. Then, the upper jaw was brought in contact with the array and compressive load was applied by moving the jaws at the rate of 0.092 mm/s. The experiments were done in triplicate.

The characterization of MNAs penetration through pig skin was evaluated using the mechanical tester. To this means, MNAs were glued to the upper jaw of the mechanical tester and a 3 cm×3 cm fresh pig skin was placed on the lower jaw. The jaws were moved until the MNAs were touching the skin and the device was run under the compression mode at the rate of 0.27 mm/s. After penetration, the samples were left at rest for 1 minute, and then they were retracted by running the mechanical tester under the tensile loading mode (at the same rate of insertion) to measure the pull-out force.

In order to evaluate the possible degradation of microneedles in situ, a wound exudate-mimicking solution was generated according to previous studies. The solution contained 0.368 g CaCl₂ and 8.29 g NaCl (pH 8.2), which were dissolved into 1000 mL of distilled water and stirred for 10 min. Twelve MNAs were prepared as previously described, rinsed with distilled water, and fully submerged in 3 mL of exudate mimicking solution at 37° C. On days 1, 3, 5, and 7, the weight of the dry weight of MNAs were measured to determine the rate of mass loss.

Integration of Pumps and Electronics: A RPQ1 peristaltic micropump was chosen to precisely control drug administration given its ability to manipulate small quantities of medications. As different drugs or biological factors are needed at various stages of wound healing, the bandage was engineered to handle two different drugs independently. In order to run two pumps bidirectionally, an L293D half H-bridge (LCSC, USA) was selected as the driver. L293D, an Arduino Nano Clone (Elegoo, USA) was chosen as the microcontroller for providing 5V logic output. To provide wireless communication, a HC-05 Bluetooth module (Hiletgo, China) was selected. A custom-built PCB (JLC PCB, USA) was used for the assembly of various components. The electronics, micropumps, drug reservoirs were housed in a 3D printed box (1 cm×10 cm×15 cm). The fabricated module was placed within an armband and was connected to the bandage with the integrated MNAs using flexible silicon micro tubing.

For maintaining proper function during administration, a simple code was written to apply a pulse width modulated (PWM) signal from the Arduino pins. The code allowed the PWM value to be adjusted from 0 to 255 to simulate a variable voltage input to the pumps. This code was used to correlate the effective voltage input level to the volume of fluid dispensed. Next, an Android smartphone application was programmed using the drag-and-drop programming of MIT App Inventor (available at appinventor.mit.edu on the World Wide Web). This app allows the user to set pump dosing volume, either manually or on a timer, with the two pumps able to deliver different doses at different times. This creates the opportunity to provide two different drugs to the wound. To complete the software system, an Arduino program was written to read the input transmitted from the app via Bluetooth. The program then takes the user input and converts it to milliseconds; however, the volume input was converted to the time which the pump must run at full speed to deliver the desired dose, the calculation was done based on the data from the correlation between micro pump and its flow rate.

Drug Delivery Characterization: The potential protein and drug adsorption by the bandage, tubing, and MNAs were investigated by perfusing a solution with known concentrations of cefazolin salt and BSA. 150 μL of the delivered solution was collected at different time points and the concentrations of cefazolin and BSA were determined by measuring the solution absorbance for cefazolin and BCA protein assay kit for BSA.

To develop a method for mimicking the crust and necrotic tissue covering chronic wounds, 12-well cell culture inserts were used and covered with 150 mL of 3% (w/v) agarose solution (1.5% w/v). Once the agarose gel was solidified, the insert was placed in 12-well plates, the bottom chamber was filled with 2 mL of PBS, and the active compounds were delivered. Similar techniques were applied for scratch assay with seeding HUVECs in the bottom of chamber covered by 2 mL basal media. To mimic topical delivery, the active compounds (BSA or VEGF) were directly poured on top of the agarose gel, while in the case of MNA-based delivery MNAs with the diameter of 9 mm were inserted into the gel and perfusion was carried out at a rate of 7.5 μL/sec.

In the case of BSA, to measure its concentration within the solution inside the wells, at each time point 450 μL of the solution was removed and replaced by fresh PBS solution. The concentration of BSA was measured using a BCA total protein assay kit as per the manufacturer's protocol.

Cell Culture and In Vitro Experiments: HUVECs were purchased from Sigma-Aldrich and were cultured in the endothelial growth media (Sigma-Aldrich) and were used up to passage 6. To assess the biocompatibility of the MNAs, they were coated with Geltrex diluted 1:20 in media at 37° C. The MNAs were seeded with about 20,000 cells and after 1 day their viability was assessed using a Live/Dead™ Viability/Cytotoxicity Kit (Invitrogen, state) as per the protocol recommended by the manufacturer. In brief, samples were incubated with a mixture of 2 μl/mL ethidium homodimer and 0.5 μl/mL calcein-AM in PBS for 10 minutes at 37° C. The samples were imaged using a Zeiss Observer fluorescence microscope, where live cells appeared as green while the dead cells appeared in red. The effect of MNAs on cellular proliferation was assessed indirectly by measuring the metabolic activity of cells using a PrestoBlue assay (Invitrogen, state) as per manufacturer recommended protocol. For this reason, 15,000 of cells were cultured in 24-well plates and MNAs of 7 mm×7 mm were interfaced with them. On days 1 and 3, the PrestoBlue reagent mixed with media (1:10 ratio) was added and the cultures were incubated for 1 hour. After the incubation, the fluorescence intensity of the solution was measured using a Cytation 5 UV-Vis spectrophotometer (Biotek, State). Four samples were used, and the growth rate was compared to cells cultured in multiwell plates without being contacted to MNAs.

To assess the effectiveness of the released factors, a standard scratch assay was conducted. Wells of a 12-well plate were coated by Geltrex. After aspirating the Geltrex from the wells, 30,000 cells per well were seeded and cultured until a confluent monolayer formed in each well. The cells were cultured in basal media supplemented with 2% FBS for 6 hours. prior to the experiments. On the day of experiments, an approximately 200 μm wide scratch was formed using a 200 μL micropipette tip. The samples were divided into 4 groups having 4 replicates for each group: 1) positive control receiving a VEGF supplement of 50 ng/mL in their culture media; 2) negative control cultured without receiving VEGF supplement; 3) MNAs group receiving VEGF containing solution the same as the positive control through the MNAs placed in tissue culture inserted filled with agarose gel; 4) topical group VEGF containing solution the same as the positive control through added on top the agarose gel within the tissue culture inserts. Fixed points of each well (two points per well) were imaged using a Nikon Ti inverted microscope after 0, 2, and 4 hours after each treatment application. A total of 4 samples per condition was tested.

Animal Studies: All animal procedures were reviewed and approved by the Institutional Animal Care and Use Committee (IACUC) of the University of Nebraska, Lincoln. Five weeks old B6.BKS(D)-Lepr^{db}/J mice were purchased from the Jackson Laboratory (Bar Harbor, Me.) weighted between 34-43 grams and accommodated in University of Nebraska, Life Science, animal facility for one week before the wound injury procedure. The animals were on special food diet during the entire research period. Sex-matched controlled mice divided into three study groups including: (1) negative control (no treatment) (n=4); (2) test group receiving VEGF topically; and (3) test group receiving VEGF through MNAs delivery (n=5) and received two sessions of VEGF (Vascular endothelial growth factor) totaling n=13.

In the day 1, all animals were anesthetized by 5% of isoflurane using anesthesia system, (VetFlo, Kent Scientific, Torrington, Conn.) via a nose cone. The hair on the dorsal region of animals was shaved using an electric razor. The skin was sterilized, and a full thickness skin cut of 1 cm×1 cm was created. The wounds were covered with a regular bandage. All mice placed back in separate cages individually after recovering from anesthesia. Animals were monitored for wound condition, vitals and wellbeing, and weight gain/loss trends daily.

To simulate the real wound conditions covered by crust and necrotic tissues, treatment start points were on day 5 by which a complete crust was formed on the wounds. Murine VEGF was dissolved in PBS (phosphate buffered saline) containing 0.1% (w/v) BSA (bovine serum albumin) at the concentration of 500 ng/mL used as animal vascular permeability factor. In the topical delivery group, 100 μl of the solution was poured topically using a pipette on the top of the wound and the animals were let to sit for about 10 minutes, the wound was then covered by a fresh dressing. In MNAs delivery group, 100 μl of the solution was delivered using the MNAs and after 10 min, the microneedles were removed and the wound was covered by a fresh dressing. Animals of negative control received no treatment. Similar procedure was followed on day 7 to deliver VEGF for a second round. Wounds were inspected every 2 days and the bandage was changed and a picture of the wound was taken using a digital camera.

On day 19, all animals were sacrificed using 20% CO₂ volume displacement (flow rate cage volume/per minute) poisoning overdose and wound tissue was harvested and fixed in using 4% paraformaldehyde (PFA).

Histology: Frozen sections of haired skin were stored in a −80° C. ultralow freezer prior to sectioning on a cryotome. The sections of skin were cut 10 μm thick using a Thermo Scientific CryoStar NX50, routinely stained with hematoxylin and eosin on a Leica ST5020 H&E stainer, and coverslipped with a Leica CV5030 coverslipper. A veterinary anatomic pathologist who is board certified by the American College of Veterinary Pathologists performed all histological evaluations. Immunohistochemistry was done manually using Anti-CD31, rabbit host species (ab28364, Abcam, MA, USA).

### References

1 Frykberg, R. G. & Banks, J. Challenges in the Treatment of Chronic Wounds. Advances in Wound Care 4, 560-582, doi:10.1089/wound.2015.0635 (2015).
2 Werdin, F., Tenenhaus, M. & Rennekampff, H.-O. J. T. L. Chronic wound care. 372, 1860-1862 (2008).
3 Sen, C. K. et al. Human skin wounds: A major and snowballing threat to public health and the economy. Wound Repair and Regeneration 17, 763-771, doi:10.1111/j.1524-475X.2009.00543.x (2009).
4 Fife, C. E., Carter, M. J., Walker, D. & Thomson, B. J. W. Wound care outcomes and associated cost among patients treated in US outpatient wound centers: data from the US wound registry. 24, 10 (2012).
5 Guo, S. & DiPietro, L. A. Factors Affecting Wound Healing. Journal of Dental Research 89, 219-229, doi:10.1177/0022034509359125 (2010).
6 Saghazadeh, S. et al. Drug delivery systems and materials for wound healing applications. Advanced Drug Delivery Reviews 127, 138-166, (2018).
7 Guo, J., Dardik, A., Fang, K., Huang, R. & Gu, Y. Meta-analysis on the treatment of diabetic foot ulcers with autologous stem cells. Stem Cell Research & Therapy 8, 228, doi:10.1186/s13287-017-0683-2 (2017).
8 Mustoe, T. A., O'Shaughnessy, K. & Kloeters, O. Chronic Wound Pathogenesis and Current Treatment Strategies: A Unifying Hypothesis. 117, 35S-41S, doi:10.1097/01.prs.0000225431.63010.1b (2006).
9 Demidova-Rice, T. N., Hamblin, M. R. & Herman, I. M. Acute and impaired wound healing: pathophysiology and current methods for drug delivery, part 1: normal and chronic wounds: biology, causes, and approaches to care. Advances in skin & wound care 25, 304-314, doi:10.1097/01.ASW.0000416006.55218.d0 (2012).
10 Derakhshandeh, H., Kashaf, S. S., Aghabaglou, F., Ghanavati, I. O. & Tamayol, A. Smart Bandages: The Future of Wound Care. Trends in Biotechnology 36, 1259-1274, (2018).
11 Kiaee, G., Mostafalu, P., Samandari, M. & Sonkusale, S. A pH-Mediated Electronic Wound Dressing for Controlled Drug Delivery. Advanced Healthcare Materials 7, 1800396, doi:10.1002/adhm.201800396 (2018).
12 Mostafalu, P. et al. A Textile Dressing for Temporal and Dosage Controlled Drug Delivery. Advanced Functional Materials 27, 1702399, doi:10.1002/adfm.201702399 (2017).
13 Harding, K. G., Morris, H. L. & Patel, G. K. Healing chronic wounds. BMJ 324, 160, doi:10.1136/bmj.324.7330.160 (2002).
14 Han, G. & Ceilley, R. Chronic Wound Healing: A Review of Current Management and Treatments. Advances in Therapy 34, 599-610, doi:10.1007/s12325-017-0478-y (2017).
15 Leaper, D. J. et al. Extending the TIME concept: what have we learned in the past 10 years?*. International Wound Journal 9, 1-19, doi:10.1111/j.1742-481X.2012.01097.x (2012).
16 Falanga, V. The chronic wound: impaired healing and solutions in the context of wound bed preparation. Blood Cells, Molecules, and Diseases 32, 88-94, (2004).
17 Chen, F.-M., Zhang, M. & Wu, Z.-F. Toward delivery of multiple growth factors in tissue engineering. Biomaterials 31, 6279-6308, (2010).
18 Lee, K., Silva Eduardo, A. & Mooney David, J. Growth factor delivery-based tissue engineering: general approaches and a review of recent developments. Journal of The Royal Society Interface 8, 153-170, doi:10.1098/rsif.2010.0223 (2011).
19 Punjiya, M., Mostafalu, P. & Sonkusale, S. in 2017 IEEE 60th International Midwest Symposium on Circuits and Systems (MWSCAS). 495-498.
20 Tilelli, J. A., Smith, K. M., Pettignano, R. J. P. T. J. o. H. P. & Therapy, D. Life-Threatening Bradyarrhythmia After Massive Azithromycin Overdose. 26, 147-150 (2006).
21 Tamayol, A. et al. Biodegradable elastic nanofibrous platforms with integrated flexible heaters for on-demand drug delivery. Scientific Reports 7, 9220, doi:10.1038/s41598-017-04749-8 (2017).
22 van der Maaden, K., Jiskoot, W. & Bouwstra, J. Microneedle technologies for (trans)dermal drug and vaccine delivery. Journal of Controlled Release 161, 645-655, (2012).
23 Rinkel, W. D. et al. Grading the loss of sensation in diabetic patients: A psychometric evaluation of the rotterdam diabetic foot study test battery. Muscle & Nerve 58, 559-565, doi:10.1002/mus.26192 (2018).
24. Ahmad, J. The diabetic foot. Diabetes & Metabolic Syndrome: Clinical Research & Reviews 10, 48-60, (2016).

### Example 2—Intradermal VEGF Delivery Accelerates Wound Healing

Skin injuries are one of the major healthcare challenges in the United States¹. Different wounds have been estimated to affect more than eight million Americans and to cost the US economy more than $28-96 billion annually^{2,3}. Increases in the aging population and incidence of diabetes raise the prevalence of chronic wounds each year^{4,5}. Normal wound healing is an orchestrated cascade of physiological events which involves hemostasis, recruitment of immune cells to remove pathogens and modulate inflammation, migration of early responder cells and depositing of a temporary extracellular matrix (ECM), the ingrowth of blood vessels and epithelial cells, removal of immune cells from the injury site, remodeling of the ECM, and closure of the wound^{6,7}. However, in many conditions, patients suffer from “under healing” (which causes chronic wounds) or “over healing” (which results in scarring⁸). In chronic wounds, the healing cascade is locked in a state of pathologically elevated inflammation⁹⁻¹¹. On the other hand, scarring is a result of persistent wound contraction and ECM remodeling even after wound closure^{8,12}. Impaired vascularization can induce or magnify these conditions by interrupting the normal healing cascade. Lack of proper vascular network can result in limited tissue oxygenation and metabolic support, insufficient immune response to infection, dysregulated cell infiltration, and delayed cellular response to physiological signals¹³.

Increasing evidence has demonstrated the benefits of various growth factors, cytokines and peptides for improving wound healing¹⁴⁻¹⁷. Given the crucial role of vascularization in wound healing, vascular endothelial growth factor (VEGF) is an interesting candidate to improve the healing process¹⁸⁻²⁰. VEGF plays an essential role in angiogenesis, vascular and lymphatic growth, and their patterning^{21,22}. Being produced by many cell types including platelets, fibroblasts, and immune cells especially under hypoxic conditions, VEGF stimulates vasodilation to facilitate the infiltration of immune cells, and endothelial cell growth and migration essential for vascularization of the wound bed²². VEGF is also suggested to promote collagen deposition and epithelization²³, as well as the formation of hair follicles²⁴. However, endogenous VEGF in many pathological situations is insufficient for stimulation of the endothelial cells and induction of wound healing²⁵. Thus, researchers have delivered exogenous VEGF to stimulate proper vascularization of the wound bed. While topical delivery of VEGF has shown some benefits, its effectiveness was limited in enhancing the rate and quality of wound healing^{26,27}. We suspect that the limited impact of topical delivery of VEGF might be due to its inefficient penetration into the wound bed. When VEGF is applied topically, it needs to penetrate the wound eschar first and subsequently travel through the granulation tissue to reach endothelial cells. Particularly in chronic wounds, the continuous production of wound exudate rich with various enzymes and cytokines can further lower the availability of VEGF at the targeted point^{7,28}.

To enhance the effectiveness of VEGF delivery and consequently promote the rate and quality of wound healing, here we explore the potential benefit of two different mechanical systems, hollow miniaturized needle array (MNA) and liquid jet injector (LJI), for intradermal delivery of VEGF, offering different spatial distribution of drug in wound bed. The distribution of drugs delivered using these systems is assessed and optimized in vitro. The systems are then used for intradermal VEGF delivery to wound beds in diabetic mice, and the results are compared to the healing rate of animals receiving VEGF topically and no treatment. To evaluate the potential of the strategy in wound care practice, the effects of intradermal VEGF delivery using MNAs on the rate and quality of cutaneous wound healing is investigated in a swine model.

Results

In Vitro Evaluation of Intradermal Delivery Systems

The treatment of wounds in a timely manner for recovery of the functional tissue is a major medical challenge, particularly in chronic wounds. Various drugs, biological factors, and biomaterials have been identified²⁵. These therapeutics are first tested in vitro on cultured cell and then in vivo. However, to date, clinical translation of these approaches showed limited effectiveness. A serious problem in almost all of these investigations has been the inefficient delivery of therapeutic agents²⁹. This is of course the result of limited and delayed diffusion of higher molecular weight compounds, the presence of physical barriers such as eschar, opposite exudate flow, and harsh microenvironment rich with various proteases degrading the bioactive factors¹⁰.

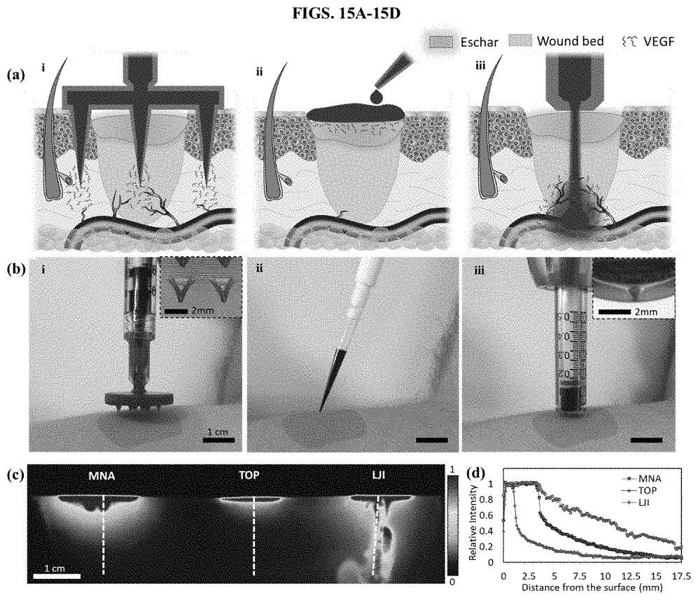

Here, the effect of controlled spatial distribution of VEGF in the wound bed on the rate and quality of wound healing was evaluated (FIGS. 15A-15D). Different intradermal delivery tools namely MNAs and LJI were explored aiming to reduce the required traveling distance of VEGF, bypass eschar from the delivery path, control the distribution area, and consequently stimulate endothelial cells such that they support proper vascularization essential for tissue healing (FIG. 15A).

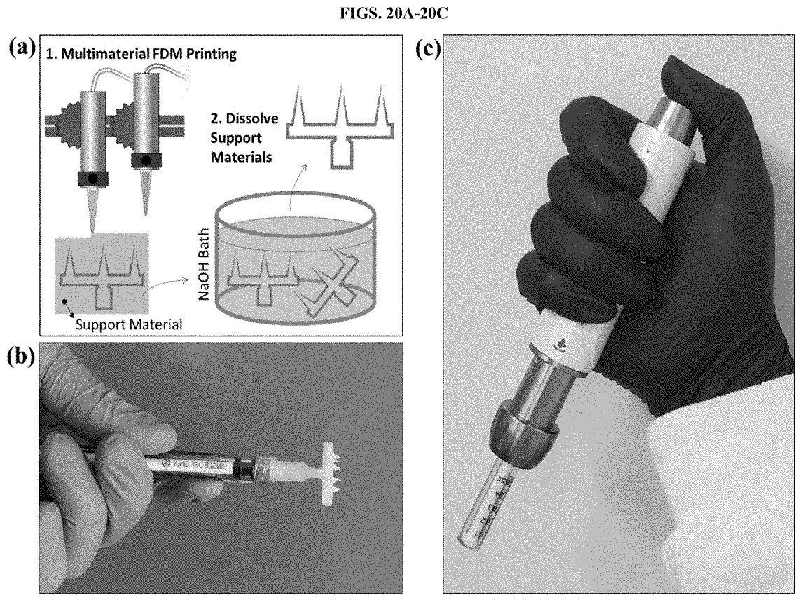

FIG. 15B demonstrates different tools employed here for administration of the therapeutics. Micro- and miniaturized needle arrays have been commonly implemented for painless transdermal drug delivery, though their application in wound care has been limited^{7,30}. These structures have been fabricated from a wide variety of materials and by different methods such as molding, laser ablation, photolithography, and recently 3D printing^{31,32}. Following our recent study¹³, a fused deposition modeling (FDM) approach was utilized for 3D printing of hollow MNAs (FIGS. 20A-20C; FIG. 15B-i). This robust process allows fabricating MNAs with desired needle spacing, needle length, base sizes, and opening dimensions. A biocompatible resin was implemented to fabricate 3 mm-long MNAs in this study, corresponding to the thickness of combined epidermis and dermis in humans³³, as it was expected to be long enough to penetrate deep into the eschar, allowing drug distribution into the wound bed. LJI shown in FIG. 15B-iii, also known as needle-free drug delivery system, is another tool for subcutaneous delivery of drugs, vaccines, and cosmetic gels^{34,35}. Despite their wide applications, LJIs have rarely been used for wound healing³⁶.

In order to study the spatial distribution of delivered drug using MNAs and LJI, and compare it with that of topical administration, 100 μL of 5 mg/mL rhodamine B solution was delivered into a 3% (w/v) agarose gel block using each delivery strategy. Agarose and other agar-based hydrogels have been widely used as skin models in previous studies^{37,38}. FIG. 15C represents the fluorescent intensity map of distributed rhodamine B after 30 min in the agarose gel block. The intensity is also plotted quantitatively against the depth of penetration (FIG. 15C). Results demonstrated different distribution profiles for the solution delivered by MNAs, LJI or topically (TOP). Compared to topical administration, MNAs delivered the solution deeper into the skin model while showing a relatively uniform distribution area. In topical delivery, the intensity of the dye promptly decreased after only ˜1 mm. This value is attributed to the diffusion of the dye into the gel structure. Considering the presence of eschar and the opposite flow of exudates particularly in chronic wounds, the penetration of bio-reagents administrated topically is expected to be even less than 1 mm. These results clarify the importance of intradermal systems such as MNAs in transporting and delivering the drugs deeper into the wound bed.

The other intradermal delivery system, LJI resulted in the deepest penetration of the dye, though the spatial distribution was non-uniform. LJI creates a burst pressure in the order of hundreds of kPa, injecting the solution through a micro-nozzle (FIG. 15B-iii)^{39,40}. This burst injection generates a high-speed, high Reynolds number, turbulent flow causing the failure of the tissue surface, puncturing it to deliver solutions deep into the lower layers of the skin model, while the propagation of the failure depends on the mechanical properties of the structure and can be non-uniform^{39,40}. Overall, the results suggest that unlike topical delivery that shows limited penetration into the skin model, intradermal injections using LJI enables the delivery of compounds into deeper layers of the skin, while MNAs provide a larger and more uniform distribution area with a reasonable depth of delivery.

The intradermal drug delivery kinetics were further evaluated by developing an in vitro model mimicking the structure of wounds in which eschar and granulation tissue separates the endothelial cells from the outside environment. This model, illustrated schematically in FIG. 2a, consisted of a cell culture insert with a porous membrane covered by agarose (3% w/v) with ˜2 mm thickness, and the bottom well represented the environment of endothelial cells.

To compare the delivery rate of therapeutics using the described technologies, three different molecule types were tested: bovine serum albumin (BSA) as a model protein (with a molecular weight of ˜66 kDa, which is comparable to VEGF with a ˜46 kDa molecular weight⁴¹), cefazolin as an example antibiotic, and rhodamine B as an example of a neutrally charged small molecule. FIG. 16B shows the delivery rate of proteins into the chamber that mimics the wound bed. As expected, the results suggested that BSA concentration in the lower chamber delivered using LJI and MNAs was higher than the value when delivered topically.

The overall efficiency of protein delivery after 180 min was ˜70% for MNAs and ˜80% for LJI, while only ˜25% of the BSA passed through the agarose coated insert when it was applied topically. In a similar experiment, more than 90% of cefazolin was delivered into the bottom chamber using the LJI, ˜65% using the MNAs, and ˜25% when delivered topically (FIG. 16C). Delivery of rhodamine B also indicated a similar trend (FIG. 16D): 75% of the solution was delivered to the bottom chamber in the LJI group, ˜60% for the MNAs, and less than 15% for the TOP delivery after 180 min. These results supported our initial hypothesis that compounds delivered topically to wounds covered with eschar have limited local bioavailability in cellular environment, while LJI and MNA-based delivery can improve drug penetration and distribution. As a result, the delivery of drugs through LJI and MNAs is expected to be more effective in induction of healing.

Intradermal VEGF Delivery Improves Diabetic Wound Healing

VEGF has been shown to affect endothelial cells' mobility and act as an angiogenic factor in neo-angiogenesis by promoting their proliferation and migration^{42,43}. To evaluate the effectiveness of intradermal VEGF delivery in the improvement of wound healing, in vivo investigations were conducted on diabetic mice with full-thickness skin wounds. Homozygous mice with the diabetes spontaneous mutation (Lepr^{db}) demonstrating an elevated blood sugar level were used in this study. This mouse strain can be considered as a proper model for investigating delayed wound healing¹³.

On the day of surgery, mice received a full thickness (1×1 cm²) skin wound on their dorsum. Mice with the full thickness (1×1 cm²) wound on their dorsum were kept untreated for 5 days to allow eschar formation. Animals received treatment via three different delivery methods on days 5 and 7 post-surgery. Six groups of in vivo VEGF assessment: 1) negative control without receiving any VEGF treatment (NGT control, n=5); 2) test group receiving 100 μL of 500 ng/mL of VEGF solution was pipetted on the wound topically (TOP VEGF, n=5); 3) test group receiving 100 μL of 500 ng/mL of VEGF solution through MNAs (MNA VEGF, n=5); 4) test group receiving 100 μL of 500 ng/mL of VEGF solution through LJI (LJI VEGF, n=5); 5) second control group receiving 100 μL of PBS through MNAs (MNA PBS, n=3); and 6) third control group receiving 100 μL of PBS through LJI (LJI PBS, n=3). Similar to in vitro experiments, to minimize the potential injury upon the delivery of therapeutics intradermally using LJI, a layer of agarose was placed between the wound and the nozzle. Despite this precaution, some of the animals died quickly post injection due to internal trauma, which were excluded from the analysis.

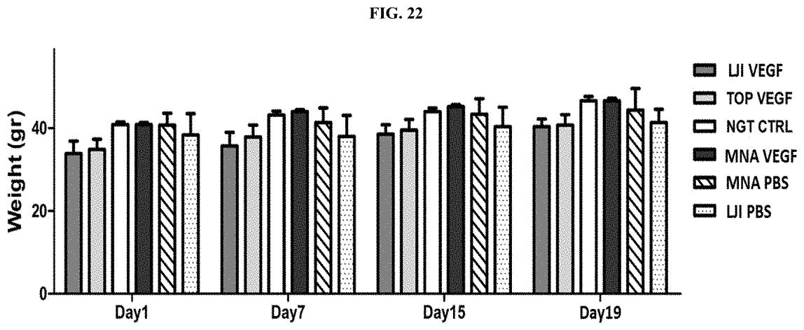

Animals' weight was recorded periodically, and the wound area was assessed every two days, and photographs were captured to investigate wound closure (FIG. 17A). However, since the wounds were covered by scab, an accurate quantification of the wound closure was not feasible and, in some cases, the natural detachment of the scab revealed that wound was partially healed underneath. Consequently, quantification of wound closure area resulted in moderate variation in each group. The statistical analysis showed significant differences in the wound closure rate in the groups received VEGF via MNA and LJI compared to the topical delivery of VEGF and different no VEGF control groups in day 19 post-surgery. On day 15, statistical analysis showed significantly different wound closure rate in animals receiving VEGF by MNAs compared to negative control group and animals received PBS via MNAs. On day 19, wound in the MNA and LJI groups was significantly more healed as compared to topical delivery and negative control groups. The average wound size in the MNAs group at the end of the study decreased to 0.04 cm² with an average of 96% healing rate, and an average of 81% healing rate was seen in the LJI treated animals. At the end of the 20-day study, the animals in the topical delivery group reached about 66% closure, while the negative control group showed about 42% closure. Overall, groups receiving intradermal VEGF delivery demonstrated faster healing compared to those with topical administration as well as control groups (FIG. 17B). The results support the hypothesis that intradermal delivery can improve the local bioavailability of the drugs in wound bed and enhance the healing of chronic wounds. No significant difference was observed in the wound closure rate of animals that received VEGF delivery topically compared to negative control groups with no treatment or PBS treatment via MNA and LJI. Animals' weight had increased at a rate of 16 percent through the 20-day procedure, but no significant difference was observed between the study groups (FIG. 22).

To further investigate the effect of intradermal VEGF delivery on the diabetic wounds healing, histological analysis was performed on the samples harvested from the mice at the wound site on day 19 (FIG. 17C; lower magnification pictures of all groups is depicted in FIGS. 23A-23B). Because the histological analysis is not affected by the presence of scab, it can provide valuable information on the wound healing status. Regardless of the treatment group, all mice demonstrated histological features associated with wound healing, but with significant differences between the groups. As expected, because healing naturally progresses from the periphery of a lesion, the periphery of the wounds was in a more advanced state of healing and more closely resembled normal skin than the central regions. Granulation tissue was observed in the periphery of the wounds, maturing into fibrous tissue and embedded with pilosebaceous units.

At the central region of the wounds, there were significant differences between the groups, indicative of different rate and quality of wound healing. The histological features in the groups receiving VEGF intradermally using different tools supported our hypothesis. These animals, histologically, had more advanced wound healing characteristics, showing ingrowth of dermal, follicular, and epidermal elements through the granulation tissue, whereas the control and TOP-administrated VEGF groups contained a bed of granulation tissue with a partially healed center and were lacking those elements, as shown in FIG. 17C.

The granulation tissue throughout the length of the wounds as a result of intradermal delivery is indicative of later phases of healing, compared to control and topical delivery groups with minimal granulation tissue, particularly in the central regions of the wounds (FIG. 17C). More advanced healing with a closer return to normal morphology in the intradermally treated wounds would clinically result in the future development of a smaller scar in comparison to the tissue from controls and TOP group.

In addition to H&E staining, immunohistological evaluations were performed for assessment of neovascularization in the harvested wounds. FIG. 17D depicts immunohistochemistry (IHC) staining of wound tissues against cluster of differentiation 31 (CD31), 19 days post-injury. CD31, also known as platelet and endothelial cell adhesion molecule 1 (PECAM1) is expressed on early and mature endothelial cells, platelets, and a some mononuclear blood cells and is a well-known indicator of angiogenesis^{44,45}. Superior vascularization in freshly formed skin on the wounds treated with intradermal delivery of VEGF compared to the topical administration and control groups indicate enhanced effectiveness of the method for vascularization. This observation correlates with macroscopic wound evaluation indicating the improvement of healing in chronic wounds that received VEGF intradermally.

Feasibility Assessment in a Translational Porcine Model

Following the promising outcomes of intradermal drug delivery on murine models, a similar study was conducted on a swine acute wound model with full thickness skin injury. In contrast to small animals, pig skin is anatomically and physiologically similar enough to the human skin, so it can be considered a good model to study translational feasibility of our strategy. Importantly, the wound healing mechanism in pigs is similar to that in humans and it has been shown that healing outcomes in pigs are readily translatable to humans^{46,47}. Therefore, swine models are important for evaluating the potential of wound care products for clinical translation. Full-thickness wounds were created on the dorsum of healthy Yorkshire pigs, followed by administration of VEGF either topically or intradermally. MNA was selected as the tool for intradermal delivery of VEGF in large animal studies due to its simplicity, minimal invasiveness and promising outcomes. To serve as controls, a group of wounds was treated with PBS for evaluating the potential effects of MNA administration, and a group was left untreated.

Wound healing in small animals relies on contraction, while wounds in both human and pigs are healed by a combination of re-epithelialization (regeneration) and contraction^{48,49}. Although necessary for wound healing, excessive contraction can cause hypertrophic scarring, leading to loss of original tissue structure and function^{8,12}. Furthermore, the physical properties of the contracted scar tissue can be weaker than the original tissue making it susceptible to wound reopening. As a result, reduced contraction is preferred for recovery of original tissue functionality after healing.

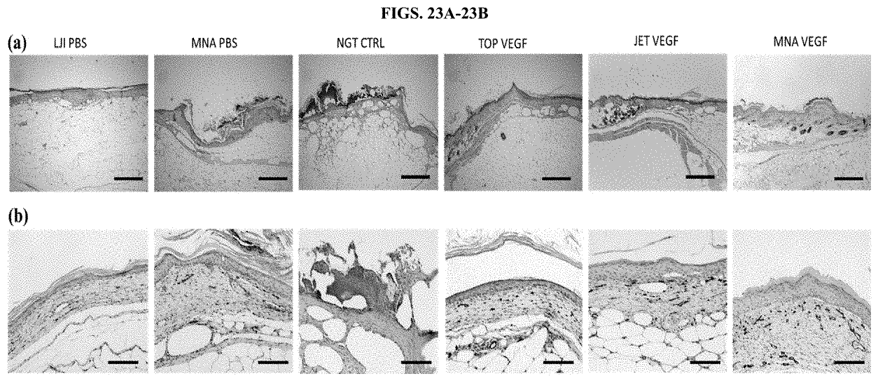

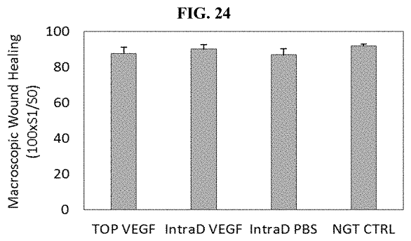

FIGS. 18A-18C demonstrates macroscopic wound healing evaluation in our large animal study. To distinguish the contribution of contraction and regeneration in wound healing, each wound's margin was marked by tattooing the animal skin at the beginning of the surgery. The shrinkage of the area surrounded by tattoos was indicative of contraction, while the wound closure without significant change in the area inside tattoos could be considered as a sign of regeneration (FIG. 18A). While the overall wound area decreased at a relatively similar rate (FIG. 24), interestingly, quantitative and qualitative analysis of the wounds in pig models demonstrated that the intradermal delivery of VEGF significantly prevented excessive wound contraction in comparison to control groups (FIGS. 18A, 18C).

Although the mechanism behind hypertrophic scarring is poorly understood, it has been suggested that dysregulated wound healing in which the apoptosis of myofibroblasts is impaired can be responsible for excessive contraction and consequently scarrine^{8,50}. Myofibroblasts with substantial expression of α-smooth muscle actin (SMA) generate contractile force to deform the wound environment while they persist extracellular remodeling and cause scarring. Therefore, excessive contraction usually coincides with hypertrophic scarring and should be reduced.

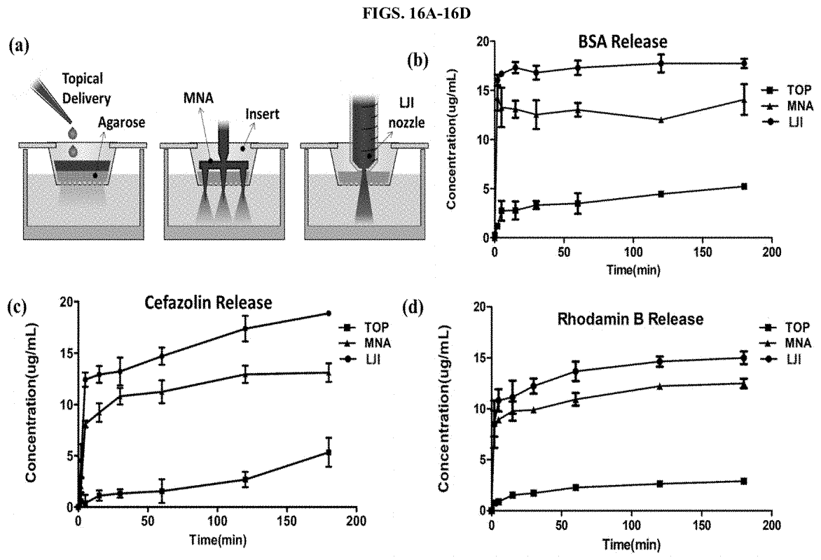

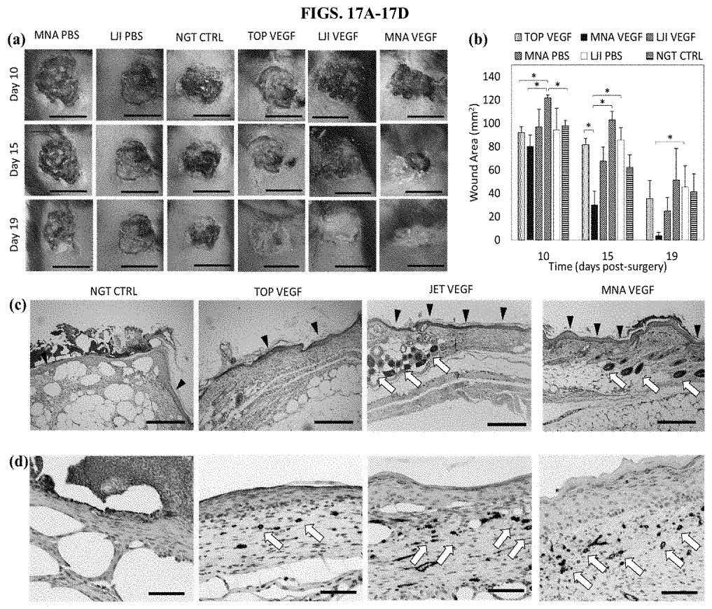

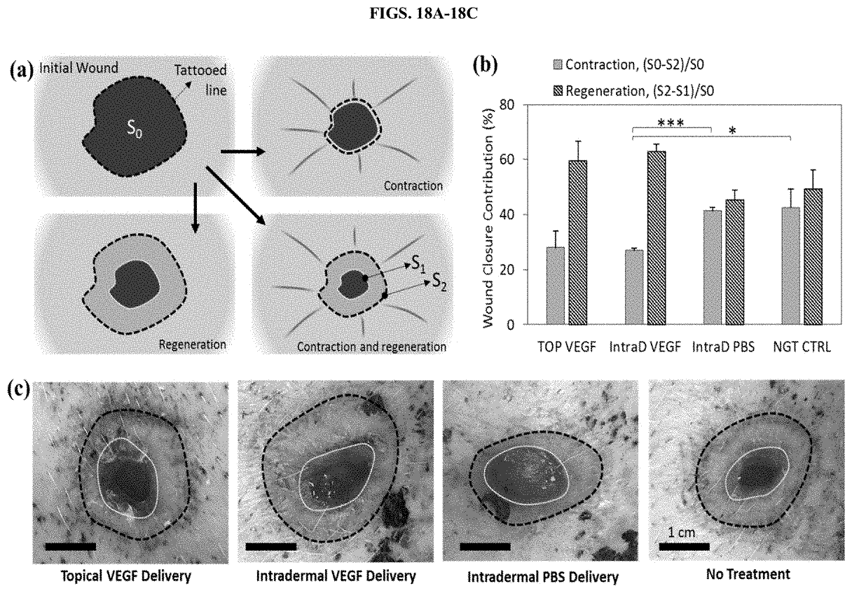

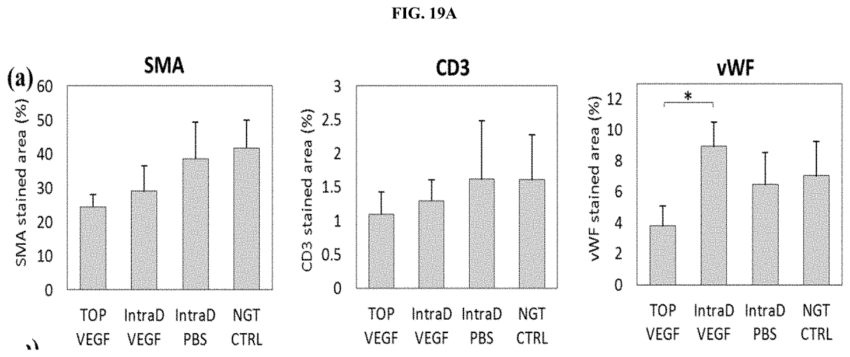

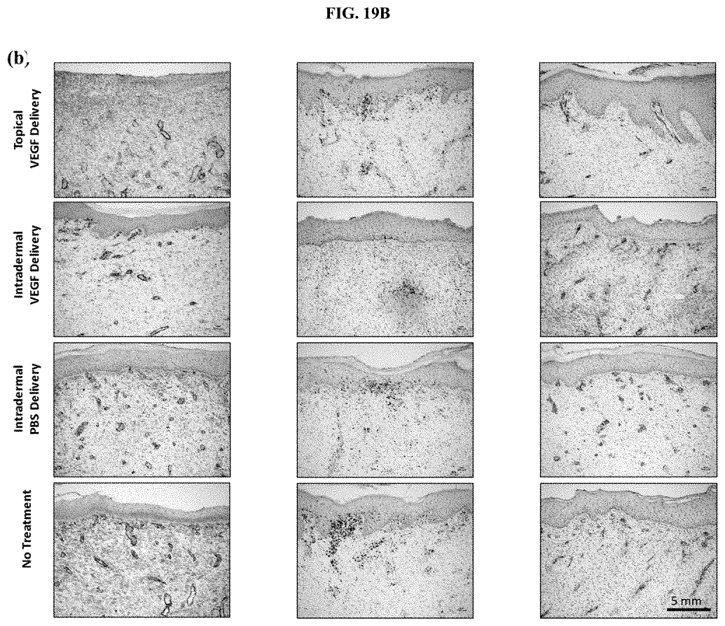

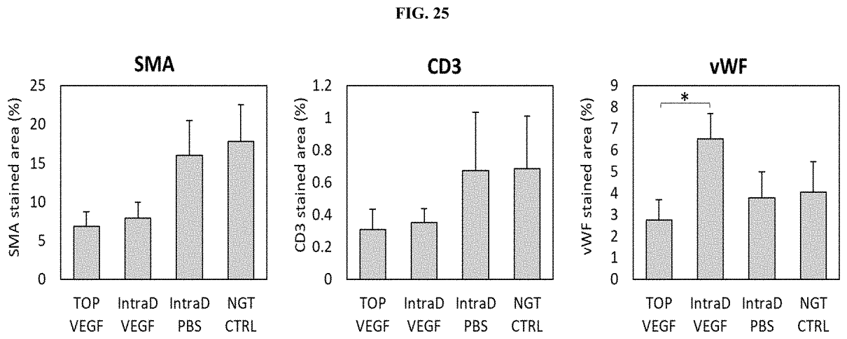

Immunohistochemical analysis further confirmed the effectiveness of our strategy for enhanced delivery of therapeutics and improved wound healing (FIGS. 19A-19C). Consistent with our macroscopic evaluations, VEGF delivery decreased the expression of SMA+ myofibroblasts responsible for contraction. Furthermore, application of the intradermal delivery tool or therapeutic reagent did not generate a significant inflammatory response (CD3 staining). More importantly, our results demonstrated a significant enhancement in vascularization throughout the depth of the granulated tissue, suggesting the effectiveness of intradermal VEGF delivery. Enhanced angiogenesis indicated by expression of von Willebrand factor (vWF) can regulate the wound environment toward a more advanced healing with minimum subsequent scarring. On the other hand, topical delivery of VEGF was clearly less effective in inducing vascularization, further highlighting the importance of the delivery tool and point in effectiveness of drugs and growth factors in wound healing. It is notable that the significant contraction in control groups can pull the native surrounding tissues characterized by proper vascularization and less presence of immune cells to the region under examination. Therefore, the effect of treatment in the groups with excessive contraction (intradermal PBS delivery and negative control) can be overestimated compared to the groups having less contraction (topical and intradermal VEGF delivery). Consequently, it is only reasonable to compare the IHC results of the groups with similar contraction. Although the comparison of the stained area between the groups with different contraction level is not proper, the group receiving VEGF via MNAs has outperformed all other groups in inducing vascularization in the wound region as evident from the IHC micrographs (FIGS. 19A-19C). A normalization of the stained areas to the non-contracted wound area, shown in FIG. 25, further demonstrates the difference on the level of inflammation and vascularization between the groups.

Discussion

A large number of patients with skin injuries suffer from either chronic non-healing wounds or over-healed scarred tissue. Chronic wounds are hard-to-treat and can cause significant morbidity. On the other hand, large acute wounds can lead to excessive contraction and scarring rather regeneration. Localized delivery of bioactive factors that can stimulate and regulate the essential biological processes has drawn significant attention. VEGF, as an angiogenic factor, can promote vascularization and eventually wound healing. In this study, the benefit of intradermal VEGF delivery for improved wound healing rate and quality was investigated.

Intradermal delivery can decrease the traveling distance of drug and eliminate eschar and exudate from its path, thereby increasing the local bioavailability of the drug delivered to the viable tissue. Intradermal delivery tools such as MNAs and LJIs can offer this advantage. To confirm this, the distributing pattern and penetration depth of topically delivered drug was compared with intradermal delivery methods using agarose gels as a tissue model. The results indicated that application of LJI and MNAs for drug delivery leads to a deeper drug penetration, and therefore can cause a more successful delivery to the wound bed compared to the topical method. The in vitro release kinetics using a wound model further confirmed this fact.

Subsequently, the benefit of intradermal VEGF delivery for healing of chronic wounds was evaluated in the diabetic murine wound model. Diabetic mice received VEGF treatment on days 5 and 7 post-injury and were monitored for their wound closure over a 19-days period. The MNAs were easy-to-use and did not harm animals, but the administration of therapeutics using LJI led to tissue injury and mortality in some animals during its use.

Although challenging to accurately quantify the wound area due to the presence of scab, the results showed an improved wound healing trend in animals receiving VEGF intradermally compared to those with topical administration of VEGF or negative controls. The histological analysis further confirmed more advanced healing stage in the wounds treated with intradermal VEGF delivery. IHC staining further supported the effect of intradermal VEGF delivery on the enhancement of vascularization, which demonstrated the effectiveness of the delivery method as well as improved healing.

Finally, clinical translation of the strategy was assessed using large animal studies. The intradermal delivery of VEGF in a swine model of full thickness skin injury reduced the wound contraction while it increased healing by regeneration. Immunohistochemical analysis further demonstrated that it enhanced the vascularization of the wound bed and reduced the level of inflammation. These findings suggest the importance of the delivery point and the administration of biological factors in the therapy outcome.

Methods

Materials: Materials including agarose, NaOH, Rhodamine B, cefazolin, and PBS were purchased from Sigma-Aldrich (MO, USA). 3D printing resin was purchased from Stratasys (VeroClear RGD 810, MN, USA). BCA Protein Assay kit (Pierce™, 23225) was obtained from Thermo Fisher Scientific (MA, USA). Reagents including recombinant Mouse VEGF 164 (Biolegend, CA, USA), anti-CD31 (primary antibody, 77699, Cell Signaling, MA, USA), ImmPRESS® HRP Horse Anti-Rabbit IgG Polymer Detection Kit (secondary antibody, MP-7401, Vector Labs, CA, USA) and DAB solution (chromogen, SK4100, Vector Labs) were used in small animal study. Recombinant pig VEGF-A (PPP030, BioRad, CA, USA), primary antibodies including von Willebrand Factor (0082, Dako, CA, USA) smooth muscle actin (ab5694, Abcam, MA, USA), and CD3 (ab16669, Abcam, MA, USA) were implemented in large animal study.

MNAs Fabrication: The computer-aided design (CAD) model of MNAs with Luer hub (which can adapt Luer lock syringes) was designed, and then an FDM 3D printer (Objet 500 Connex3, Stratasys, USA) was used to fabricate them. The needles were made of the biocompatible VeroClear resin. After fabrication, the support material was washed out by sinking samples in 3% (w/v) NaOH solution and placing it into the ultrasonic bath for a one hr. The injection system had an array of microneedles with Luer hub that could be fixed on any standard syringe (FIGS. 20A-20C).

Assessment of release kinetics: To mimic the chronic wound tissue, 12-well cell culture inserts were filled with 150 μL of 3% (w/v) agarose. Upon agarose gelation, inserts were placed in 12 well plates with wells filled with 1 mL of PBS, and then drug release kinetics was evaluated by delivering 100 μL of different solutions, namely rhodamine B solution, cefazolin and BSA, such that the final concentration of the reagents in the well would be adjusted to 20 μg/mL. Inserts were divided into 3 groups (4 replicate/group) with respect to the method of delivery, including topical, MNA, and LJI. To measure the concentration of the delivered solution, at each time point 100 μL was removed and then replaced with the same volume of fresh PBS. After collecting all samples, the concentration was measured by a plate reader (Cytation 5, BioTek) followed by data analysis to extract the related release graphs. In the case of BSA, measurement process was followed based on manufacturer's protocol.

Small Animal studies: Murine animal procedures were approved by the Institutional Animal Care and Use Committee (IACUC) of the University of Nebraska, Lincoln. Six to seven weeks old B6.BKS(D)-Lepr^{db}/J mice were obtained from the Jackson Laboratory (Bar Harbor, Me.). Since this mouse strain had congenic obesity, the animals were put on a special food diet. Mice were age and sex-matched and were divided into six groups: (1) negative control receiving no treatment (NGT CTRL, n=7); test groups receiving 100 μL of VEGF solution (500 ng/mL), either (2) pipetted on the wound topically (TOP VEGF, n=6), (3) delivered through MNAs (MNA VEGF, n=5), or (4) delivered through LJI (LJI VEGF, n=6); (5) second control group with intradermal delivery of 100 μL of PBS (LJI PBS, n=3). On day 0 of the study, 5% isoflurane was provided via a nose cone for anesthetization of animals and 3% isoflurane was used for maintaining sedation. The hair on the dorsal area was shaved, the skin was sterilized, and a full-thickness skin cut of 1 cm×1 cm was created. Buprenorphine SR was administered for pain relief at the time of surgery.

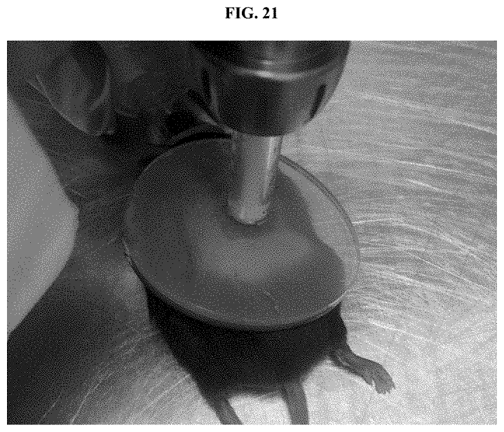

VEGF treatment started on day 5 in order to give enough time for crust formation and the creation of necrotic tissue to stimulate real wound conditions/environment. Animals received treatments on day 5 and day 7 post-surgery. A solution of 500 ng/mL of murine VEGF in PBS containing 0.1% (w/v) BSA was prepared and 100 μL of the solution was delivered topically, using MNA, or using the LJI. To minimize the potential damage of solutions delivered by LJI to mice, an approximately 9 mm thick layer of 5% (w/v) of agarose was placed between the LJI nozzle and animal skin (FIG. 21). Despite the precautions, several of the animals died due to injuries associated with high velocity liquid jet and the numbers listed above are those surviving the injections.

Animals were inspected daily for wound closure, weight change and vitals. All animals were sacrificed on day 19 post-surgery using CO₂ asphyxiation using a 30% oxygen displacement rate. The wound tissue was harvested and fixed in 10% neutral buffered formalin for further histological study.

Large animal studies: The procedures for porcine animal studies were performed at Toxikon Corporation (Bedford, Mass.). The study protocol was approved by Toxikon's Institutional Animal Care and Use Committee, and conformed to federal animal laws and regulations. Three Yorkshire pigs (Animal Biotech Industries, Danboro, Pa.), 70-80 kg each, were anesthetized by intramuscular administration of 3.3 mg/kg ketamine, 2.2 mg/kg xylazine, 1.1 mg/kg acepromazine, and 0.02 mg/kg atropine. 0-5% isoflurane and oxygen were provided to maintain general anesthesia. Pain management was achieved by administration of a transdermal patch releasing 2-3 mkg/kg/h fentanyl per hour for 72 hours (Duragesic, Janssen). In addition, buprenorphine 0.01-0.03 mg/kg was delivered intramuscularly at the end of the surgery.

Circular full-thickness wounds (d=2.5 cm) were created on the dorsum of each pig. After marking the wounds in parallel paraspinal stripes, the outlines were tattooed with red ink using an electric tattoo marker (Spaulding & Rogers Mfg., Inc., Voorheesville, N.Y.). Full-thickness wounds down to fascia were excised. Wounds were separated by at least 4 cm of unwounded skin. After wound creation, the wounds were randomly divided into 4 treatment groups (n=6 per group): (1) wounds receiving 400 μl VEGF (500 ng/mL) through MNA; (2) wounds receiving the same amount of VEGF topically; (3) control wounds treated with 400 μl of PBS through MNA; (4) second control group receiving no treatment. Subsequently all the wounds were covered with a semipermeable film dressing (Tegaderm, 3M, Saint Paul, Minn.). On day 7 post-surgery, the dressings were changed, and a second dose of similar treatment was administered. On postoperative day 14, the animals were euthanized and the wounds were photographed and harvested for histology.

For macroscopic wound evaluation, wound contraction was measured of the tattooed margins from macroscopic wound photos using Image J software (NIH, Bethesda, Md.). Open wound area (S₁) and the area inside tattooed margin (S₂) were measured and compared with original wound size on day 0 (S₀=5 cm²). The wound contraction and non-contracted wound closure contributions were calculated as 100×(S₂−S₀)/S₀ and 100×(S₂−S₁)/S₀, respectively.

Histological analysis: Sections of harvested skin were fixed by immersion in 10% neutral buffered formalin and routinely processed on a Tissue-Tek VIP 5 Tissue Processor. After processing, the tissues were embedded in paraffin blocks and were sectioned at 4 μm thick on a HM 355S Thermo Scientific microtome. Slides were deparaffinized and stained with H&E using a Leica ST5020 H&E stainer. Histological evaluation was conducted by a board certified veterinary anatomic blinded to the experiment.

Immunohistochemistry staining: In the murine study, formalin-fixed and paraffin-embedded biopsies were cut into 7 μm thick samples. Slides were deparaffinized, hydrated and washed using xylene and ethanol solutions. Subsequently, a heat mediated antigen retrieval step was performed at 95° C. for 20. The sections were blocked using 2.5% normal horse serum for 30 min at room temperature, and incubated with primary antibody (anti-CD31) at 4° C. overnight. Tissue sections were then treated with 3% hydrogen peroxide for 10 minutes at room temperature to block the peroxidase activity. Secondary antibody was then applied for 30 minutes at room temperature. To form a colored precipitate, DAB solution was incubated on the slides for 3-5 minutes. Finally, counterstaining was performed using hematoxylin for 30 seconds, and sections were dehydrated, washed, covered with mounting solution, and cover slipped.

In the swine experiment, the wounds were excised using a scalpel and fixed in formalin for immunohistochemical analysis. The biopsies were embedded in paraffin, cut in sections to give a cross section view of the wound edge-to-edge. The wound sections were immunostained for von Willebrand Factor (vWF), smooth muscle actin (SMA), and CD3 based on manufacturers' protocols and the amount of positive staining was quantified in a blinded-manner using a light microscope.

Statistical analysis: For each experiment, at least three samples were tested, and data were presented as means±standard error of mean. Comparison of the different groups was performed using a student's T test and values P<0.05 were considered statistically significant (*P<0.05, **P<0.005, ***P<0.0005). In this study, animals and their wounds were randomly assigned to MNA, LJI, TOP and control groups.

### References

1 Griffin, D. R., Weaver, W. M., Scumpia, P. O., Di Carlo, D. & Segura, T. Accelerated wound healing by injectable microporous gel scaffolds assembled from annealed building blocks. Nature Materials 14, 737-744, doi:10.1038/nmat4294 (2015).
2 Nussbaum, S. R. et al. An economic evaluation of the impact, cost, and medicare policy implications of chronic nonhealing wounds. Value in Health 21, 27-32 (2018).
3 Tamayol, A. et al. Biodegradable elastic nanofibrous platforms with integrated flexible heaters for on-demand drug delivery. Scientific Reports 7, 9220, doi:10.1038/s41598-017-04749-8 (2017).
4 Derakhshandeh, H., Kashaf, S. S., Aghabaglou, F., Ghanavati, I. O. & Tamayol, A. Smart Bandages: The Future of Wound Care. Trends in Biotechnology 36, 1259-1274, (2018).
5 Kiaee, G., Mostafalu, P., Samandari, M. & Sonkusale, S. A pH-Mediated Electronic Wound Dressing for Controlled Drug Delivery. Advanced healthcare materials 7, 1800396 (2018).
6 Broughton, G., Janis, J. E. & Attinger, C. E. The basic science of wound healing. Plastic and Reconstructive Surgery 117, 12S-34S (2006).
7 Saghazadeh, S. et al. Drug delivery systems and materials for wound healing applications. Advanced drug delivery reviews 127, 138-166 (2018).
8 Tomasek, J. J., Gabbiani, G., Hinz, B., Chaponnier, C. & Brown, R. A. Myofibroblasts and mechano-regulation of connective tissue remodelling. Nature reviews Molecular cell biology 3, 349-363 (2002).
9 Falanga, V. Wound healing and its impairment in the diabetic foot. The Lancet 366, 1736-1743 (2005).
10 Larouche, J., Sheoran, S., Maruyama, K. & Martino, M. M. Immune regulation of skin wound healing: mechanisms and novel therapeutic targets. Advances in wound care 7, 209-231 (2018).
11 Mostafalu, P. et al. Smart Bandage for Monitoring and Treatment of Chronic Wounds. Small 14, 1703509, doi:10.1002/sm11.201703509 (2018).
12 Sharpe, J. R. & Martin, Y. Strategies demonstrating efficacy in reducing wound contraction in vivo. Advances in Wound Care 2, 167-175 (2013).
13 Derakhshandeh, H. et al. A Wirelessly Controlled Smart Bandage with 3D-Printed Miniaturized Needle Arrays. Advanced Functional Materials 30, 1905544, doi:10.1002/adfm.201905544 (2020).
14 Mangoni, M. L., McDermott, A. M. & Zasloff, M. Antimicrobial peptides and wound healing: biological and therapeutic considerations. Experimental dermatology 25, 167-173 (2016).
15 Barrientos, S., Brem, H., Stojadinovic, O. & Tomic-Canic, M. Clinical application of growth factors and cytokines in wound healing. Wound Repair and Regeneration 22, 569-578 (2014).
16 Werner, S. & Grose, R. Regulation of wound healing by growth factors and cytokines. Physiological reviews 83, 835-870 (2003).
17 Chiappini, C. et al. Biodegradable silicon nanoneedles delivering nucleic acids intracellularly induce localized in vivo neovascularization. Nature Materials 14, 532-539, doi:10.1038/nmat4249 (2015).
18 Bao, P. et al. The role of vascular endothelial growth factor in wound healing. Journal of Surgical Research 153, 347-358 (2009).
19 Galiano, R. D. et al. Topical vascular endothelial growth factor accelerates diabetic wound healing through increased angiogenesis and by mobilizing and recruiting bone marrow-derived cells. The American journal of pathology 164, 1935-1947 (2004).
20 Mostafalu, P. et al. A Textile Dressing for Temporal and Dosage Controlled Drug Delivery. Advanced Functional Materials 27, 1702399, doi:10.1002/adfm.201702399 (2017).
21 Barrientos, S., Stojadinovic, O., Golinko, M. S., Brem, H. & Tomic-Canic, M. Growth factors and cytokines in wound healing. Wound repair and regeneration 16, 585-601 (2008).
22 Takahashi, H. & Shibuya, M. The vascular endothelial growth factor (VEGF)/VEGF receptor system and its role under physiological and pathological conditions. Clinical science 109, 227-241 (2005).
23 Losi, P. et al. Fibrin-based scaffold incorporating VEGF- and bFGF-loaded nanoparticles stimulates wound healing in diabetic mice. Acta biomaterialia 9, 7814-7821 (2013).
24 Yano, K., Brown, L. F. & Detmar, M. Control of hair growth and follicle size by VEGF-mediated angiogenesis. The Journal of clinical investigation 107, 409-417 (2001).
25 Simons, M. Angiogenesis, arteriogenesis, and diabetes: paradigm reassessed? Journal of the American College of Cardiology 46, 835-837 (2005).
26 Wang, P. et al. In situ formed anti-inflammatory hydrogel loading plasmid DNA encoding VEGF for burn wound healing. Acta biomaterialia 100, 191-201 (2019).
27 Leonida, M. D. & Kumar, I. in Bionanomaterials for Skin Regeneration 27-35 (Springer, 2016).
28 Enoch, S. & Harding, K. Wound bed preparation: the science behind the removal of barriers to healing. Wounds 15, 213-229 (2003).
29 Whittam, A. J. et al. Challenges and opportunities in drug delivery for wound healing. Advances in wound care 5, 79-88 (2016).
30 Kim, Y.-C., Park, J.-H. & Prausnitz, M. R. Microneedles for drug and vaccine delivery. Advanced drug delivery reviews 64, 1547-1568 (2012).
31 Larraneta, E., Lutton, R. E., Woolfson, A. D. & Donnelly, R. F. Microneedle arrays as transdermal and intradermal drug delivery systems: Materials science, manufacture and commercial development. Materials Science and Engineering: R: Reports 104, 1-32 (2016).
32 Luzuriaga, M. A., Berry, D. R., Reagan, J. C., Smaldone, R. A. & Gassensmith, J. J. Biodegradable 3D printed polymer microneedles for transdermal drug delivery. Lab on a Chip 18, 1223-1230, doi:10.1039/C8LC00098K (2018).
33 Riffle, L. Cellular mechanisms of skin repair in humans and other mammals. Journal of cell communication and signaling 10, 103-120 (2016).
34 Schramm, J. & Mitragotri, S. Transdermal Drug Delivery by Jet Injectors: Energetics of Jet Formation and Penetration. Pharmaceutical Research 19, 1673-1679, doi:10.1023/A:1020753329492 (2002).
35 Arora, A., Prausnitz, M. R. & Mitragotri, S. Micro-scale devices for transdermal drug delivery. International Journal of Pharmaceutics 364, 227-236, (2008).
36 Kunugiza, Y. et al. Acceleration of wound healing by combined gene transfer of hepatocyte growth factor and prostacyclin synthase with Shima Jet. Gene therapy 13, 1143-1152 (2006).
37 Dçbrowska, A. K. et al. Materials used to simulate physical properties of human skin. Skin Research and Technology 22, 3-14, doi:10.1111/srt.12235 (2016).
38 Zhang, D., Das, D. B. & Rielly, C. D. Microneedle assisted micro-particle delivery from gene guns: experiments using skin-mimicking agarose gel. Journal of pharmaceutical sciences 103, 613-627 (2014).
39 Schramm-Baxter, J. & Mitragotri, S. Needle-free jet injections: dependence of jet penetration and dispersion in the skin on jet power. Journal of Controlled Release 97, 527-535 (2004).
40 Mitragotri, S. Current status and future prospects of needle-free liquid jet injectors. Nature Reviews Drug Discovery 5, 543-548, doi:10.1038/nrd2076 (2006).
41 Holmes, D. I. & Zachary, I. The vascular endothelial growth factor (VEGF) family: angiogenic factors in health and disease. Genome biology 6, 209 (2005).
42 Ausprunk, D. H. & Folkman, J. Migration and proliferation of endothelial cells in preformed and newly formed blood vessels during tumor angiogenesis. Microvascular research 14, 53-65 (1977).
43 Wu, P., Fu, Y. & Cai, K. Regulation of the migration of endothelial cells by a gradient density of vascular endothelial growth factor. Colloids and Surfaces B: Biointerfaces 123, 181-190 (2014).
44 Kim, S.-W. et al. Human Peripheral Blood-Derived CD31+Cells Have Robust Angiogenic and Vasculogenic Properties and Are Effective for Treating Ischemic Vascular Disease. Journal of the American College of Cardiology 56, 593-607, (2010).
45 Schlüter, A. et al. CD31 and VEGF are prognostic biomarkers in early-stage, but not in late-stage, laryngeal squamous cell carcinoma. BMC Cancer 18, 272, doi:10.1186/s12885-018-4180-5 (2018).
46 Sullivan, T. P., Eaglstein, W. H., Davis, S. C. & Mertz, P. The pig as a model for human wound healing. Wound repair and regeneration 9, 66-76 (2001).
47 Eming, S. A., Martin, P. & Tomic-Canic, M. Wound repair and regeneration: mechanisms, signaling, and translation. Science translational medicine 6, 265sr266-265sr266 (2014).
48 Gabbiani, G. The myofibroblast in wound healing and fibrocontractive diseases. The Journal of Pathology: A Journal of the Pathological Society of Great Britain and Ireland 200, 500-503 (2003).
49 Zomer, H. D. & Trentin, A. G. Skin wound healing in humans and mice: Challenges in translational research. Journal of dermatological science 90, 3-12 (2018).
Junker, J. P., Kratz, C., Tollbäck, A. & Kratz, G. Mechanical tension stimulates the transdifferentiation of fibroblasts into myofibroblasts in human burn scars. Burns 34, 942-946 (2008).

### Example 3—Controlled-Release Transdermal Drug Delivery for Treatment of Wounds

Chronic wounds fail to proceed with normal phases of healing promptly due to their different etiology and pathophysiology. Lack of angiogenesis and microvasculature, high levels of ROS and hypoxia, longer inflammation phase, impaired cell functions, and infection cause a systemic deficiency in the healing process of chronic wounds. Each phase and layer of the tissue requires specific care to facilitate the proper healing of the wound. Hydrogels have been broadly used in drug delivery applications due to their unique structure and microenvironmental characteristics such as porosity and biocompatibility. Hydrogels present a method of delivery that is more controlled rather than burst release, which provides more beneficial outcomes in long-term wound treatment. Molecules and particles containing therapeutics, antibacterial agents and growth factors can be loaded in hydrogel porous environment and actively been released to the wound bed. The main challenge of using hydrogels in chronic wound drug delivery is the soft structure and low mechanical properties, which lack the ability to penetrate and pass through the wound scab. The placement of hydrogels within a resin-based 3D printed MNA facilitates their insertion and retraction into necrotic tissue.

The unusual pathophysiology of chronic wounds demands special care; different wound layers and levels of wounded tissue require different treatments and therapeutics depending on the healing stage. For example, keratinocyte growth factor (KGF) can be delivered to promote keratinocytes to assist in covering the wound with healthy skin while VEGF can be delivered to past through epidermis to reach the existing vasculature and encourage growth into the wound bed. The multi-length design of microneedle arrays will enable the treatment of each layer of wound with its unique required treatment depending on the phases of healing to facilitate addressing spatial challenges of chronic wound healing.

This section demonstrates development of semi-flexible multi-length 3D printed arrays of miniaturized needles for delivering drugs, growth factors, antibiotics and therapeutics to address chronic wound temporal and spatial healing requirements. Different therapeutics are encapsulated in two types of hydrogels: alginate or polyethylene glycol diacrylate (PEGDA) are loaded into multi-length MNAs. Different lengths in a single patch enable releasing various drugs at different layers of wound bed, stimulating different physiological events. Hydrogels' porous structure facilitate the loading and slow release of drug and other therapeutics. In this section, alginate and PEGDA as biocompatible hydrogels were loaded with BSA, and Rhodamine B and the release kinetic had been studied over time. VEGF delivery via gel filled MNAs was investigated in vitro with a standard wound scratch assay.

Materials and Methods

Materials and reagents including NaOH, cefazolin, (3-Aminopropyl) triethoxysilane (APTES), HUVEC culture media, BCA Protein Assay kit 23225, Human VEGF 165, FBS, Geltrex matric, and phosphate-buffered saline (PBS) were purchased from Sigma-Aldrich (MO, USA). 3D printing resins were obtained from Stratasys (MN, USA). Recombinant Mouse VEGF (VEGF 164) was purchased from Biolegend (CA, USA), Rhodamine, Micro BCA kit (ThermoFisher Scientific, Micro BCA™ Protein Assay Kit) kit, Alginate, PEGDA, Agarose

Multi-Length MNA Fabrication: One of the great challenges of microstructure fabrication is creating hollow channels. Micromolding, as one of the major types of MNAs fabrication, still deals with difficulties in forming a hollow channel inside the miniaturized needle. However, 3D printing is providing a fast method to fabricate miniaturized features with high resolution. For fabrication of miniaturized microneedles with smaller diameter, including hollow channels in between 3D printing technic was used. In this study, MNAs were fabricated by a high-resolution FDM 3D printer (Objet 500 Connex3, Stratasys, USA). First, the CAD model of MNA was designed in Solidworks, and the design was fabricated through a 3D printing process. In this type of 3D printer, parts are coated with a flexible support material while printing and, will be removed post-printing. The support material of 3D printing is a gel-like soluble material for upholding the overhangs and helps for printing small chambers and channels. In order to remove the support material, 3% (w/v) NaOH was dissolved in distilled water. For the next step, MNAs were placed in a NaOH solution inside an ultrasonic water bath (Fisher Scientific) for 2 hrs. Printing material for MNA was a resin-based polymer (Vero clear, Stratasys, USA), and biocompatibility of this material has been studied. 3D printing enables fabrication of MNAs at different needle spacing (1.5-3 mm), different needle length (0.8-3 mm), base sizes (0.5-1.5 mm), and opening diameters (0.2-0.5 mm). In this study, miniature needles with length of 3 mm and 2 mm selected to be used in different experimentations. 3D printing also is capable of printing MNAs with mixture of materials. In order to enable the MNA arrays to fit any wound type semi-flexible multi-material MNAs printed with flexible baking using a resin (Tango, Stratasys, USA) on the base. This base backing can conform to curved areas on the body, such as arms or legs. In order to evaluate mechanical strength of MNAs standard compression test and tissue test was performed on pig skin.

Gel-Loading Attachment Fabrication: The manual single needle filing process is a tedious method with a high human error chance. Moreover, working with MNAs will make it even more challenging to fill a precise amount of hydrogel solution in each of the hollow channels. In order to automate the gel loading process and avoid single needle filling errors, a precise, automated pipette-like attachment designed and fabricated using 3D printing. The multi-needle filling attachment was designed in Solidworks with conical channels to facilitate movement of fluid down and out of the reservoir. The receiving end of attachment was sized to fit a 0.2-2 mL pipette. It was 3D-printed in VeroClear on a Stratasys Objet 500 Connex3 3D printer. It is built in the exact size to fit on the end of a pipette. The pieces were cleaned using a razor blade and acupuncture needle to clear up all of the support material, with a final removal step with 5% NaOH bath overnight. The top and bottom pieces were pressed together, and the assembly was tested using 1% alginate solution. The attachment was affixed to a pipette and loaded with the solution, which was then distributed to each of 36 needles on a semi-flexible array MNAs and tested by loading with 1% alginate dyed with Rhodamine B for visualization, gel applied to semi-flexible, 36-needle array.

Release Profile Experimentation: Three different concentrations of hydrogel were studied for evaluating release kinetics of BSA as protein and Rhodamine B as neutral charge molecule from alginate and PEGDA hydrogels. BSA solution (ThermoFisher Scientific, Micro BCA™ Protein Assay Kit) mixed with three different concentrations of alginate gel (1.2%, 1.8%, and 2.5% (w/v)). This stock solution with 2000 μg/mL of BSA diluted with alginate solutions to reach 15 μg/mL final concentration of BSA. MNAs were filled with this BSA-containing alginate solution and Calcium Chloride (CaCl₂) used for crosslinking following this protocol: first placing MNAs in 3% (w/v) agarose gel containing 2% (v/v) CaCl₂ for few minutes and then pipetting additional 2% (v/v) CaCl₂ over the tops to ensure complete crosslinking.

Similar in vitro two-compartment chronic wound model explained in chapter 3 was used for all release studies and in vitro wound scratch study of gel-filled MNAs. Briefly, the crust and necrotic tissue covering chronic wounds simulated using a layer of porous agarose; 12-well cell culture inserts were used and covered with 150 mL of 3% (w/v) agarose solution (1.5% w/v). Once the agarose gel was solidified, the insert was placed in 12 well plates, the bottom chamber was filled with PBS, and the active compounds were delivered.

To perform a 3-day long release study, each needle array was placed in 0.5 mL of PBS. Samples were taken from solution at each time point over the course of 72 hrs and were stored in micro vials at room temperature over the duration of the release. For the final evaluation of protein release, 150 μL of each sample added to 96 well plates mixed with 150 μL of working reagent. Working reagent solution was made from a mixture of reagent A, B and C of BCA protein kit with the ratio of 24:24:1. Once reagents and samples were mixed, plates were incubated at 37° C. for an hour, and a plate reader (BioTech) used to read the absorption. Absorption values from the plate reader converted to concentrations using a calibration curve.

In order to study BSA release from PEGDA similar protocol was followed: base PEGDA with concentration: 23% (v/v) prepared, and BSA added to reach the concentration of 32 μg/mL. After filling the MNAs with the solution, PEGDA was photocrosslinked for approximately 30 seconds using a handheld UV light. A similar experimental protocol followed to study protein release from PEGDA over the course of 72 hours.

Results and Discussion

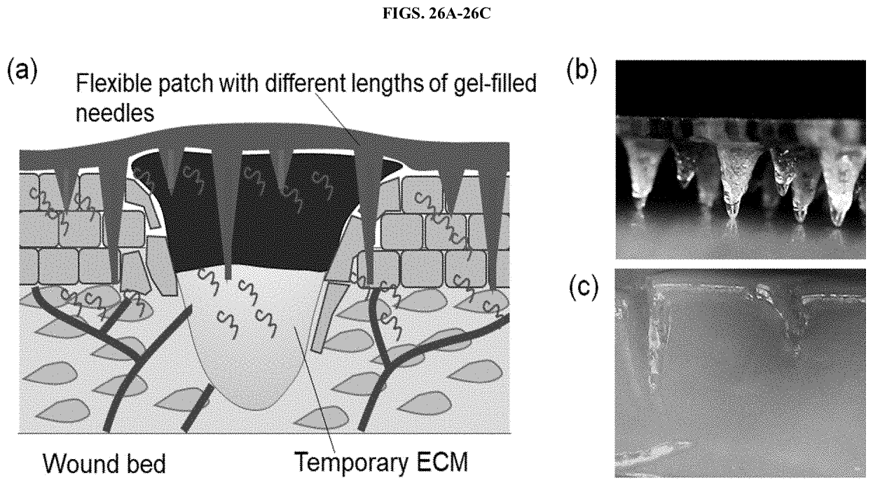

Traditional drug delivery methods fail to be effective in chronic wound healing due to their dysfunctional physiology. The existence of wound exudate and necrotic tissue lower the effectiveness and bioactivity of topically applied drugs, preventing them from getting a desirable healing result. However, each layer of wound requires specified treatment to accelerate the healing process. FIG. 26A represents schematic of a multi-level MNAs with multi-drug delivery choice to address different challenges of chronic wound healing. In this picture it's showed that longer MNAs are targeting more viable tissue by delivering VEGF to stimulating angiogenesis and vascularization, while shorter-length needles assumed to aim epidermis to deliver KGF and promote keratinocyte in upper layers of the wound. In order to examine and visualize this multi-length multi-drug concept, MNAs loaded with different color dyed gel and delivery was observed in an agarose block (FIGS. 26B and 26C).

As mentioned before, 3D printing technology enables printing of structures with different materials. FIG. 27A is a schematic illustrating steps of fabrication of 3D printed MNAs. Any part of the skin could be suffering from chronic wounds. Therefore, having a bandage with flexibility is important for conformal and full coverage on wound site. Conversely, these MNAs need to be strong enough to penetrate through necrosis and deliver therapeutics to viable tissue. To address these two challenges simultaneously the MNAs were fabricated from a rigid resin, VeroClear, capable of passing through necrosis (FIG. 27B) while the backing is printed from Tango Black to form flexible ribs to conformally cover wounds with any geometry (FIG. 27C). For precise and automated loading of the gel-precursors into the MNAs, a loading system designed, and 3D printed with a pipette attachment for filing of semi-flexible, 36-needle arrays (FIG. 27D). The demonstration of the filling process is shown in the schematic as well as gel loading and crosslinking steps. The hydrogel will be loaded into the reservoir channels' by pulling up un-crosslinked gel solution. The solution will be stored in the reservoir until it has been placed on MNA arrays to be dispensed. The solution is precisely expelled on top of each needle opening. The MNAs will be placed in a vacuum chamber for few minutes to assist degassing and complete the loading process. The MNAs will be placed in bed of agarose and CaCl₂ to be crosslinked. Needles are embedded in CaCl₂ laced agarose, so any alginate that moves down the needle is crosslinked. For completing MNAs feeling process, a vacuum chamber was used to force alginate down the needle channels. This automated system was tested by loading with 1% alginate dyed with Rhodamine-B for visualization and applied to semi-flexible, 36-needle array (FIG. 27E). Using this automated system would drastically decreases the gel-loading time for a large number of needles. FIG. 27F shows fluorescent and SEM imaging of dyed gel loaded MNAs. As shown in the picture, these MNAs provide chambers with larger capacity and surface area which can capacitate larger amount of therapeutics and facilitate better bio-interaction with chronic wound surface.

A standard mechanical test performed to study mechanical properties of 2 mm and 3 mm MNAs as well as arrays with flexible backing, semi-flexible backing, and non-flexible backing with a mechanical tester (FIG. 28A). A maximum force of 200 N was applied to both size needles, and the deformation of the MNAs was measured as a function of the applied force (FIG. 28B). As shown in FIG. 28C, MNAs did not break and were only bent under a compressive force of ˜78 N. This force is a lot greater than the force required for insertion of MNAs into a wound covered with necrosis or biofilm. A similar test was conducted to assess the penetration and retraction force of the MNAs in and out of the pig skin (FIG. 28E). No deformation or breakage was observed upon penetration and removal from the pig skin. Results indicated that the majority of the MNAs penetrated fresh pig skins with less than 2 N of force, and full penetration was achieved with about 7 N using non-flexible backing. The pull-out force for the MNAs was measured to be about 2 N. Similar results achieved in the study for insertion and extraction force using semi-flexible backing. However, the result showed that using only a flexible material (containing no rigid rein) will result no penetration or slight touch regardless of force amount. It should be noted that the overall force for the penetrating of the whole patch depends on the number of islands. The alginate adheres well to the VeroCclear, keeping it in place during insertion and removal (FIG. 28G). The best backing strategy is using semi-flexible (VeroClear needles and bases, with TangoBlack ribs). As discussed before, VeroClear is not flexible to bend around curved parts of the body, and chronic wounds are typically on the extremities because of poor blood circulation. Flexibility is a crucial property in designing a practical and ergonomic chronic wound bandage. While TangoBlack backing gives flexibility and conformality to the bandage it is not stiff enough to penetrate through the skin. Having multi-material MNA bandage, with rigid needles and flexible backing, provides enough strength to keep the needles aligned, and simultaneously allows the bandage for necessary curving and bending required to be effective on challenging body surfaces.

SEM pictures were taken for magnified demonstration and close investigation of the interface of MNAs and hydrogel in different (FIG. 29A). Furthermore, SEM image of freeze-dried gel validates the porous structure and pours distribution of alginate (FIG. 29B). This porous environment allows the drug carries and molecules to be loaded in gel structure.

Release study result of BSA and Rhodamine B from alginate with three different concentrations of gel demonstrated good release profile; curved burst release over the first 6 hours, with slower, more linear release over the next 3 days. The same trend of release was observed in experimentation of 3 mm and 2 mm MNAs. It should be considered that MNAs with 2 mm length provided smaller gel storing chamber and capacitate smaller amounts of gel compared to 2 mm MNAs. On all concentration-effect release study alginate was used as hydrogel to encapsulate BSA and Rhodamine B, the lowest concentration of the gel releases the most or equals the highest payload. Lack of strong differentiation among different concentrations and lack of constant pattern suggests that concentration might not be an incredibly impactful factor in drug release. In order to study the effect of charged hydrogel on release pattern, PEGDA was used as a base gel, and BSA release from PEGDA studied in a 3-day study. The result shows the same release pattern as alginate, demonstrating that changing the charge doesn't affect the portion release.

Conclusion

While the porous environment of hydrogels provides a suitable microenvironment for loading drugs and therapeutics and gradual and time-controlled drug release, their poor mechanical properties fail their success in chronic wound treatment due to the rigid pathology of necrotic tissue. In this study, drug-loaded hydrogels paired with 3D printed microneedles with rigid surrounding and flexible backing to provide a time-controlled drug delivery platform for chronic wound healing application. In addition, for automating the gel loading process and elimination of manual gel-filling errors, a gel-loading attachment designed and fabricated. This pipette-like attachment is capable of loading un-crosslinked hydrogel in 36-needle array in one attempt. Alginate and PEGA were used as hydrogel and BSA, and rhodamine B release has been studied. Moreover, fabrication of this platform with multi-length MNAs can equip the bandage to multi-drug delivery where each drug/growth factor can target different level of wound and address specific wound healing challenge. In order to achieve a conformal wound cover, semi-flexible multi-material bandage was 3D printed with rigid MNAs and flexible backing to well-fit wound of any geometry. The time-controlled drug release ability of gel filled MNAs was examined in vitro to evaluate effect of VEGF delivery in wound closure rate.

### Example 4—Smart Bandage for Monitoring and Treatment of Chronic Wounds

Abstract

Chronic wounds are a major health concern and they affect the lives of more than 25 million people in the United States. They are susceptible to infection and are the leading cause of non-traumatic limb amputations worldwide. The wound environment is dynamic, but their healing rate can be enhanced by administration of therapies at the right time. This approach requires real-time monitoring of the wound environment with on-demand drug delivery in a closed-loop manner. In this paper, a smart and automated flexible wound dressing with temperature and pH sensors integrated onto flexible bandages that monitor wound status in real-time to address this unmet medical need is presented. Moreover, a stimuli-responsive drug releasing system comprising of a hydrogel loaded with thermo-responsive drug carriers and an electronically controlled flexible heater is also integrated into the wound dressing to release the drugs on-demand. The dressing is equipped with a microcontroller to process the data measured by the sensors and to program the drug release protocol for individualized treatment. This flexible smart wound dressing has the potential to significantly impact the treatment of chronic wounds.

Introduction

Dermal injuries can render the human body significantly vulnerable to infections. [1] Skin possesses excellent regeneration properties that allow its rapid healing upon its injury.[2] However, some traumatic injuries that cause significant skin damage such as burns or underlying conditions such as diabetes can overwhelm the regenerative capacity of skin. [3] In such wounds, the normal healing steps of inflammation, proliferation, and maturation do not occur as expected. For example, in diabetic patients endothelial cells do not respond properly to the released cytokines and cannot support rapid angiogenesis. [4] The low oxygen content reduces the ability of immune cells to fight environmental pathogens and thus these wound ulcers become quickly non-healing. [5] Studies show that over 90% of wound ulcers that were slow to heal or recurred after clinical discharge were infected with bacteria such as Staphylococcus aureus. [6] The lack of proper immune response can lead to bacteremia and sepsis if the local infection is not treated effectively.[7] As a result, chronic wounds are one of the key causes of limb amputations.

The effective protection of the body against bacterial infection requires a combinatorial method that can: (i) rapidly cover the wound and form a barrier against environmental pathogens and (ii) prevent systemic infection by its early stage detection followed by localized or systemic treatment with antibiotics. Existing dressings cover the wound bed and in some cases release therapeutic molecules passively. However, these wound dressings provide limited information about the status of the healing process and operate in an open loop manner. Infection is a key challenge associated with chronic wounds, and patients should either be hospitalized or be continuously screened by medical professionals for signs of infection adding to treatment cost. [7,8] Smart systems that can monitor the wound environment without the need for dressing replacement and visits to medical facilities are extremely beneficial.[9] The emergence of flexible electronics has advanced the state-of-the-art of wearable smart biomedical devices for disease diagnosis and treatment.[10] Different sensors and actuators can be integrated into a single platform capable of maintaining conformal contact with skin. [10b-d,11] Despite these advancements, wound dressings are still primitive and cannot provide information about the wound status. The use of flexible sensors for measuring various parameters provides invaluable information helping with the selection of treatment strategy.[12] For example, one could monitor the pH of the wound environment, which is a strong indicator of bacterial infections. [13] The pH of normal healing wounds is in the range of 5.5-6.5 during the healing phase. However, in non-healing infected wounds, pH will be above 6.5.[13a,14] Although blood pH can also be slightly affected by diet and diseases, the variations of wound pH in response to infection are more severe. Thus, monitoring pH could provide important data on possible infection.

Moreover, advanced medical patches can deliver therapeutics in response to the wound status in a more controlled fashion than what is possible today. Current infection therapies often require oral (systemic) and topical (e.g., on the entire wound) antibiotic administration, which need much larger dosage than would be required if administered locally (e.g., only in wound regions which need it). Such routes of antibiotic administration can also have several side effects on healthy tissues or organs. A local delivery patch, however, would overcome these drawbacks to provide more precise and controlled administration of therapeutics within the wound region to promote optimal wound healing. pH-responsive materials which function through a change in their ionization and thus the polymer state have been used for engineering self-responding drug delivery tools that can automatically release their payload.[15] However, the release rate of the encapsulated antibiotics usually depends on the environment pH and might not be sufficient to remove the pathogens and create resistance. Their critical pH is also not easy-to adjust, which limit their application for treatment of different wounds. Thus, systems that benefit from a separate stimulus for drug release might be easier to use.

As a demonstration of the potential for integrated wound monitoring and treatment using emerging flexible bioelectronics, we have engineered a platform capable of in situ detection of bacterial infection by continuously monitoring wound pH, as well as administration of antibiotics locally and on demand. The electronics feature an on-board wireless transceiver that transmits sensor data and can receive instructions for programmed drug release; such wireless communication capabilities serve not only to improve healing but also to maintain remote engagement among physicians, caregivers, and patients throughout the wound care process.

Results and Discussion

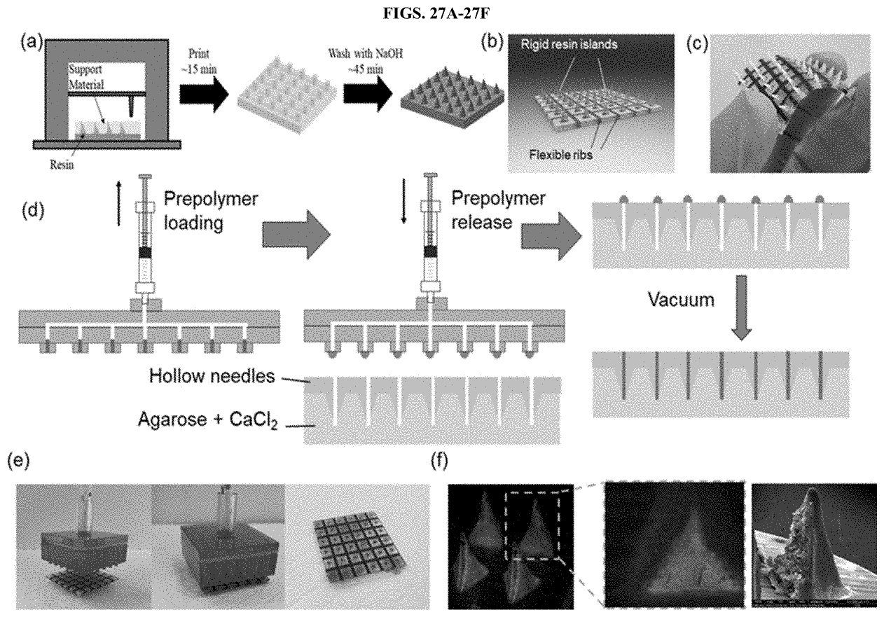

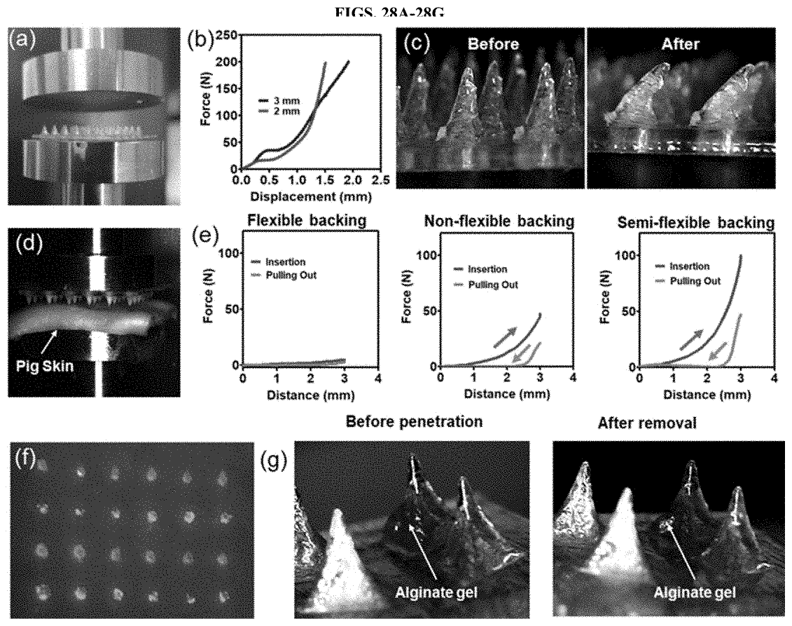

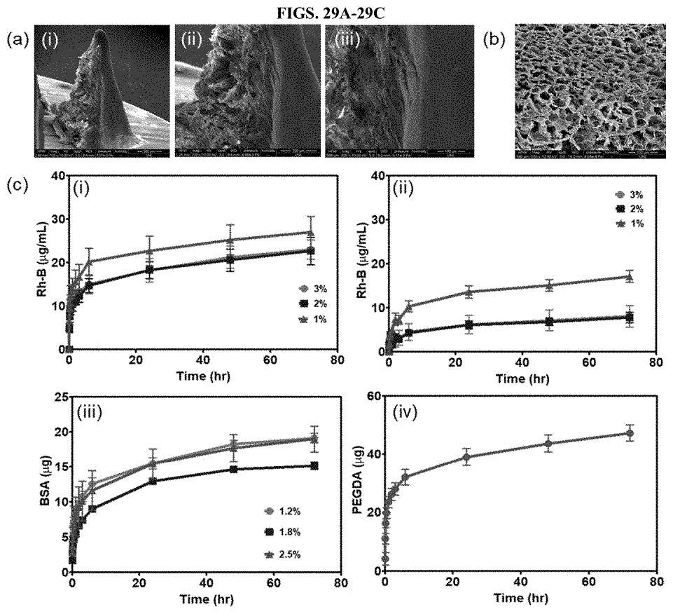

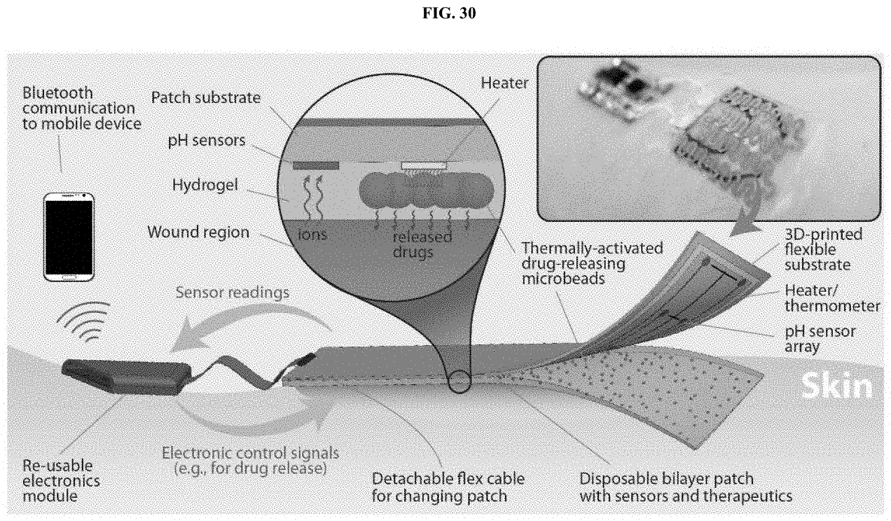

A smart bandage was engineered with multiple components including: (i) sensors (pH and temperature), (ii) microheater, (iii) thermo-responsive drug carriers embedded in a hydrogel patch, and (iv) wireless electronics to read the data from the sensors and to trigger and control the thermal actuation system if required (FIG. 30). pH is among the key parameters for monitoring of chronic wounds. pH of a chronic wound changes from acidic to alkaline, which is typically interpreted as an indication of the bacterial infection.[16] Thus, it could be used for prescreening of chronic wounds. Additionally, the temperature sensor was utilized to provide further information about the wound inflammation. Thermo-responsive drug carriers were employed for on-demand release in response to temperature variation. For this reason, PNIPAM-based particles were fabricated and embedded within an alginate hydrogel sheet. The patch was directly cast on top of a flexible heater, which in turn was controlled using the integrated microcontroller. The entire construct was attached to a transparent medical tape to form a wearable platform that was less than 3 mm thick. The platform was engineered in a way that the sensing modules and the integrated heater were low-cost and could be disposed, while the electronics could be reused.

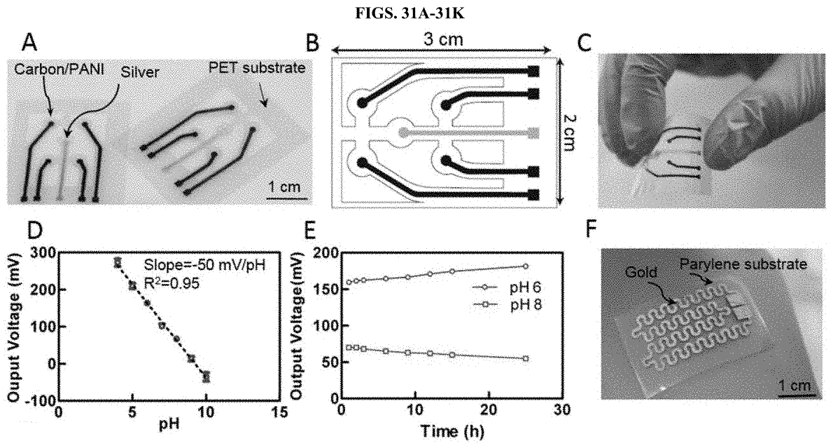

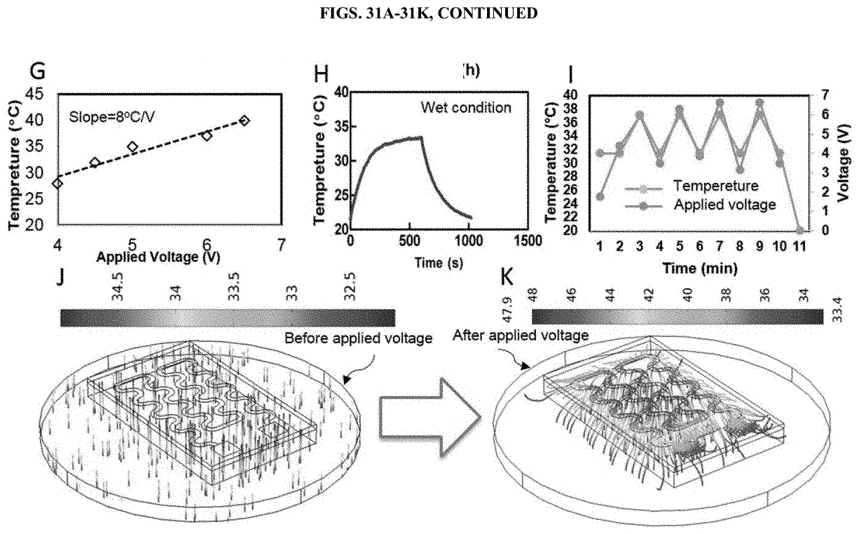

Potentiometric pH sensors were designed and fabricated as described in the Experimental Section. [17] Carbon/polyaniline (PANT) and silver/silver chloride served as working and reference electrodes, respectively, and PANI was employed as a positive exchange membrane (FIGS. 31A-31C). The principle of operation involves protonation and deprotonation of working electrode in an acid and basic environment, where charge accumulation resulted in a voltage output that could be measured for determination of pH.

The sensor function was evaluated in terms of stability and repeatability over a wide range of pH values (from 4 to 10 and back to 4). The sensors were sequentially placed in solutions containing Na+ (142 mmol) and Ca2+ (2.5 mmol) with an ionic composition similar to human exudate with different levels of pH and the generated potential between the working and reference electrodes was measured (FIG. 31D). The potential measurement of the sensor at each pH solution was recorded after reaching equilibrium state and output stability was obtained. The sensors exhibited a relatively linear response (r2=0.946) with average sensitivity of −50 mV pH-1. To evaluate stability, sensors were immersed in the highest and lowest physiological pH levels (6 and 8) and the change in the sensors potential was recorded over time. The sensor yielded a stable signal with less than 6 mV drift over a 12 h period, providing an adequate stability for wound monitoring application in which dressings are typically changed on daily basis. Currently, there is no standard test for in vitro assessment of the function of wearable sensors. Thus, to test the function of the sensor on skin, the condition was mimicked by creating agarose hydrogels (3% w/v) with different pH values. The engineered hydrogels mimic the physical properties of the wounded skin. The sensor was placed on top of them and the output signal was recorded.

A resistive microheater with resistance of 20Ω was designed and fabricated on a flexible parylene substrate, which was lightweight and FDA-approved (FIG. 31F). Electrical power was transferred to the heater using electrical driver controlled by an Arduino microcontroller (LightBlue Bean, MA). The microcontroller could also communicate with external sources wirelessly, using a low-energy Bluetooth module (LightBlue Bean, MA) assembled on the electrical board. For adjusting the temperature generated by the heater and avoiding the temperature overshoot, a commercially available flexible temperature sensor (OMEGA, CT) with a linear response and sensitivity of 10 Ω° C.-1 was assembled into the bandage next to the flexible heater. The feedback from the temperature sensor was used for adjusting the platform temperature.

The calibration graph showed that the temperature varied linearly with respect to applied electrical power (FIG. 31G). Additionally, transient graph confirmed a relatively fast response of the microheater with a time response of less than 5 min (FIG. 31H). To evaluate the feedback control of the platform for stabilizing the temperature, microheater in combination with the temperature sensor and electronic system was utilized. We showed the capability of the platform for dynamically switching between various temperatures. The platform was programmed to switch the temperature between 30 and 40° C. (FIG. 31I). Distribution of the heat over the hydrogel onto skin was predicted through numerical simulation using COMSOL Multiphysics. Simulation results before and after applied electrical power confirmed precise control on temperature distribution within the hydrogel layer (FIGS. 31J, 31K).

To be able to release antibiotics on demand, we employed stimuli-responsive drug carriers that could be triggered by applying an external stimulation. Temperature triggering mechanism was employed for delivery of antibiotics as it was safe (at temperatures lower than 42° C.) and easy-to-apply.[18] PNIPAM is a biocompatible thermo-responsive material, which can go through hydrophilic-hydrophobic transition above its critical temperature. [19] PNIPAM's critical temperature is around 32° C., which might make the drug carriers susceptible to be self-triggered. However, PNIPAM can be grafted with other monomers or copolymerized to increase its critical temperature to ≈37° C., reducing the possibility of undesired drug release.[20] PNIPAM particles were fabricated using microfluidic flow focusing approach in which the PNIPAM solution was introduced into the microchannel and was wrapped with an oil solution containing surfactant to form droplets. The size of the generated droplets could be tuned by adjusting the ratio between the flow rates of the two streams. The droplets were then crosslinked by UV irradiation. Particles with the diameter of 300 μm were fabricated, which were shrinking as being heated above 32° C. as shown in FIG. 32A. This critical temperature of the PNIPAM particles makes them suitable for topical applications where the skin temperature is less than 37° C. Details of the microparticle fabrication and drug release study can be found in the Experimental Section. These drug carriers were embedded into an alginate hydrogel layer (FIG. 32D). A thin layer of rectangular alginate patch (2% w/v) was formed by adding solution of sodium alginate counting drug carriers into PDMS molds followed by spraying calcium chloride as a crosslinker as described elsewhere. [19]

To minimize the thermal contact resistance between the microheater and the hydrogel layer, the hydrogel was directly cast on top of the heater. Chronic wounds are known for their high rate of exudate generation and thus exudate management is important in the proper function of the sensors and drug delivery modules. The utilized alginate-based hydrogels are known for high water uptake and swelling ratios and have been used in engineering wet wound dressings. In addition, since the sensors are in direct contact with the skin, the swelling of the hydrogels is not expected to affect the readout. To visualize the drug release, rhodamine B was loaded into PNIPAM particles and the system was heated while being monitored under a microscope. The rhodamine B release was observed using florescent images (see FIGS. 32E, 32F). In order to load drugs, lyophilized PNIPAM particles were soaked in cefazolin solution (10 mg mL-1) at 4° C. Cefazolin is an effective antibiotics used against S. aureus, which is the most common bacteria in infected chronic wounds. [6] After applying heat to the alginate patch, the cefazolin release profile was measured over time. Data suggested a quick burst release, which was followed by a controlled release of the drug; about 80% of the drug was released over 1 h. The release from drug carriers embedded within hydrogel was prolonged in comparison with those freely dispersed in phosphate buffered saline (PBS) (FIG. 32G). As expected, higher temperatures led to faster release rate. To assess the possibility of releasing drugs in a dynamic fashion, heat was applied periodically (30 min on, 30 min off) and the drug release amount was determined. The results suggested that after reducing the temperature, the antibiotic release rate was significantly reduced. By reapplying heat, the drug release was restored (see FIG. 32H).

A standard scratch test assay was used to evaluate the cell migration in presence of bacteria. It mimics the cell migration during healing of the infected wound and shows the efficacy of the healing. To perform this experiment, the scratch was created over a monolayer of the confluent keratinocytes, which were contaminated with S. aureus in advance. Three groups were used for this set of experiments: (1) cefazolin powder as a positive control, (2) alginate without antibiotic loading as a negative control, and (3) alginate containing cefazolin. The negative control showed the interaction of bacterial contamination with cell proliferation and migration observed through scratch closure. The positive control enabled us to compare the effect of antibiotics released from the patch with free antibiotics which indicated that the effectiveness of the antibiotics was not affected by thermal stimulation.

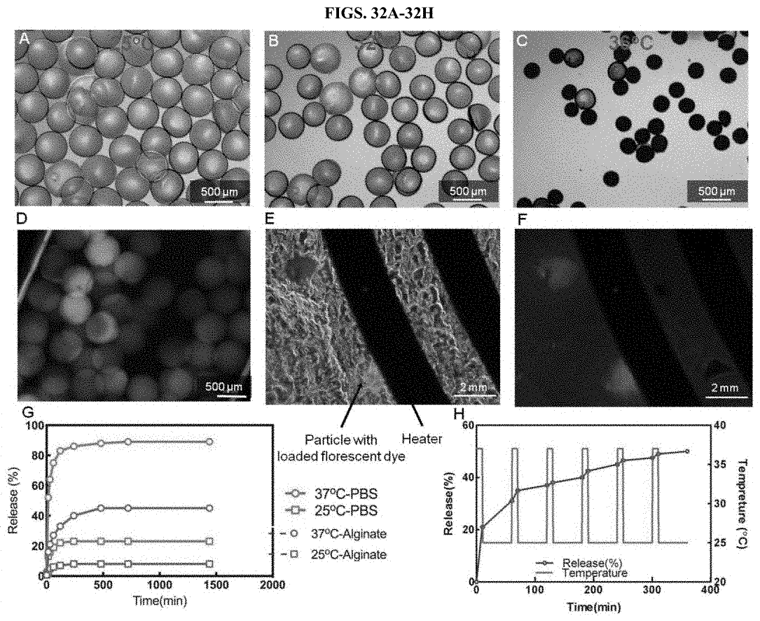

The dissolved cefazolin solution was used as a control to compare its effectiveness with the stimuli-responsive release patch made of alginate containing thermo-responsive drug carriers. Initial captured pictures showed a similar distance gap for different samples (FIGS. 33A-33F). When alginate/Ab or control samples were employed, rapid migration toward the opening area was observed. Moreover, the whole distance gap was covered with migrated cells eventually for these samples. On the other hand, the efficient migration was not seen when alginate patch without antibiotics was used.

Wound dressing should not be cytotoxic or should not negatively affect cellular growth. Although animal studies are the most suitable tool for preclinical assessment of the function of the wound care products, in vitro culture of human cells is also strong tool for preliminary studies. Thus, the engineered dressings were interfaced with the culture of human keratinocytes and potential toxicity of the engineered dressing was assessed. As it is shown in FIGS. 33E, 33F, the cell viability and total DNA content in presence of the antibiotic-releasing patch were similar to the control patch with viability of more than 90%.

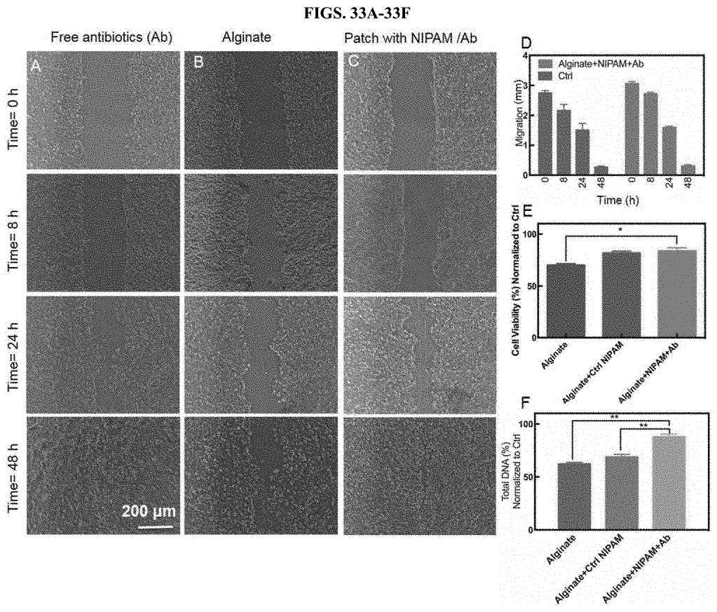

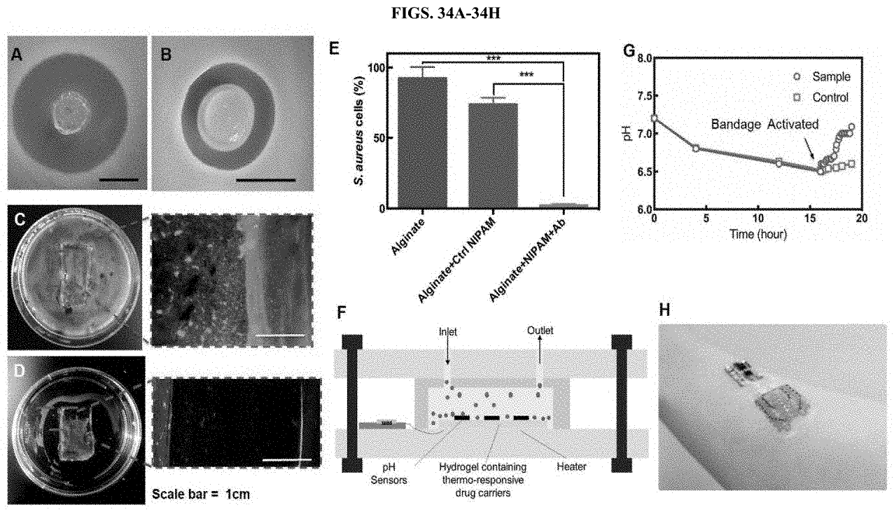

To evaluate the efficacy of the thermo-responsive delivery of the antibiotics, zone of inhibition (ZOI) and number of colony-forming units (CFU) tests were performed. For the ZOI test, S. aureus was cultured and added on the surface of an agar plate. Then, two sets of the hydrogel patches with and without antibiotics were placed on the bacterial culture. To prepare the hydrogel patch, PNIPAM particles were loaded with cefazolin and embedded inside the alginate hydrogel. The heater was triggered to release the drug. Significant ZOI mm) around the patch loaded with antibiotics was observed in comparison to the control confirming the effectiveness of the released drug in inhibiting bacterial growth (FIGS. 34A-34B).

CFU experiment was also performed to further quantify the efficacy of the thermo-responsive antibiotic-releasing patch. While the alginate patch without antibiotics as a negative control showed ≈100% viability ratio of the bacteria, the use of the antibiotic-loaded patch reduced their viability ratio to less than 10% (FIG. 34E). Moreover, the capability of the antibioticeluting patch on the treatment of bacterial infection was tested on a biofilm layer of the bacteria. Bacteria were cultured on the agar plates for 4 d to create a biofilm. Then, the patch with integrated microheater was placed on the top. Live-dead assay was used after 6 h to assess the viability of bacteria at the interface of the patch. We observed that bacteria had considerable spread over the patch and the plate in the absence of the antibiotic (FIG. 34C), while bacteria were killed significantly at the interface of the antibiotic eluting patch (FIG. 34D).

In another experiment, a bioreactor was designed to evaluate the pH variation over the bacterial culture before and after activation of the smart patch (FIG. 34F). Bacteria culture media was perfused slowly to mimic the in vivo condition. As shown in FIG. 5G, when bacteria reached the lag phase, the pH was reduced to 6.5 (the critical pH defined for the microcontroller in this experiment). At this pH, the bandage was automatically activated and cefazolin was released. pH raised up to 7.2 in the bioreactor with activated bandage whereas a stable pH was seen in the nontreated sample (deactivated bandage).

The entire platform was packaged into a flexible and wearable form that could form conformal contact with skin. A transparent medical tape was used for securing it onto skin. A typical fabricated bandage with the utilized electrical system is shown in FIG. 34H.

Conclusions

In this work, a networked closed-loop automated patch for monitoring and treatment of the chronic wounds was developed. The closed-loop patch included sensor patch of flexible pH and temperature sensors, a hydrogel release patch with thermos-responsive drug-loaded carriers with integrated microheater, and an electronics patch. Flexible electrochemical pH sensor with linear response and sensitivity of −50 mV pH-1 was designed and fabricated on flexible PET substrate. According to the feedback data from pH sensor, thermo-responsive release patch was activated for the release of the antibacterial drugs.

Readout from the sensor and stimulation of thermo-responsive drug were achieved remotely by taking advantage of the wireless Bluetooth low energy module in the electronics patch. The proposed bandage was characterized by in vitro bacterial study and subsequent antibiotic release. Additionally, scratch test assay was performed to prove the cell migration over scratch. In the proposed smart bandage, pH and temperature sensors and antibiotic drug serve as models of sensors and the drug, one could embed more sensing components, drugs, and growth factors in this platform for specific detection of particular healing marker and for treatment of different target conditions. The assessment of clinical advantage of the smart and automated (feedback controlled) dressing for facilitating the healing process of chronic wounds and its comparison to other existing technologies and wound care products requires the use of animal models that represent difficult-to-treat wounds which will be carried out in the future.

Experimental Methods

Materials: Chemicals including sodium alginate, agarose, CaCl₂, PNIPAM, N,N′-methylenebisacrylamide, and mineral oil were purchased from Sigma-Aldrich (St. Louis, Mo.). Irgacure 2959 (CIBA Chemicals) was used as the photoinitiator (PI). Cell culture reagents were obtained from Life Technologies, MA.

Fabrication and Characterization of pH Sensor: The pH sensors were prepared based on a previous work. [17] Briefly, the pH sensor consisted of a working electrode and a solid-state Ag/AgCl reference electrode. The voltage read across the two electrodes corresponded to the pH level of the analyte solution. The fabrication process was conducted by laser-machining and screen-printing. The fabrication process starts by laser cutting a single layer of tape (3M MagicTape) to act as a stencil mask for the screen-printing process. The mask was then attached to the flexible PET substrate followed by screen-printing the carbon (MG Chemicals Graphite Conductive Coating) and silver inks (118-09, Creative Materials, Ayer, Mass.). After the printing process the mask was removed, revealing four working electrodes and one reference electrode. The inks were then cured in an oven set to 80° C. for 1 h.

Using the same laser machine and settings, the perimeter of electrodes was removed from the PET substrate. The openings created in the PET substrate allow the interface between the drug delivering hydrogel and the wound. Next, to prevent electrical crosstalk between the electrodes an insulating layer of poly(methyl methacrylate) (PMMA; A4) was printed onto the conductive carbon and silver traces, which defines the active area and the contact pad of the sensors. The insulating layer was cured by hot plate set to 80° C. for 10 min followed by exposing to UV light for 5 min.

The Ag/AgCl reference electrode was prepared by electrodepositing a layer of AgCl over the silver electrode. The chloridization process was performed in a solution of 1.0 m NaCl and a constant current of 1 mA for 3 min was applied across the silver electrode and a Pt electrode. The uncovered area of silver electrode changes in color with the formation of silver chloride. The resulting Ag/AgCl electrode was then rinsed with deionized water and blow dried with nitrogen.

The pH-sensitive membrane was prepared by dissolving 25 mg of polyaniline emeraldine in 10 mL dimethyl sulfoxide (DMSO), both purchased from Sigma-Aldrich, MA. For a complete dissolution, the mixture was placed in an ultrasonic bath for 2 h followed by stirring with magnetic rod for 24 h. The solution was then remained undisturbed for 24 h to allow the undissolved particles to settle. The solution was decanted and filtered through a 500 nm filter (Whatman filters) yielding a uniform dark blue solution. The working electrode was prepared by drop-casting 5 μL volume of the pH polyaniline emeraldine base solution onto the active area of the carbon electrodes and slowly dried at 50° C. for 2 h. The polyaniline film was then doped with H+ ion by placing the device in a vacuum chamber with 2 mL of 1 m HCl for 5 h. The HCl fumes induced by low pressure in the vacuum chamber introduces H+ ions into the polyaniline emeraldine based membrane, which was then converted to polyaniline emeraldine salt. The electrodes were then rinsed with DI and dried with nitrogen.

The solid-state reference membrane was made by mixing 1:1 ratio of fine KCl powdered (Sigma-Aldrich) with UV-curable adhesive (Henkel Loctite 3105). The blend was thoroughly mixed using ultrasound, forming a uniform slurry. The slurry was then cast onto the Ag/AgCl electrode and cured under UV light for 10 min. During the UV exposure, the working electrode was protected with an aluminum foil cover. The measurements were conducted in different pH buffer solutions ranging from pH of 4 to pH of 10. The buffered solutions were purchased from Nova Analytics (Pinnacle pH Buffers). The performance of the fabricated sensors was assessed by potentiometric measurements across the pH-sensitive working electrode Ag/AgCl reference electrode with using a BASI potentiostat.

Fabrication of Microheater and Temperature Sensor: Microheater was designed and prepared based on a previous work. [21] For fabrication of the microheater, first a glass wafer was covered by 25 μm parylene as a substrate using parylene coater (PDS2010) with 20 g of dimer. The wavy geometry of the microheater was designed in CAD software to be used as a mask. Adhesive mask was prepared with a laser cutter (Versa, VLS2.40) (power 40%, speed 20%) and attached to the substrate. A 20 nm chromium as an adhesive layer and a 200 nm gold layer were sputtered subsequently. Then, adhesive mask was peeled off and left behind a wavy pattern with 20Ω resistance.

For the characterization of the microheater using electronic driver, constant voltage was applied using an electronic driver board.

Particle Characterization and Drug Loading: A microfluidic coflowing device was used to fabricate PNIPAM microparticles. In this system, a 10% (w/v) PNIPAM, 0.3% (w/v) N,N-methylene-bis-acrylamide (BIS), and 0.5% (w/v) of PI solution in water was injected through the central nozzle using a syringe pump. A solution of mineral oil containing 20% (v/v) of Span80 as surfactant was injected using another syringe pump to form a sheath around the central stream. The core flow was broken into spherical droplets and the droplet size could be tuned by changing the flow rates using two syringe pumps. Once the microdroplets have been formed, they are collected in a Petri dish and are polymerized by UV-light for 3 min in the temperature around 4° C. and the UV intensity of 850 mW and the distance between the tip of the fiber optic and the Petri dish is set to 6 cm. Subsequently, the microhydrogels were washed with ethanol and distilled water several times to remove the environmental oil and then were verified under microscope visually, after that the particles were freeze-dried and stored in 4° C. for future experiments.

The freeze-dried microcarriers were immersed in distilled water with drug (concentration) and they swell promptly to their original size while absorbing the drug. When temperature reached above lower critical solution temperature (LCST) phase transition occurred and the particles change from hydrophilic polymer to a hydrophobic polymer, this alteration led to an ejection of water. This drying and swelling behavior was a reversible process that did not cause morphological damage.

Drug Release with Integrated Heaters: Alginate hydrogel with embedded drug microcarriers was attached on the surface of the microheater while 200 μL PBS was added into the plate and electrical voltage was applied to generate heat. The drug released into the solution was evaluated using plate reader.

Bacterial Study: A single colony of S. aureus was inoculated into 10 mL liquid broth (LB) culture medium overnight. For preparation of the fresh bacteria solution, 100 μL of this sample were added to a 10 mL LB and incubated at 36° C., 200 rpm overnight. After being incubated and reaching OD₆₀₀ of ≈0.4, 1 mL of the fresh bacteria was spread on an LB agar plate, the strain enters the exponential period of growth and the culture broth was diluted. Bacteria with the concentration of ≈1×10⁵ cells mL⁻¹ were cultured for ZOI and CFU counting experiments. To investigate the application of device on bacteria, the device was placed on the surface of cultured bacteria, and different electrical voltages were applied to the conductive pattern to generate heat. A blank control sample without antibiotics was prepared for comparison. All the plates were incubated at 37° C. for an appropriate time. Finally, the plates were taken out of the incubator and the ZOI and number of remaining CFUs were calculated.

Scratch Wound Healing Assay and Cell Studies: Adult Normal Human Epidermal Keratinocytes purchased from ATCC were cultured in a DMEM-based medium containing 10% FBS and 1% penicillin/streptomycin. For cytotoxicity assessment, circular samples (1 cm in diameter) were prepared and placed at the bottom of 24-well polystyrene plates. Then, cells were resuspended in culture media at the concentration of 1×10⁷ cells mL⁻¹ and 5000 cells were seeded on the samples. The samples were incubated for 1 h to allow the cells to attach and then 300 μL of culture medium was added to each well. Cellular metabolic activity was measured using PrestoBlue assay on days 1, 3, and 7 as per manufacturer's protocol. The fluorescent intensity of the assay was measured using a BioTek UV/vis Synergy 2 microplate reader.

Keratinocytes (1×10⁵) were seeded and cultured in a six-well plate and kept overnight to make a confluent monolayer overnight. The 1 mm wide gap scratch was then created onto the monolayer with a 200 μL pipette tip, and floating cells were removed by twice washing with PBS. After the line scratches, 2 mL of DMEM was added for each group. To detect the efficacy of treatments on cell migration, samples were continually imaged for 0, 8, 24, and 48 h. Images of the monolayer were taken by a microscope and cell migration activity was evaluated as the migration distance from the edges of the scratch toward the center of it using Image J software (NIH, Bethesda, Md., USA). For each well, ten images were taken and selected randomly. Experiments were done independently in triplicate.

### References

[1] Y. Bayram, M. Parlak, C. Aypak, I. Bayram, Int. J. Med. Sci. 2013, 10, 19.
[2] A. J. Singer, R. A. Clark, N. Engl. J. Med. 1999, 341, 738.
[3] T. Bjarnsholt, K. Kirketerp-Møller, P. Ø. Jensen, K. G. Madsen, R. Phipps, K. Krogfelt, N. Høiby, M. Givskov, Wound Repair Regener. 2008, 16, 2.
[4] a) H. A. Hadi, J. Al Suwaidi, Vasc. Health Risk Manage. 2007, 3, 853; b) M. G. Tonnesen, X. Feng, R. A. Clark, J. Invest. Dermatol. Symp. Proc. 2000, 5, 40.
[5] G. S. Schultz, R. G. Sibbald, V. Falanga, E. A. Ayello, C. Dowsett, K. Harding, M. Romanelli, M. C. Stacey, L. Teot, W. Vanscheidt, Wound Repair Regener. 2003, 11, 51.
[6] J. S. Boateng, K. H. Matthews, H. N. Stevens, G. M. Eccleston, J. Pharm. Sci. 2008, 97, 2892.
[7] G. A. James, E. Swogger, R. Wolcott, P. Secor, J. Sestrich, J. W. Costerton, P. S. Stewart, Wound Repair Regener. 2008, 16, 37.
[8] R. Edwards, K. G. Harding, Curr. Opin. Infect. Dis. 2004, 17, 91.
[9] S. Saghazadeh, C. Rinoldi, M. Schot, S. S. Kashaf, F. Sharifi, E. Jalilian, K. Nuutila, G. Giatsidis, P. Mostafalu, H. Derakhshandeh, K. Yue, W. S. A. Memic, A. Tamayol, A. khademhosseini, Adv. Drug Delivery Rev. 2018, 127, 138.
[10] a) P. Mostafalu, S. Sonkusale, Biosens. Bioelectron. 2014, 54, 292; b) S. Bauer, Nat. Mater. 2013, 12, 871; c) D.-H. Kim, N. Lu, R. Ma, Y.-S. Kim, R.-H. Kim, S. Wang, J. Wu, S. M. Won, H. Tao, A. Islam, Science 2011, 333, 838; d) J. A. Rogers, T. Someya, Y. Huang, Science 2010, 327, 1603.
[11] a) A. H. Najafabadi, A. Tamayol, N. Annabi, M. Ochoa, P. Mostafalu, M. Akbari, M. Nikkhah, R. Rahimi, M. R. Dokmeci, S. Sonkusale, Adv. Mater. 2014, 26, 5823; b) W. H. Yeo, Y. S. Kim, J. Lee, A. Ameen, L. Shi, M. Li, S. Wang, R. Ma, S. H. Jin, Z. Kang, Adv. Mater. 2013, 25, 2773.
[12] a) N. Lu, C. Lu, S. Yang, J. Rogers, Adv. Funct. Mater. 2012, 22, 4044; b) L. Gao, Y. Zhang, V. Malyarchuk, L. Jia, K.-I. Jang, R. C. Webb, H. Fu, Y. Shi, G. Zhou, L. Shi, Nat. Commun. 2014, 5, 4938.
[13] a) L. A. Schneider, A. Korber, S. Grabbe, J. Dissemond, Arch. Dermatol. Res. 2007, 298, 413; b) M. C. Varghese, A. K. Balin, D. M. Carter, D. Caldwell, Arch. Dermatol. 1986, 122, 52; c) R. F. Diegelmann, M. C. Evans, Front. Biosci. 2004, 9, 283; d) A. Tamayol, M. Akbari, Y. Zilberman, M. Comotto, E. Lesha, L. Serex, S. Bagherifard, Y. Chen, G. Fu, S. K. Ameri, W. Ruan, E. L. Miller, M. R. Dokmeci, S. Sonkusale, A. Khademhosseini, Adv. Healthcare Mater. 2016, 5, 711.
[14] R. Rahimi, M. Ochoa, A. Tamayol, S. Khalili, A. Khademhosseini, B. Ziaie, ACS Appl. Mater. Interfaces 2017, 9, 9015.
[15] N. Ninan, A. Forget, V. P. Shastri, N. H. Voelcker, A. Blencowe, ACS Appl. Mater. Interfaces 2016, 8, 28511.
[16] D. M. Ansell, J. E. Kloepper, H. A. Thomason, R. Paus, M. J. Hardman, J. Invest. Dermatol. 2011, 131, 518.
[17] R. Rahimi, M. Ochoa, T. Parupudi, X. Zhao, I. K. Yazdi, M. R. Dokmeci, A. Tamayol, A. Khademhosseini, B. Ziaie, Sens. Actuators, B 2016, 229, 609.
[18] A. Tamayol, A. H. Najafabadi, P. Mostafalu, A. K. Yetisen, M. Commotto, M. Aldhahri, M. S. Abdel-wahab, Z. I. Najafabadi, S. Latifi, M. Akbari, N. Annabi, S. H. Yun, A. Memic, M. R. Dokmeci, A. Khademhosseini, Sci. Rep. 2017, 7, 9220.
[19] S. Bagherifard, A. Tamayol, P. Mostafalu, M. Akbari, M. Comotto, N. Annabi, M. Ghaderi, S. Sonkusale, M. R. Dokmeci, A. Khademhosseini, Adv. Healthcare Mater. 2016, 5, 175.
[20] P. Mostafalu, G. Kiaee, G. Giatsidis, A. Khalilpour, M. Nabavinia, M. R. Dokmeci, S. Sonkusale, D. P. Orgill, A. Tamayol, A. Khademhosseini, Adv. Funct. Mater. 2017, 27, 1702399.
[21] P. Mostafalu, S. Amugothu, A. Tamayol, S. Bagherifard, M. Akbari, M. R. Dokmeci, A. Khademhosseini, S. Sonkusale, presented at IEEE Biomedical Circuits and Systems Conf. (BioCAS), Atlanta, Ga., USA, October 2015.

1. A microneedle device comprising an array of microneedles on a substrate, a reservoir configured to hold a therapeutic agent, and an applicator that, when placed at a wound site, administers the therapeutic agent from the reservoir to the wound site via the microneedles.

2. The microneedle device of claim 1, wherein each microneedle of the array has a needle length of about 0.8 mm to about 3 mm and a base size of about 0.5 mm to about 1.5 mm.

3. The microneedle device of claim 1, wherein the microneedles are hollow and have an opening diameter of about 0.2 mm to about 0.5 mm.

4. The microneedle device of claim 1, wherein the microneedles have a cone shape, a pyramid shape, a cylinder shape, a prism shape, or a pencil-like shape.

5. The microneedle device of claim 1, wherein the distal end of each hollow microneedle comprises a hole.

6. The microneedle device of claim 1, wherein each microneedle of the array is a solid microneedle.

7. The microneedle device according to claim 1, wherein the substrate is characterized by its flexibility such that when the substrate contacts an object it substantially conforms to the object's surface.

8. The microneedle device according to claim 1, wherein the applicator is a micropump, a manual pump, or a syringe in fluid communication with the reservoir.

9. The microneedle device according to claim 1, wherein the device further comprises a control module configured to communicate with the applicator and an external source.

10. The microneedle device of claim 9, wherein the control module wirelessly communicates with the external source.

11. The microneedle device of claim 10, wherein the external source is a low-energy Bluetooth module.

12. The microneedle device according to claim 1, wherein the microneedles comprise a thermoplastic resin.

13. The microneedle device according to claim 1, wherein the microneedles comprise a biodegradable resin.

14. The microneedle device according to claim 1, wherein the microneedles are produced by three-dimensional printing.

15. The microneedle device of according to claim 1, wherein the device further comprises a potentiometric pH sensor.

16. A method of treating a wound comprising:
(a) applying a wound care product to the wound, the wound care product comprising an array of microneedles on a flexible substrate, a reservoir configured to hold a therapeutic agent, and an applicator for administering the therapeutic agent from the reservoir to a wound via the microneedles; and
(b) applying pressure to the wound care product such that hollow microneedles of the array penetrate the wound, thereby transdermally or intradermally administering the therapeutic agent to the wound via the penetrating microneedles and treating the wound.

17. The method of claim 16, wherein the wound care product is a programmable wound dressing comprising a control module coupled to and in communication with the microneedle array and the applicator, and configured for controlling transdermal or intradermal delivery of the therapeutic agent from the reservoir to the wound via the microneedle array.

18. The method of claim 16, wherein at least one microneedle of the array is hollow and has a needle length of about 0.8 mm to about 3 mm, a base size of about 0.5 mm to about 1.5 mm, and an opening diameter of about 0.2 mm to about 0.5 mm.

19. The method of claim 16, wherein the microneedle array comprises one or more solid microneedles.

20. The method of claim 16, wherein said wound is a chronic wound.

21. The method of claim 20, the chronic wound comprising a bacterial infection or biofilm.

22. The method of claim 16, wherein the one or more therapeutic agents are selected from a growth factor, an antibacterial, an anti-infection agent, anti-inflammatory agent, and an anti-biofilm agent, or a combination thereof.

23. The method of claim 19, wherein the growth factor is vascular endothelial growth factor (VEGF).

24-39. (canceled)
